SEQUENCE LISTING (1 sequence)

SEQ ID NO: 1 - DNA, 21 bp, Homo sapiens
aagctaagcctcggttacaga

(19) United States
(12) Patent Application Publication
(10) Pub. No.: US 2014/0322704 A1
(43) Pub. Date: Oct. 30, 2014

(54) Hepatitis Virus Culture Systems Using Stem Cell-Derived Human Hepatocyte-Like Cells and Their Methods of Use

(71) Applicant: The Florida State University Research Foundation, Inc., Tallahassee, FL (US)

(72) Inventors: Hengli Tang, Tallahassee, FL (US); Xianfang Wu, Tallahassee, FL (US)

(21) Appl. No.: 14/366,467

(22) PCT Filed: Dec. 19, 2012
(86) PCT No.: PCT/US12/70636

§ 371 Date: Jun. 18, 2014

Related U.S. Application Data
(60) Provisional application No. 61/577,421, filed on Dec. 19, 2011.

(51) Int. Cl. C12N 7/00; C12Q 1/02; C12N 5/071

(52) U.S. Cl. CPC C12N 7/00; C12N 5/067; C12Q 1/025
USPC 435/5; 435/370; 435/235.1

(57) ABSTRACT

The invention relates to the discovery that DHH derived from stem cells are permissive for infection by hepatitis viruses (HV), such as hepatitis A virus (HAV), hepatitis B virus (HBV), hepatitis C virus (HCV), hepatitis D virus (HDV) and hepatitis E virus (HEV). Included in the invention are HV-permissive DHHs and methods of making an HCV-permissive DHH derived from a stem cell. Also included is an HV culture system comprising at least one HV-permissive DHH. The HV-permissive DHH and HV culture system are useful for conducting HV life cycle analyses, diagnosing a subject as being infected with HV, genotyping and characterizing the HV of a subject infected with HV, detecting drug resistance of HV obtained from a subject infected with HV, screening for and identify modulators of HV infection, and monitoring the effect of a treatment of HV in a subject.

### CROSS-REFERENCE TO RELATED APPLICATIONS

This application claims priority to U.S. Provisional Patent Application No. 61/577,421, filed on Dec. 19, 2011, which is hereby incorporated herein by reference in its entirety.

### BACKGROUND OF THE INVENTION

Chronic infection by hepatitis viruses, including hepatitis B virus (HBV) and hepatitis C virus (HCV), inflicts more than 550 million people worldwide and causes serious liver diseases such as cirrhosis and hepatocellular carcinoma (HCC) (2003, Alter, Semin Liver Dis. 23:39-46; 2005, Shepard et al., Lancet Infect Dis. 5:558-567). These end-stage diseases destroy the self-generating ability of the organ and require liver transplantation for patient survival. Unfortunately, in addition to the issue of donor shortage, HCV-related liver transplant patients, who account for almost half of those on the waiting list, are confronted by the serious problem of re-infection of the new graft. The current re-infection rate is 100% and disease progression appears to accelerate post re-infection (2005, Brown, Nature. 436:973-978). An alternative to solid liver organ transplant is hepatocyte transplantation, which should alleviate the shortage of donor organs (2010, Dhawan et al., Nat Rev Gastroenterol Hepatol. 7:288-298) and may alter disease progression if the hepatocytes could be modified prior to engraftment. Studies with immunodeficient mouse models indeed demonstrated that purified primary human hepatocytes (PHHs) can repopulate damaged mouse liver after transplantation (2001, Mercer et al., Nat Med. 7:927-933; 2010, Bissig et al., J Clin Invest. 120:924-930; 2011, Washburn et al., Gastroenterology 140:1334-1344). Obtaining sufficient amounts of genetically modified PHHs, however, hasn't been possible as these cells are highly limited in their proliferative ability ex vivo, precluding their expansion and restricting genetic modification; in addition, uninfected PHHs will necessarily be from a different individual than the recipient, presenting the risk of transplant rejection, similar to the case of solid liver transplantation.

PHH cultures, established from adult or fetal livers, also represent the most physiologically relevant target cells for HCV infection in vitro. Despite the popularity and success of the cell culture system based on the hepatoma cell line Huh-7 and its derivatives (1999, Lohmann et al., Science 285:110-113; 2002, Blight et al., J Virol. 76:13001-13014), several important aspects of viral infection and host responses cannot be studied in these cell lines. For example, the highly permissive Huh-7.5 cells are defective in RIG-I-mediated interferon production (2005, Sumpter et al., J Virol. 79:2689-2699) and therefore are not suitable for studies of innate immunity against HCV infection. Cell lines outside the Huh-7 series that can support HCV infection have also been reported (2007, Zhu et al., Gastroenterology 133:1649-1659; 2011, Ndongo-Thiam et al., Hepatology 54:406-417; 2011, Long et al., Gastroenterology 141:1057-1066; 2011, Narbus et al., J Virol. 85:12087-12092; 2006, Kanda et al., J Virol. 80:4633-4639). However, in addition to having much lower infection efficiencies, these cells are all derived from tumor tissue or are immortalized, making them incompatible with any research aimed at determining potential effects of viral infection on cancerous transformation. Notwithstanding the importance of PHHs, the usefulness of these cells as a robust culture model for HCV research has been significantly limited by poor accessibility and variability. Procurement of liver biopsy and freshly isolated hepatocytes is difficult for the majority of the labs, and the commercial supplies of PHHs can be unpredictable because of the low plating efficiency of the cells. The variability of PHHs isolated from different patients is another challenge. Differences in patient medical history, host genetics, and methods of isolation all contribute to the difficulty of obtaining reproducible results and comparing data from different labs. For example, Podevin et al. noted that PHH cultures established from patients who had a history of heavy alcohol use were not suitable for infection by HCV produced in cell culture (HCVcc) (2010, Gastroenterology 139:1355-1364). Finally, in studies of interferon-alpha (IFN-α) production in response to HCV infection where experiments cannot be performed with Huh-7.5 cells, special care has to be taken to eliminate the potential co-purification of nonparenchymal cells from liver tissue as those can complicate results regarding the cellular source of IFN-α production (2011, Marukian et al., Hepatology).

The source of HCV particles that can be used in infection studies in cell culture is also limited. The discovery of a HCV genotype 2a genome (JFH-1) that can replicate in cell culture without adaptive mutations (2003, Kato et al., Gastroenterology 125:1808-1817) led to the production of infectious HCVcc particles (2005, Wakita et al., Nat Med. 11:791-796; 2005, Lindenbach et al., Science 309:623-626; 2005, Zhong et al., Proc Natl Acad Sci USA 102:9294-9299; 2005, Cai et al., J Virol. 79:13963-13973), now ubiquitously used in cell culture experiments. These JFH-1 based viruses, along with additional chimeras (2006, Pietschmann et al., Proc Natl Acad Sci USA 103:7408-7413; 2008, Gottwein et al., Hepatology) and a genotype 1a virus that can also produce particles when adaptive mutations were introduced into its genome (2006, Yi et al., Proc Natl Acad Sci USA 103:2310-2315), greatly advanced the cell culture model beyond the subgenomic replicon stage and enabled studies of the full life cycle of HCV. Pietschmann et al. reported that transfection of a genotype 1b full-length genome, either wild-type (wt) or with a replication enhancing mutation in the NS4B protein, into Huh-7.5 cells resulted in secretion of viral particles into the media (2009, Pietschmann et al., PLoS Pathog. 5:e1000475). But demonstration of infectivity of these particles in vitro relied on the inclusion of a kinase inhibitor and efforts to establish a persistent infection culture were unsuccessful. Although a previous study established viral entry into differentiated human hepatocyte-like cells (DHHs) by HIV particles pseudotyped with HCV envelope proteins (HCVpp) (2007, Cai et al., Hepatology 45:1229-1239), productive infection by authentic HCV particles has not previously been reported. Moreover, HCV particles derived from patient serum (HCVser) may differ from HCVcc in important aspects such as buoyant density and virion-associated serum products that are only present in vivo. HCVser infection in vitro has been inefficient and a recent study with the human liver progenitor cell line HepaRG suggests that both immature and mature hepatocyte features may be required efficient infection and replication of HCVser (2011, Ndongo-Thiam et al., Hepatology 54:406-417).

Pluripotent stem cells, either embryonic or induced by reprogramming factors (hESCs and iPSCs, respectively), have the remarkable ability of indefinite self-renewal while maintaining their potential to differentiate into virtually any cell type (1998, Thomson et al., Science 282:1145-1147; 2007, Takahashi et al., Cell 131:861-872), including hepatocyte-like cells (2008, Agarwal et al., Stem Cells 26:1117-1127; 2009, Song et al., Cell Res. 19:1233-1242; 2010, Sullivan et al., Hepatology 51:329-335; 2010, Si-Tayeb et al., Hepatology 51:297-305; 2010, Touboulet al., Hepatology 51:1754-1765; 2010, Ghodsizadeh et al., Stem Cell Rev. 6:622-632; 2010, Rashid et al., J Clin Invest. 120:3127-3136, 2007, Cai et al., Hepatology 45:1229-1239). In vitro differentiated human hepatocyte-like cells (DHHs) express hepatic markers and display hepatic function in vitro. More importantly, DHHs were able to repopulate mice liver and exhibit hepatic function after transplantation in a liver-damaged mouse model (2011, Liu et al., Sci Transl Med. 3, 82ra39).

There remains a great need in the art for compositions and methods for the in vitro culture of hepatitis virus isolates obtained from an infected patient. The present invention addresses this unmet need in the art.

### SUMMARY

The invention relates to the discovery that Differentiated Human Hepatocyte-Like Cells (DHH) derived from stem cells are permissive for infection by a hepatitis virus (HV). In one embodiment, the invention is a method of making a Hepatitis C Virus (HCV)-permissive DHH, including the steps of: contacting at least one stem cell with a first cell culture medium comprising, activin-A, b-FGF and Wnt-3A and incubating the at least one stem cell for about 24 hours; then contacting at least one cell that was contacted with the first cell culture medium with a second cell culture medium comprising, activin-A and b-FGF and incubating the at least one cell for about 3 days; then contacting at least one cell that was contacted with the second cell culture medium with a third cell culture medium comprising FGF-10 and incubating the at least one cell for about 3 days; then contacting at least one cell that was contacted with the third cell culture medium with a fourth cell culture medium comprising, FGF-10, retinoic acid, and SB431542 and incubating the at least one cell for about 3 days. In various embodiments, the stem cell is a pluripotent stem cell. In some embodiments, the pluripotent stem cell is an embryonic stem cell (ESC) or an induced pluripotent stem cell (iPSC). In some embodiments, at least one of the first, second, third, and fourth cell culture medium further comprises at least one of 0-20% Probumin®, 0-2% β-Mercaptoethanol, 0-5% L-Alanyl-L-glutamine, and 0-5% hESC supplement. In some embodiments, the first cell culture medium comprises 1-1000 ng/ml activin-A, 0.1-50 ng/ml b-FGF, and 0.1-1000 ng/ml Wnt-3A. In some embodiments, the second cell culture medium comprises 1-1000 ng/ml activin-A and 0.1-50 ng/ml b-FGF. In some embodiments, the third cell culture medium comprises 0.1-500 ng/ml FGF-10. In some embodiments, the fourth cell culture medium comprises 0.1-500 ng/ml FGF-10, 0.01-10 μM retinoic acid, and 0.1-100 μM SB431542. In one embodiment, the HCV-permissive DHH is permissive for infection by HCVser. In various embodiments, the HCV-permissive DHH is permissive for infection by at least one of the HCV genotypes selected from the group consisting of genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a and 6a. In some embodiments, the method of the invention includes the additional step of contacting at least one cell that was contacted with the first, second, third, and fourth cell culture mediums, with a fifth cell culture medium comprising, FGF-4, EGF, and HGF and incubating the at least one cell from about 1 day to about 10 days. In some embodiments, the fifth culture medium comprises 0.1-100 ng/ml FGF-4, 0.1-1000 ng/ml EGF, and 0.1-1000 ng/ml HGF.

In another embodiment, the invention is a composition comprising an HCV-permissive DHH. In some embodiments, the HCV-permissive DHH is permissive for infection by HCVser. In various embodiments, the HCV-permissive DHH is permissive for infection by at least one of the HCV genotypes selected from the group consisting of genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a and 6a.

In one embodiment, the invention is an HCV culture system comprising an HCV-permissive DHH and an HCV. In some embodiments, the HCV-permissive DHH is permissive for infection by HCVser. In various embodiments, the HCV-permissive DHH is permissive for infection by at least one of the HCV genotypes selected from the group consisting of genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a and 6a. In some embodiments, the HCV is resistant to at least one drug.

In another embodiment, the invention is a method of identifying a test compound as a modulator of HCV infection, including the steps of: placing at least one HCV-permissive DHH in culture medium in a first container; contacting the at least one HCV-permissive DHH in the first container with an HCV in the absence of the test compound; determining the level of HCV in the culture medium in the first container in the absence of the test compound; placing at least one HCV-permissive DHH in culture medium in a second container; contacting the at least one HCV-permissive DHH in the second container with an HCV in the presence of the test compound; determining the level of HCV in the culture medium in the second container in the presence of the test compound; comparing the level of HCV in the presence of the test compound with the level of HCV in the absence of the test compound; identifying the test compound as a modulator of HCV infection when the level of HCV in the presence of the test compound is different than level of HCV in the absence of the test compound. In some embodiments, when the level of HCV is higher in the presence of the test compound, the test compound is identified as an HCV infection activator. In other embodiments, when the level of HCV is lower in the presence of the test compound, the test compound is identified as an HCV infection inhibitor. In some embodiments, the level of HCV is determined by measuring the HCV titer. In other embodiments, the level of HCV is determined by measuring the level of an HCV nucleic acid. In other embodiments, the level of HCV is determined by measuring the level of an HCV polypeptide. In various embodiments, the test compound is at least one selected from the group consisting of: a chemical compound, a protein, a peptide, a peptidomemetic, an antibody, a nucleic acid, an antisense nucleic acid, an shRNA, a ribozyme, and a small molecule chemical compound. In one embodiment, the HCV-permissive DHH is permissive for infection by HCVser. In various embodiments, the HCV-permissive DHH is permissive for infection by at least one of the HCV genotypes selected from the group consisting of genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a and 6a. In some embodiments, the HCV is resistant to at least one drug.

In one embodiment, the invention is a container comprising an HCV-permissive DHH. In one embodiment, the HCV-permissive DHH is permissive for infection by HCVser. In various embodiments, the HCV-permissive DHH is permissive for infection by at least one of the HCV genotypes selected from the group consisting of genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a and 6a.

In another embodiment, the invention is a kit comprising an HCV-permissive DHH and instructional material. In one embodiment, the HCV-permissive DHH is permissive for infection by HCVser. In various embodiments, the HCV-permissive DHH is permissive for infection by at least one of the HCV genotypes selected from the group consisting of genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a and 6a.

In one embodiment, the invention is a method of making a Hepatitis B Virus (HBV)-permissive Differentiated Human Hepatocyte-Like Cell (DHH), the method including the steps of: contacting at least one stem cell with a first cell culture medium comprising, activin-A, b-FGF and Wnt-3A and incubating the at least one stem cell for about 24 hours; then contacting at least one cell that was contacted with the first cell culture medium with a second cell culture medium comprising, activin-A and b-FGF and incubating the at least one cell for about 3 days; then contacting at least one cell that was contacted with the second cell culture medium with a third cell culture medium comprising FGF-10 and incubating the at least one cell for about 3 days; then contacting at least one cell that was contacted with the third cell culture medium with a fourth cell culture medium comprising, FGF-10, retinoic acid, and SB431542 and incubating the at least one cell for about 3 days. In some embodiments, the stem cell is a pluripotent stem cell. In some embodiments, the pluripotent stem cell is an embryonic stem cell (ESC) or an induced pluripotent stem cell (iPSC). In some embodiments, at least one of the first, second, third, and fourth cell culture medium further comprises at least one of 0-20% Probumin®, 0-2% β-Mercaptoethanol, 0-5% L-Alanyl-L-glutamine, and 0-5% hESC supplement. In one embodiment, the first cell culture medium comprises 1-1000 ng/ml activin-A, 0.1-50 ng/ml b-FGF, and 0.1-1000 ng/ml Wnt-3A. In one embodiment, the second cell culture medium comprises 1-1000 ng/ml activin-A and 0.1-50 ng/ml b-FGF. In one embodiment, the third cell culture medium comprises 0.1-500 ng/ml FGF-10. In one embodiment, the fourth cell culture medium comprises 0.1-500 ng/ml FGF-10, 0.01-10 μM retinoic acid, and 0.1-100 μM SB431542. In some embodiments, the HBV-permissive DHH is permissive for infection by HBVser. In various embodiments, the HBV-permissive DHH is permissive for infection by at least one of the HBV genotypes selected from the group consisting of genotype A, B, C, D, E, F, G and H. In some embodiments, the method includes the additional step of contacting at least one cell that was contacted with the first, second, third, and fourth cell culture mediums, with a fifth cell culture medium comprising, FGF-4, EGF, and HGF and incubating the at least one cell from about 1 day to about 10 days. In some embodiments, the fifth culture medium comprises 0.1-100 ng/ml FGF-4, 0.1-1000 ng/ml EGF, and 0.1-1000 ng/ml HGF.

In another embodiment, the invention is a composition comprising an HBV-permissive DHH. In one embodiment, the HBV-permissive DHH is permissive for infection by HBVser. In various embodiments, the HBV-permissive DHH is permissive for infection by at least one of the HBV genotypes selected from the group consisting of genotype A, B, C, D, E, F, G and H.

In one embodiment, the invention is an HBV culture system comprising an HBV-permissive DHH and an HBV. In some embodiments, the HBV-permissive DHH is permissive for infection by HBVser. In various embodiments, the HBV-permissive DHH is permissive for infection by at least one of the HBV genotypes selected from the group consisting of genotype A, B, C, D, E, F, G and H. In some embodiments, the HBV is resistant to at least one drug.

In another embodiment, the invention is a method of identifying a test compound as a modulator of HBV infection, including the steps of: placing at least one HBV-permissive DHH in culture medium in a first container; contacting the at least one HBV-permissive DHH in the first container with an HBV in the absence of the test compound; determining the level of HBV in the culture medium in the first container in the absence of the test compound; placing at least one HBV-permissive DHH in culture medium in a second container; contacting the at least one HBV-permissive DHH in the second container with an HBV in the presence of the test compound; determining the level of HBV in the culture medium in the second container in the presence of the test compound; comparing the level of HBV in the presence of the test compound with the level of HBV in the absence of the test compound; identifying the test compound as a modulator of HBV infection when the level of HBV in the presence of the test compound is different than level of HBV in the absence of the test compound. In some embodiments, when the level of HBV is higher in the presence of the test compound, the test compound is identified as an HBV infection activator. In other embodiments, when the level of HBV is lower in the presence of the test compound, the test compound is identified as an HBV infection inhibitor. In some embodiments, the level of HBV is determined by measuring the HBV titer. In other embodiments, the level of HBV is determined by measuring the level of an HBV nucleic acid. In other embodiments, the level of HBV is determined by measuring the level of an HBV polypeptide. In various embodiments, the test compound is at least one selected from the group consisting of: a chemical compound, a protein, a peptide, a peptidomemetic, an antibody, a nucleic acid, an antisense nucleic acid, an shRNA, a ribozyme, and a small molecule chemical compound. In some embodiments, the HBV-permissive DHH is permissive for infection by HBVser. In various embodiments, the HBV-permissive DHH is permissive for infection by at least one of the HBV genotypes selected from the group consisting of genotype A, B, C, D, E, F, G and H. In one embodiment, the HBV is resistant to at least one drug.

In one embodiment, the invention is a container comprising an HBV-permissive DHH. In one embodiment, the HBV-permissive DHH is permissive for infection by HBVser. In various embodiments, the HBV-permissive DHH is permissive for infection by at least one of the HBV genotypes selected from the group consisting of genotype A, B, C, D, E, F, G and H.

In another embodiment, the invention is a kit comprising an HBV-permissive DHH and instructional material. In one embodiment, the HBV-permissive DHH is permissive for infection by HBVser. In various embodiments, the HBV-permissive DHH is permissive for infection by at least one of the HBV genotypes selected from the group consisting of genotype A, B, C, D, E, F, G and H.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts the results of experiments examining hepatic differentiation from hESCs. (A) Representative images of cell morphology and protein marker expression of hESCs (day 0), definitive endoderm (day 4), hepatic progenitor cells (days 8-10), and hepatocyte-like cells (both immature and mature, days 11-21). For day-10 cells, double-staining of AFP and CK-7 (middle panel, 40×) showed mutually exclusive expression in the cell population. (B) Albumin secretion by DHHs. Culture media were collected at the indicated time points during differentiation and subjected to Albumin detection with an ELISA kit. Error bars represent standard deviation from replicate experiments. (C) Reciprocal expression of pluripotent marker Nanog and liver-specific marker AFP during differentiation.

FIG. 2 depicts the results of experiments assessing infection of DHHs derived from hESCs and iPSCs. (A) Detection of HCV proteins in DHHs infected with JFH-1 based HCVcc. DHHs were inoculated with three different preparations of HCVcc at day 13 post-differentiation and cell lysates collected at day 21 for western blot analysis. The anti-NS3 antibody also recognized a non-specific band in the mock infected sample. (B) Immunostaining of infected DHHs. A JFH variant containing a FLAG tag in the NS5A protein was used to infect either Huh-7.5 or DHHs and staining was done with an anti-FLAG antibody. (C) Infection of Huh-7.5 and DHHs as measured by the HCV-dependent fluorescence relocalization assay. Reporter-transduced cells were infected with HCVcc and the cells were fixed for immunofluorescence analysis 72 hours post-infection. For the RFP-NLS-IPS expressing cells, HCV infection leads to complete nuclear translocation of the RFP; for the EGFP-IPS cells, HCV infection leads to redistribution of green fluorescence from a reticulate cytoplasmic pattern to a diffused pattern with a nuclear enrichment. (D) HCV inhibitors abolished HCV infection in DHHs. The following inhibitors were included in the infection experiments. IFN: interferon-α, 80 units/ml; AR3A: anti-E2 neutralizing antibody, 1 μg/ml; ITX: ITX5061, an SR-BI inhibitor, 1 μM. (E) Comparison of HCVcc infection levels in DHHs and PHHs. PHHs were infected for 8 days to keep the infected days the same between the two cell types (DHHs were infected at day 13 and the lysed at day 21). (F) Infection of DHHs derived from an iPSC line. Differentiation and infection of iPS.K3 were performed as described for H9-derived DHHs (FIG. 1 and FIG. 2A).

FIG. 3 depicts the results of experiments showing that DHHs support persistent infection and secrete infectious HCV particles. (A) Continuous replication of HCVcc in DHHs. Day-10 DHHs were infected with Jc1/GLuc2A for 9 hours before the inoculum was removed and the cells were changed into medium E with or without a cyclophilin inhibitor CsA at 1 μg/ml. Culture supernatants were collected daily to measure luciferase activity. The culture media was replaced with thorough washing every 48 hours and CsA was included every time fresh media was used. Error bars represent standard deviation from triplicate experiments. (B) Secretion of HCV core antigen into the culture medium by infected DHHs. Day-13 DHHs were infected with HCVcc for 9 hours before the inoculum was removed and the cells washed and changed into media E, which were immediately collected as the 0 hour samples. The infected cells were then incubated for an additional 48 hours in media E with or without IFN-α (50 units/ml) before the culture supernatants were collected as the 48 hour samples. Error bars represent standard deviation from replicate experiments. (C) Re-infection of Huh-7.5 cells by HCV particles produced from DHHs. The 48 hour media from (B) were used to infect Huh-7.5 cells which were then fixed for NS3 staining four days post-infection.

FIG. 4 depicts the results of experiments assessing the time course of infection to determine the transition point at which the differentiating cells become permissive for HCV. (A) An example time course of cell culture media. (B) Cells were infected for 6 hours at the indicated days before the inoculum was removed. The cells were then cultured in the appropriate medium for an additional 48 hours before the cell lysates were collected for detection of NS3 expression. (C) Secreted luciferase activities were monitored in the same experiments described in (B). Error bars represent standard deviation of triplicate experiments. (D) Hepatic maturation was not required for HCV infection of day-10 cells. Day-10 DHHs were infected and the either kept in medium D (hepatic specification medium, HSM) or changed into HGF-containing medium E (hepatic maturation medium) until day 21, when all cells were collected for western blotting. The anti-NS3 antibody also recognized a non-specific band in the mock infected sample. (E) A diagram indicating the time point for transition of DHHs to HCV-permissiveness based on results shown in (A) and (B).

FIG. 5 depicts the results of experiments assessing the cellular determinants of HCV susceptibility. (A) Induction of miR-122 expression by FGF-10 during hepatic specification. Equal amounts of total cellular RNA for various cells as indicated were subjected to a real-time RT-PCR assay for detection of miR-122 expression. (B) Microarray heat map of gene expression levels in day-10 versus day-7 cells. Two independent RNA samples were processed from day-7 and day-10 cells. The numbers represent the average values and standard deviations. The conventional color spectrum with representing downregulation and representing upregulation was adopted. (C) Quantitative RT-PCR results of EGFR and EphA2 expression induction. (D) Upregulation of PI4KIIIα protein during the differentiation process. The levels of CyPA and DDX-3 remained unchanged in the same samples.

FIG. 6 depicts the results of experiments performing genetic modification of hESCs and HCV-resistant DHHs. (A) Suppression of CyPA expression by shRNA in H9 cells and day-21 DHHs. (B) CyPA knockdown did not affect the expression of pluripotency marker Oct-4 in H9 cells. (C) Modified DHHs were resistant to HCV infection. Infection of both the wild-type (wt) and CyPA-KD (LA) DHHs were done at day 13 and allowed to proceed for 48 hours. Luciferase in the culture supernatant for monitored. Wt HCVcc (Jc1/GLuc2A) infected unmodified DHHs but not CyPA-KD DHHs (redlines) and the DEYN mutant infected both cell types (blue lines). Error bars represent standard deviation of replicate experiments.

FIG. 7 depicts the results of experiments assessing the direct infection of DHHs by HCV derived from patient serum. (A) Detection of IFN-sensitive HCVser infection by western blotting IFN-α was included in the medium at 50 units/ml when indicated. (B) Visualization of single cell infection events by HCVser using the HCV-dependent fluorescence relocalization assay. Arrows indicate individual cells infected with HCVser and showing nuclear translocation of the RFP. (C) Secretion of HCV core antigen into culture supernatant by HCVser-infected DHHs. Values for core levels in supernatants collected 48 hours post-infection were plotted. IFN-α was included in the medium at 50 units/ml when indicated. Error bars represent standard deviation of replicate experiments. (D) HCVser preferentially infected DHHs over Huh-7.5 cells. Equal amounts of genome equivalent of HCVser were used to infect either Huh-7.5 or day-11 DHHs. Core levels in the supernatants collected at 0 and 48 hours were plotted for both cell lines. Error bars represent standard deviation of replicate experiments.

FIG. 8 depicts the results of experiments assessing the expression of HCV cofactors during the hepatic differentiation process. (A) Microarray heat map of expression levels of reported HCV cofactors in day-10 versus day-7 cells. (B) Expression profile of HCV cofactors as represented by conventional RT-PCR and gel analysis.

FIG. 9 depicts the results of experiments assessing the expression of surface receptors during the hepatic differentiation process. (A) Cell surface staining of the four well characterized receptors (CD81, SR-BI, Claudin-1, and Occludin) for HCV entry in both H9 and day-10 cells. (B) RT-PCR analysis of receptor expression during the hepatic differentiation process.

FIG. 10 depicts the results of experiments assessing PI4KIII knockdown in DHHs. (A) Suppression of PI4KIIIα by shRNA in Huh-7.5 cells. (B) PI4KIIIα KD efficiently blocked HCV infection in Huh-7.5 cells. (C) PI4KIIIα KD in H9 cells. (D) DHHs with PI4KIIIα KD were resistant to HCV infection. The infections were done at day 13 and the luciferase activity was monitored for the next 48 hours. Error bars represent standard deviation of replicate experiments.

FIG. 11 depicts the results of experiments measuring infection efficiency of DHHs cultured in three-dimensional scaffolds. For the 3-D cultures, day-9 cells were seeded on to either polystyrene or polycaprolactone scaffolds, which were transferred to a new dish after adherence of the cells. Infections by Jc1/GLuc2A were performed at day 13 and luciferase assays were done in the next two days. The luciferase results were normalized to the cell numbers and then compared with those of the regular (2-D) cultures, which were set to be 100%. Error bars represent standard deviation of replicate experiments.

FIG. 12 is a schematic depicting an exemplary differentiation scheme for making HV-permissive DHH derived from a stem cell.

FIG. 13, comprising FIGS. 13A-13D, is a list of genes that increased in their level of expression (and their fold change) after cells were placed into Medium D.

FIG. 14, comprising FIGS. 14A-14B, is a list of genes that decreased in their level of expression (and their fold change) after cells were placed into Medium D.

### DETAILED DESCRIPTION

The invention relates to the discovery that differentiated human hepatocyte-like cells (DHH) derived from stem cells are permissive for infection by a hepatitis virus (HV). In various embodiments, the HV is at least one of hepatitis A virus (HAV), hepatitis B virus (HBV), hepatitis C virus (HCV), hepatitis D virus (HDV) and hepatitis E virus (HEV). In one embodiment, the invention includes an HV-permissive DHH. In another embodiment, the invention includes a method of making an HV-permissive DHH derived from a stem cell. In one embodiment, the invention includes an HV culture system comprising at least one HV-permissive DHH. In various embodiments, the invention includes a method of using the HV-permissive DHH or HV culture system to conduct HV life cycle analyses, to diagnose a subject as being infected with HV, to genotype and characterize the HV of a subject infected with HV, to detect drug resistance of HV obtained from a subject infected with HV, to screen for and identify modulators of HV infection, and to monitor the effect of a treatment of HV in a subject, among other uses of the invention.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, to “alleviate” a disease means to reduce the frequency or severity of at least one sign or symptom of a disease or disorder.

As used herein the terms “alteration,” “defect,” “variation,” or “mutation,” refers to a mutation in a gene in a cell that affects the function, activity, expression (transcription or translation) or conformation of the polypeptide that it encodes. Mutations encompassed by the present invention can be any mutation of a nucleic acid that results in the enhancement or disruption of the function, activity, expression or conformation of the encoded polypeptide, including the complete absence of expression of the encoded protein and can include, for example, missense and nonsense mutations, insertions, deletions, frameshifts and premature terminations. Without being so limited, mutations encompassed by the present invention may alter splicing of RNA or cause a shift in the reading frame (frameshift).

The term “amplification” refers to the operation by which the number of copies of a target nucleotide sequence present in a sample is multiplied.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. The antibodies in the present invention may exist in a variety of forms including, for example, polyclonal antibodies, monoclonal antibodies, intracellular antibodies (“intrabodies”), Fv, Fab and F(ab)2, as well as single chain antibodies (scFv), heavy chain antibodies, such as camelid antibodies, synthetic antibodies, chimeric antibodies, and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “coding sequence,” as used herein, means a sequence of a nucleic acid or its complement, or a part thereof, that can be transcribed and/or translated to produce the mRNA and/or the polypeptide or a fragment thereof. Coding sequences include exons in a genomic DNA or immature primary RNA transcripts, which are joined together by the cell's biochemical machinery to provide a mature mRNA. The anti-sense strand is the complement of such a nucleic acid, and the coding sequence can be deduced therefrom. In contrast, the term “non-coding sequence,” as used herein, means a sequence of a nucleic acid or its complement, or a part thereof, that is not translated into amino acid in vivo, or where tRNA does not interact to place or attempt to place an amino acid. Non-coding sequences include both intron sequences in genomic DNA or immature primary RNA transcripts, and gene-associated sequences such as promoters, enhancers, silencers, and the like.

As used herein, the terms “complementary” or “complementarity” are used in reference to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods that depend upon binding between nucleic acids.

As used herein, the term “diagnosis” refers to the determination of the presence of a disease or disorder. In some embodiments of the present invention, methods for making a diagnosis are provided which permit determination of the presence of a particular disease or disorder.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between).

The term “gene” refers to a nucleic acid (e.g., DNA) sequence that includes coding sequences necessary for the production of a polypeptide, precursor, or RNA (e.g., mRNA). The polypeptide may be encoded by a full length coding sequence or by any portion of the coding sequence so long as the desired activity or functional property (e.g., enzymatic activity, ligand binding, signal transduction, immunogenicity, etc.) of the full-length or fragment is retained. The term also encompasses the coding region of a structural gene and the sequences located adjacent to the coding region on both the 5′ and 3′ ends for a distance of about 2 kb or more on either end such that the gene corresponds to the length of the full-length mRNA and 5′ regulatory sequences which influence the transcriptional properties of the gene. Sequences located 5′ of the coding region and present on the mRNA are referred to as 5′-untranslated sequences. The 5′-untranslated sequences usually contain the regulatory sequences. Sequences located 3′ or downstream of the coding region and present on the mRNA are referred to as 3′-untranslated sequences. The term “gene” encompasses RNA, cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids. A single molecule that contains pairing of complementary nucleic acids within its structure is said to be “self-hybridized.” A single DNA molecule with internal complementarity could assume a variety of secondary structures including loops, kinks or, for long stretches of base pairs, coils.

As used herein, an “immunoassay” refers to any binding assay that uses an antibody capable of binding specifically to a target molecule to detect and quantify the target molecule.

As used herein, an “instructional material” includes a publication, a recording, a diagram, or any other medium of expression which can be used to communicate the usefulness of a compound, composition, method, virus, vector, or system of the invention in the kit for practicing the methods described herein. The instructional material of the kit of the invention can, for example, be affixed to a container which contains the identified compound, composition, vector, or delivery system of the invention or be shipped together with a container which contains the identified compound, composition, method components, virus, vector, or system of the invention. Alternatively, the instructional material can be shipped separately from the container with the intention that the instructional material and the compound be used cooperatively by the recipient.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

The term “label” when used herein refers to a detectable compound or composition that is conjugated directly or indirectly to a probe to generate a “labeled” probe. The label may be detectable by itself (e.g. radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

The terms “microarray” and “array” refers broadly to “DNA microarrays,” “DNA chip(s),” “protein microarrays” and “protein chip(s)” and encompasses all art-recognized solid supports, and all art-recognized methods for affixing nucleic acid, peptide, and polypeptide molecules thereto. Preferred arrays typically comprise a plurality of different nucleic acid or peptide probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 5,800,992, 6,040,193, 5,424,186 and Fodor et al., 1991, Science, 251:767-777, each of which is incorporated by reference in its entirety for all purposes. Arrays may generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array may be fabricated on a surface of virtually any shape or even a multiplicity of surfaces. Arrays may be nucleic acids on beads, gels, polymeric surfaces, fibers such as fiber optics, glass or any other appropriate substrate. (See U.S. Pat. Nos. 5,770,358, 5,789,162, 5,708,153, 6,040,193 and 5,800,992, which are hereby incorporated by reference in their entirety for all purposes.) Arrays may be packaged in such a manner as to allow for diagnostic use or can be an all-inclusive device; e.g., U.S. Pat. Nos. 5,856,174 and 5,922,591 incorporated in their entirety by reference for all purposes. Arrays are commercially available from, for example, Affymetrix (Santa Clara, Calif.) and Applied Biosystems (Foster City, Calif.), and are directed to a variety of purposes, including genotyping, diagnostics, mutation analysis, marker expression, and gene expression monitoring for a variety of eukaryotic and prokaryotic organisms. The number of probes on a solid support may be varied by changing the size of the individual features. In one embodiment the feature size is 20 by 25 microns square, in other embodiments features may be, for example, 8 by 8, 5 by 5 or 3 by 3 microns square, resulting in about 2,600,000, 6,600,000 or 18,000,000 individual probe features.

Assays for amplification of the known sequence are also disclosed. For example primers for PCR may be designed to amplify regions of the sequence. For RNA, a first reverse transcriptase step may be used to generate double stranded DNA from the single stranded RNA. The array may be designed to detect sequences from an entire genome; or one or more regions of a genome, for example, selected regions of a genome such as those coding for a protein or RNA of interest; or a conserved region from multiple genomes; or multiple genomes, arrays and methods of genetic analysis using arrays is described in Cutler, et al., 2001, Genome Res. 11(11): 1913-1925 and Warrington, et al., 2002, Hum Mutat 19:402-409 and in US Patent Pub No 20030124539, each of which is incorporated herein by reference in its entirety.

By the term “modulating,” as used herein, is meant mediating a detectable increase or decrease in the level of a nucleic acid, polypeptide, and/or viral titer in the presence of a treatment or compound, compared with the level of a nucleic acid, polypeptide, and/or viral titer, in the absence of the treatment or compound. The term encompasses perturbing and/or affecting a signal or response, thereby mediating a beneficial therapeutic response in a subject, preferably, a human.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferably at least 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”.

The terms “patient,” “subject,” “individual,” and the like are used interchangeably herein, and refer to any animal amenable to the methods described herein. In certain non-limiting embodiments, the patient, subject or individual is a human.

As used herein, the term “polymerase chain reaction” (“PCR”) refers to the method of K. B. Mullis (U.S. Pat. Nos. 4,683,195 4,683,202, and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. As used herein, the terms “PCR product,” “PCR fragment,” “amplification product” or “amplicon” refer to the resultant mixture of compounds after two or more cycles of the PCR steps of denaturation, annealing and extension are complete. These terms encompass the case where there has been amplification of one or more segments of one or more target sequences.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “polynucleotide” includes cDNA, RNA, DNA/RNA hybrid, antisense RNA, small-hairpin RNA (shRNA), ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

The term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are cofactors or which affect conditions such as pH and the like at various suitable temperatures. A primer is preferably a single strand sequence, such that amplification efficiency is optimized, but double stranded sequences can be utilized.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

“Sample” or “biological sample” as used herein means a biological material isolated from a subject or from in vitro culture. The biological sample may contain any biological material suitable for detecting a nucleic acid, polypeptide or other marker of a biologic, physiologic or pathologic process in a subject or in vitro cell culture, and may comprise culture media, body fluid, tissue, and cellular and/or non-cellular material obtained from a subject or in vitro cell culture.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

As used herein, “substantially purified” refers to being essentially free of other components. For example, a substantially purified cell is a cell which has been separated from other cell types with which it is normally associated in its naturally occurring state. In some instances, a population of substantially purified cells refers to a homogenous population of cells. In other instances, this term refers simply to a cell that has been separated from the cells with which they are naturally associated in their natural state.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Targets are sometimes referred to in the art as anti-probes. As the term target as used herein, no difference in meaning is intended.

As used herein, the terms “therapy” or “therapeutic regimen” refer to those activities taken to alleviate or alter a disorder or disease state, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

To “treat” a disease as the term is used herein, means to reduce the frequency or severity of at least one sign or symptom of a disease or disorder experienced by a subject.

As used herein, the term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “mutant” refers to a gene or gene product that displays modifications in sequence and/or functional properties (i.e., altered characteristics; e.g., drug resistance) when compared to the wild-type gene or gene product. It is noted that naturally occurring mutants can be isolated; these are identified by the fact that they have altered characteristics (including altered nucleic acid sequences) when compared to the wild-type gene or gene product.

### DESCRIPTION

The invention relates to the discovery that DHH derived from stem cells are permissive for infection by a hepatitis virus (HV). In various embodiments of the invention described herein, the HV is at least one of hepatitis A virus (HAV), hepatitis B virus (HBV), hepatitis C virus (HCV), hepatitis D virus (HDV) and hepatitis E virus (HEV).

### HCV

The invention relates to the discovery that DHH derived from stem cells are permissive for infection by both HCVcc and HCVser. In various embodiments, the DHH derived from stem cells are permissive for infection by HCV of at least one genotype selected from the group consisting of genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a or 6a. In some embodiments, the DHH derived from stem cells are permissive for infection by HCV that is resistant to at least one drug.

In one embodiment, the invention includes an HCV-permissive DHH. In another embodiment, the invention includes an HCV culture system comprising at least one HCV-permissive DHH. In various embodiments, the invention includes a method of using the HCV-permissive DHH or HCV culture system to conduct HCV life cycle analyses, to diagnose a subject as being infected with HCV, to genotype and characterize the HCV of a subject infected with HCV, to detect drug resistance of HCV obtained from a subject infected with HCV, to screen for and identify modulators of HCV infection, and to monitor the effect of a treatment of HCV in a subject.

### HCV-Permissive Cells and Methods of Making HCV-Permissive Cells

In one embodiment, the invention includes an HCV-permissive DHH. In various embodiments, the DHH is derived from stem cell. In various other embodiments, the stem cell is a pluripotent stem cell. In one embodiment, the stem cell is an embryonic stem cell (ESC). In another embodiment, the stem cell is a human pluripotent stem cell (hESC). In a further embodiment, the stem cell is an induce pluripotent stem cell (iPSC).

In one embodiment, the invention includes a method of making an HCV-permissive DHH from a stem cell. In various embodiments, the stem cell is a pluripotent stem cell. In one embodiment, the stem cell is an embryonic stem cell (ESC). In another embodiment, the stem cell is a human embryonic stem cell (hESC). In a further embodiment, the stem cell is an induce pluripotent stem cell (iPSC). Examples of stem cells useful in the methods of the invention include, but are not limited to, those described in Takahashi and Yamanaka, 2006, Cell 126:663-76; Zhou et al., 2009, Cell Stem Cell 4:381-384; Okita et al., 2007, Nature 448:313-317; Wernig et al., 2007, Nature 448:318-324; Yu et al., 2007, Science 318:1917-1920; Takahashi et al., 2007, Cell 131:861-872; Okita et al., 2008, Science 322:949-953; Thomson et al., 1998, Science 282:1145-1147; Andrews, 2005, Biochem Soc Trans 33:1526-30; Mountford, 2008, Transfus Med 18: 1112; Amit et al., 2000, Developmental Biology 227:271-278; Odorico et al., 2001, Stem Cells 19:193-204; and Human Embryonic Stem Cells, 2007, S. Sullivan, C. Cowan, K. Eggan (eds.), John Wiley & Sons, Ltd.

In one embodiment, the invention includes a method of making an HCV-permissive DHH, derived from a stem cell. In various embodiments, the method of making an HCV-permissive DHH, derived from a stem cell, comprises a multi-step method of exposing a stem cell to series of chemicals over about a 10-20 day period. In various embodiments, prior to differentiation to an HCV-permissive DHH, stem cells are cultured in a suitable stem cell culture medium. One non-limiting example of a suitable stem cell culture medium is Stempro® (Invitrogen, Carlsbad Calif.).

For Step 1 of the method of making an HCV-permissive DHH, derived from a stem cell, the media the stem cells are bathed in is changed to DMEM/F12 (Invitrogen, Carlsbad Calif.), comprising 0-20% Probumin (Millipore, Billerica, Mass.), 0-2% β-Mercaptoethanol, 0-5% L-Alanyl-L-glutamine, 0-5% hESC supplement, 1-1000 ng/ml activin-A, 0.1-50 ng/ml b-FGF (also known as FGF-2 and FGF-β, and 0.1-1000 ng/ml Wnt-3A) and the cells are incubated for about 18-36 hours. For Step 2, the media the cells are bathed in is changed to DMEM/F12 (Invitrogen, Carlsbad Calif.), comprising 0-20% Probumin (Millipore, Billerica, Mass.), 0-2% β-Mercaptoethanol, 0-5% L-Alanyl-L-glutamine, 0-5% hESC supplement, 1-1000 ng/ml activin-A and 0.1-50 ng/ml b-FGF (also known as FGF-2 and FGF-β and the cells are incubated for about 2-4 days. For Step 3, the media the cells are bathed in is changed to DMEM/F12 (Invitrogen, Carlsbad Calif.), comprising 0-20% Probumin (Millipore, Billerica, Mass.), 0-2% β-Mercaptoethanol, 0-5% L-Alanyl-L-glutamine, 0-5% hESC supplement, and 0.1-500 ng/ml FGF-10 and the cells are incubated for about 2-4 days. For Step 4, the media the cells are bathed in is changed to DMEM/F12 (Invitrogen, Carlsbad Calif.), comprising 0-20% Probumin (Millipore, Billerica, Mass.), 0-2% β-Mercaptoethanol, 0-5% L-Alanyl-L-glutamine, 0-5% hESC supplement, 0.1-500 ng/ml FGF-10, 0.01-10 μM retinoic acid, and 0.1-100 μM SB431542 and the cells are incubated for about 2-4 days. For Step 5, media the cells are bathed in is changed to DMEM/F12 (Invitrogen, Carlsbad Calif.), comprising 0-20% Probumin (Millipore, Billerica, Mass.), 0-2% β-Mercaptoethanol, 0-5% L-Alanyl-L-glutamine, 0-5% hESC supplement, 0.1-100 ng/ml FGF-4, 0.1-1000 ng/ml EGF, and 0.1-1000 ng/ml HGF and the cells are incubated from about 1 day to about 10-15 days, replacing the media with fresh media every two or three days.

In a particular embodiment of the method of making an HCV-permissive DHH, prior to differentiation, stem cells are cultured in a suitable stem cell culture medium. One non-limiting example of a suitable stem cell culture medium is Stempro® (Invitrogen, Carlsbad Calif.). For Step 1 of the method of making an HCV-permissive DHH, the media the stem cells are bathed in is changed to DMEM/F12 (Invitrogen, Carlsbad Calif.), comprising 20% Probumin (Millipore, Billerica, Mass.), 2% 3-Mercaptoethanol, 5% L-Alanyl-L-glutamine, 5% hESC supplement, 100 ng/ml activin-A, 8 ng/ml b-FGF (also known as FGF-2 and FGF-β, and 25 ng/ml Wnt-3A) and the cells are incubated for about 24 hours. For Step 2, the media the cells are bathed in is changed to DMEM/F12 (Invitrogen, Carlsbad Calif.), comprising 20% Probumin (Millipore, Billerica, Mass.), 2% β-Mercaptoethanol, 5% L-Alanyl-L-glutamine, 5% hESC supplement, 100 ng/ml activin-A and 8 ng/ml b-FGF (also known as FGF-2 and FGF-β and the cells are incubated for about 3 days. For Step 3, the media the cells are bathed in is changed to DMEM/F12 (Invitrogen, Carlsbad Calif.), comprising 20% Probumin (Millipore, Billerica, Mass.), 2% β-Mercaptoethanol, 5% L-Alanyl-L-glutamine, 5% hESC supplement, and 50 ng/ml FGF-10 and the cells are incubated for about 3 days. For Step 4, the media the cells are bathed in is changed to DMEM/F12 (Invitrogen, Carlsbad Calif.), comprising 20% Probumin (Millipore, Billerica, Mass.), 2% β-Mercaptoethanol, 5% L-Alanyl-L-glutamine, 5% hESC supplement, 50 ng/ml FGF-10, 0.1 μM retinoic acid, and 1 μM SB431542 and the cells are incubated for about 3 days. For Step 5, media the cells are bathed in is changed to DMEM/F12 (Invitrogen, Carlsbad Calif.), comprising 20% Probumin (Millipore, Billerica, Mass.), 2% β-Mercaptoethanol, 5% L-Alanyl-L-glutamine, 5% hESC supplement, 30 ng/ml FGF-4, 50 ng/ml EGF, and 50 ng/ml HGF and the cells are incubated from about 1 day to about 10-15 days, replacing the media with fresh media every two or three days.

The skilled artisan will understand that many hESC supplements are known in the art that can be used in the media described herein. By way of one non-limiting example, a suitable hESC supplement for use with the methods described herein can be made as described here. In some embodiments, 1 mL of hESC supplement is made by mixing together: 50 μl trace elements A (e.g., Cat #: 99-182-CI, Cellgro), 50 μl trace elements B (e.g., Cat #: 99-176-CI, Cellgro), 50 μl trace elements C (e.g, Cat #: 99-175-CI, Cellgro), 500 μl non-essential amino acids (100×), 2.5 mg L-Ascorbic acid, 125 μl L-Glutamine (100×), 100 mg Probumin (e.g., Cat #: 810683, Millipore), 500 μg Bovine or Human Transferrin (e.g., Invitrogen), and DMEM/F12 (e.g., Cat #: 15-090-CM, Cellgro) up to a final volume of 1 mL. By way of another non-limiting example, a suitable hESC supplement for use with the methods described herein can be obtained, for example, from the Stem Cell Core Facility at the University of Georgia operating under NIH Grant No. 5P01GM085354.

### HCV Culture System and Methods of Use

In one embodiment, the invention includes an HCV culture system comprising at least one HCV-permissive DHH. In various embodiments described elsewhere herein, the invention includes a method of using the HCV-permissive DHH in the HCV culture system of the invention to conduct HCV life cycle analyses, to diagnose a subject as being infected with HCV, to genotype and characterize the HCV of a subject infected with HCV, to detect drug resistance of HCV isolate obtained from a subject infected with HCV, to screen for and identify modulators of HCV infection, and to monitor the effect of a treatment of HCV in a subject.

In one embodiment, the HCV culture system of the invention comprises at least one HCV and at least one HCV-permissive DHH cultured in a suitable media. One non-limiting example of a suitable media is DMEM/F12 (Invitrogen, Carlsbad Calif.), comprising 0-20% Probumin (Millipore, Billerica, Mass.), 0-2% β-Mercaptoethanol, 0-5% L-Alanyl-L-glutamine, 0-5% hESC supplement, 0.1-100 ng/ml FGF-10, 0.01-10 μM retinoic acid, and 0.1-10 μM SB431542. Another non-limiting example of a suitable media is DMEM/F12 (Invitrogen, Carlsbad Calif.), comprising 0-20% Probumin (Millipore, Billerica, Mass.), 0-2% β-Mercaptoethanol, 0-5% L-Alanyl-L-glutamine, 0-5% hESC supplement, 0.1-100 ng/ml FGF-4, 0.1-500 ng/ml EGF, and 0.1-500 ng/ml HGF.

In various embodiments, the DHH of the HCV culture system of the invention were derived from a stem cell. In one embodiment, the stem cell is a pluripotent stem cell. In another embodiment, the stem cell is an embryonic stem cell (ESC). In yet another embodiment, the stem cell is a human pluripotent stem cell (hESC). In a further embodiment, the stem cell is an induce pluripotent stem cell (iPSC). Examples of stem cells useful in the methods of the invention include, but are not limited to, those described in Takahashi and Yamanaka, 2006, Cell 126:663-76; Zhou et al., 2009, Cell Stem Cell 4:381-384; Okita et al., 2007, Nature 448:313-317; Wernig et al., 2007, Nature 448:318-324; Yu et al., 2007, Science 318:1917-1920; Takahashi et al., 2007, Cell 131:861-872; Okita et al., 2008, Science 322:949-953; Thomson et al., 1998, Science 282:1145-1147; Andrews, 2005, Biochem Soc Trans 33:1526-30; Mountford, 2008, Transfus Med 18: 1112; Amit et al., 2000, Developmental Biology 227:271-278; Odorico et al., 2001, Stem Cells 19:193-204; and Human Embryonic Stem Cells, 2007, S. Sullivan, C. Cowan, K. Eggan (eds.), John Wiley & Sons, Ltd.

In various embodiments, the HCV of the HCV culture system of the invention includes of at least one genotype selected from the group consisting of genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a or 6a. In some embodiments, the HCV of the HCV culture system is resistant to at least one drug.

The HCV culture system of the invention can include any kind of substrate, surface, scaffold or container known in the art useful for culturing cells or for culturing virus. Non-limiting examples of such containers include cell culture plates, dishes and flasks. Other suitable substrates, surfaces and containers are described in Culture of Animal Cells: a manual of basic techniques (3rd edition), 1994, R. I. Freshney (ed.), Wiley-Liss, Inc.; Cells: a laboratory manual (vol. 1), 1998, D. L. Spector, R. D. Goldman, L. A. Leinwand (eds.), Cold Spring Harbor Laboratory Press; Embryonic Stem Cells, 2007, J. R. Masters, B. O. Palsson and J. A. Thomson (eds.), Springer; Stem Cell Culture, 2008, J. P. Mather (ed.) Elsevier; and Animal Cells: culture and media, 1994, D. C. Darling, S. J. Morgan John Wiley and Sons, Ltd. In some embodiments, the HCV culture system comprises a two-dimensional scaffold. In other embodiments, the HCV culture system comprises a three-dimensional scaffold. Non-limiting examples of three-dimensional culture systems useful in the invention are described in Kinney et al. (2012, Integr Biol (Camb) 4:641-50) and Ploss et al., 2012, Proc Natl Acad Sci USA 107:3141-5).

In one embodiment, the HCV culture system described herein is used to determine the genotype of the HCV obtained from an infected subject. Knowing the genotype of the HCV infecting a subject is useful for determining which treatment regimens are best suited for a particular subject, as different genotypes may be more or less susceptible to particular therapeutic regimens. The culture system described herein is useful for determining the genotype of HCV infecting a particular subject, because the DHH of the invention are permissive to infection by HCVser. Briefly, the serum from an HCV infected subject can be used to infect DHH in the HCV culture system of the infection, the HCV can be harvested from the cells or culture media of the HCV culture system of the invention, and the genotype of the HCV can be determined using methods known in the art.

In another embodiment, the HCV culture system described herein is used to determine whether the HCV obtained from an infected subject is resistant to a particular drug or class of drugs. Knowing whether the HCV infecting a subject is resistant to a particular drug or class of drugs is useful for determining which treatment regimens are best suited for that subject. In some embodiments, the drug resistance status of the HCV obtained from a subject is determined by culturing the HCV obtained from the subject in the HCV cell culture system of the invention, and determining whether the nucleic acid of the HCV obtained from the subject has a mutation known to be associated with resistance to a particular drug or class of drug. In other embodiments, the drug resistance status of the HCV obtained from a subject is determined by culturing the HCV obtained from the subject in the HCV cell culture system of the invention, in the presence and absence of an anti-HCV drug, and assessing whether the presence of the drug in the culture interferes with the HCV infection.

In another embodiment, the HCV culture system described herein is used to characterize the life cycle of HCV obtained from an infected subject. The HCV culture system of the inventions is useful for culturing HCVcc and HCVser of any HCV genotype. Having an in vitro HCV culture system as described herein, able to HCVcc and HCVser of any HCV genotype, provides unique opportunities for studying and characterizing the critical HCV components, host cell components, and HCV component-host cell component interactions that could not previously been studied. In various embodiments, the HCV culture system of the invention can be used in an assay to identify and characterize a receptor, a co-receptor, an HCV component, and a host cell component involved in the HCV life cycle.

### HCV Methods of Diagnosis, Prognosis, Therapy Selection and Therapy Evaluation

The present invention also provides methods of diagnosing a subject as being infected with HCV. Further, the invention provides methods of assessing the prognosis of a subject infected with HCV, as well as methods of monitoring the effectiveness of a treatment administered to a subject infected with HCV.

In one embodiment, the method of the invention comprises a diagnostic assay for diagnosing HCV infection in a subject in need thereof. In one non-limiting example, an HCV-permissive DHH is contacted with a biological sample obtained from the subject and cultured as described herein. To make a diagnosis of HCV infection, the presence or absence or the level of the HCV titer, an HCV protein, an HCV nucleic acid, or a combination thereof, is assessed and compared with the level of at least one comparator control, such as a positive control, a negative control, a historical control, or a historical norm. The presence or absence or the level of the HCV titer, an HCV protein, an HCV nucleic acid, or a combination thereof, is assessed 1 day, 2 days, 3 days, 4 days, 5 days, 6 days, 7 days, 8 days, 9 days, 10 days, or more days after the DHH was contacted with the biological sample obtained from the subject. The presence or absence or the level of the HCV titer, an HCV protein, an HCV nucleic acid, or a combination thereof, can be assessed in cell culture media, in cells, or in combinations there. In one embodiment, the biological sample is serum. In another embodiment, the biological sample is HCVser.

In a further embodiment, the method of the invention comprises an assay for monitoring the effectiveness of an HCV treatment administered to a subject in need thereof. The method includes determining whether the level of HCV in a biological sample obtained from the subject is modulated upon administration of the treatment. The assay can be performed before, during or after a treatment has been administered, or any combination thereof. In this method, an HCV-permissive DHH is contacted with a biological sample obtained from the subject and cultured as elsewhere described herein. The presence or absence or level of the HCV titer, an HCV protein, an HCV nucleic acid, or a combination thereof, is assessed and compared with the level of at least one comparator control, such as a pre-treatment sample, a prior post-treatment sample, a positive control, a negative control, a historical control, or a historical norm. The presence or absence or the level of the HCV titer, an HCV protein, an HCV nucleic acid, or a combination thereof, is assessed 1 day, 2 days, 3 days, 4 days, 5 days, 6 days, 7 days, 8 days, 9 days, 10 days, or more days after the DHH was contacted with the biological sample obtained from the subject. The presence or absence or the level of the HCV titer, an HCV protein, an HCV nucleic acid, or a combination thereof, can be assessed in cell culture media, in cells, or in combinations there. In one embodiment, the biological sample is serum. In another embodiment, the biological sample is HCVser. Comparing the presence or absence or the level of the HCV titer, an HCV protein, an HCV nucleic acid, or a combination thereof, before treatment and after treatment, indicates whether the administered treatment is modulating the infection. When the level of the HCV titer, an HCV protein, an HCV nucleic acid, or a combination thereof, in the biological sample is decreased after administration of the treatment, the treatment is having a therapeutic effect on the infection.

In various embodiments, the level of HCV is assessed by measuring at least a fragment of an HCV polypeptide or an HCV nucleic acid. The term, “fragment,” as used herein, indicates that the portion of the polypeptide, mRNA or cDNA is of a length that is sufficient to identify the fragment as a fragment of an HCV polypeptide or an HCV nucleic acid.

The biological sample obtained from the subject can be a sample from any source which potentially contains virus, such as a body fluid or a tissue, or a combination thereof. A biological sample can be obtained by appropriate methods, such as, by way of examples, blood draw, fluid draw, or biopsy. A biological sample can be used directly, or can be processed, and the processed biological sample can then be used as the test sample.

The culture media assessed for the level of HCV titer, HCV polypeptide, or HCV nucleic acid can be assessed directly, or can be processed to enhance access to the HCV polypeptide or HCV nucleic acid. Alternatively or in addition, if desired, an amplification method can be used to amplify nucleic acids comprising all or a fragment of a nucleic acid in a test sample.

In various embodiments of the invention, methods of measuring an HCV polypeptide level include, but are not limited to, an immunochromatography assay, an immunodot assay, a Luminex assay, an ELISA assay, an ELISPOT assay, a protein microarray assay, a ligand-receptor binding assay, displacement of a ligand from a receptor assay, an immunostaining assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, a FACS assay, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

In some embodiments, quantitative hybridization methods, such as Southern analysis, Northern analysis, or in situ hybridizations, can be used (see Current Protocols in Molecular Biology, Ausubel, F. et al., eds., John Wiley & Sons, including all supplements). A “nucleic acid probe,” as used herein, can be a DNA probe or an RNA probe. The probe can be, for example, a gene, a gene fragment (e.g., one or more exons), a vector comprising the gene, a probe or primer, etc. For representative examples of use of nucleic acid probes, see, for example, U.S. Pat. Nos. 5,288,611 and 4,851,330. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate target nucleic acid. The hybridization sample is maintained under conditions which are sufficient to allow specific hybridization of the nucleic acid probe to the nucleic acid target. Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe having a target nucleic acid in the test sample, the level of the target nucleic acid in the sample can be assessed. More than one nucleic acid probe can also be used concurrently in this method. Specific hybridization of any one of the nucleic acid probes is indicative of the presence of the target nucleic acid, as described herein.

Alternatively, a peptide nucleic acid (PNA) probe can be used instead of a nucleic acid probe in the quantitative hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a target nucleic acid sequence. Hybridization of the PNA probe to a nucleic acid sequence is used to determine the level of the target nucleic acid in the sample.

In another embodiment, arrays of oligonucleotide probes that are complementary to target nucleic acid sequences in the biological sample obtained from a subject can be used to determine the level of nucleic acid in the sample. The array of oligonucleotide probes can be used to determine the level of the target nucleic acid alone, or the level of the target nucleic acid in relation to the level of one or more other nucleic acids in the sample. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a nucleic acid of interest is hybridized with the array and its level is quantified. Hybridization and quantification are generally carried out by methods described herein and also in, e.g., published PCT Application Nos. WO 92/10092 and WO 95/11995, and U.S. Pat. No. 5,424,186. In brief, a target nucleic acid sequence is amplified by well-known amplification techniques, e.g., PCR. Typically, this involves the use of primer sequences that are complementary to the target nucleic acid. Asymmetric PCR techniques may also be used. Amplified target, generally incorporating a label, is then hybridized with the array under appropriate conditions. Upon completion of hybridization and washing of the array, the array is scanned to determine the quantity of hybridized nucleic acid. The hybridization data obtained from the scan is typically in the form of fluorescence intensities as a function of quantity, or relative quantity, of the target nucleic acid in the biological sample. The target nucleic acid can be hybridized to the array in combination with one or more comparator controls (e.g., positive control, negative control, quantity control, etc.) to improve quantification of the target nucleic acid in the sample.

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention includes carried out with radioactive elements or with nonradioactive molecules. Among the radioactive isotopes used, mention may be made of 32P, 33P, 35S or 3H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents.

Nucleic acids can be obtained from culture media or from cells using known techniques. Nucleic acid herein refers to RNA, including mRNA, and DNA, including cDNA. The nucleic acid can be double-stranded or single-stranded (i.e., a sense or an antisense single strand) and can be complementary to a nucleic acid encoding a polypeptide. The nucleic acid content may also be an RNA or DNA extraction performed on a sample, including a culture media sample, biological fluid and fresh or fixed tissue sample.

There are many methods known in the art for the detection and quantification of specific nucleic acid sequences and new methods are continually reported. A great majority of the known specific nucleic acid detection and quantification methods utilize nucleic acid probes in specific hybridization reactions. Preferably, the detection of hybridization to the duplex form is a Southern blot technique. In the Southern blot technique, a nucleic acid sample is separated in an agarose gel based on size (molecular weight) and affixed to a membrane, denatured, and exposed to (admixed with) the labeled nucleic acid probe under hybridizing conditions. If the labeled nucleic acid probe forms a hybrid with the nucleic acid on the blot, the label is bound to the membrane.

In the Southern blot, the nucleic acid probe is preferably labeled with a tag. That tag can be a radioactive isotope, a fluorescent dye or the other well-known materials. Another type of process for the specific detection of nucleic acids in a biological sample known in the art are the hybridization methods as exemplified by U.S. Pat. No. 6,159,693 and No. 6,270,974, and related patents. To briefly summarize one of those methods, a nucleic acid probe of at least 10 nucleotides, preferably at least 15 nucleotides, more preferably at least 25 nucleotides, having a sequence complementary to a nucleic acid of interest is hybridized in a sample, subjected to depolymerizing conditions, and the sample is treated with an ATP/luciferase system, which will luminesce if the nucleic sequence is present. In quantitative Southern blotting, the level of the nucleic acid of interest can be compared with the level of a second nucleic acid of interest, and/or to one or more comparator control nucleic acids (e.g., positive control, negative control, quantity control, etc.).

Many methods useful for the detection and quantification of nucleic acid takes advantage of the polymerase chain reaction (PCR). The PCR process is well known in the art (U.S. Pat. No. 4,683,195, No. 4,683,202, and No. 4,800,159). To briefly summarize PCR, nucleic acid primers, complementary to opposite strands of a nucleic acid amplification target sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as discussed elsewhere herein. Most preferably the detection of the duplex is done using at least one primer directed to the nucleic acid of interest. In yet another embodiment of PCR, the detection of the hybridized duplex comprises electrophoretic gel separation followed by dye-based visualization.

Typical hybridization and washing stringency conditions depend in part on the size (i.e., number of nucleotides in length) of the oligonucleotide probe, the base composition and monovalent and divalent cation concentrations (Ausubel et al., 1994, eds Current Protocols in Molecular Biology).

In a preferred embodiment, the process for determining the quantitative and qualitative profile of the nucleic acid of interest according to the present invention includes characterized in that the amplifications are real-time amplifications performed using a labeled probe, preferably a labeled hydrolysis-probe, capable of specifically hybridizing in stringent conditions with a segment of the nucleic acid of interest. The labeled probe is capable of emitting a detectable signal every time each amplification cycle occurs, allowing the signal obtained for each cycle to be measured.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market, and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present invention includes the Tm, which is in the range of about 65° C. to 75° C. Preferably, the Tm for any one of the hydrolysis-probes of the present invention includes in the range of about 67° C. to about 70° C. Most preferably, the Tm applied for any one of the hydrolysis-probes of the present invention is about 67° C.

In one aspect, the invention includes a primer that is complementary to a nucleic acid of interest, and more particularly the primer includes 12 or more contiguous nucleotides substantially complementary to the nucleic acid of interest. Preferably, a primer featured in the invention includes a nucleotide sequence sufficiently complementary to hybridize to a nucleic acid sequence of about 12 to 25 nucleotides. More preferably, the primer differs by no more than 1, 2, or 3 nucleotides from the target flanking nucleotide sequence In another aspect, the length of the primer can vary in length, preferably about 15 to 28 nucleotides in length (e.g., 16, 17, 18, 19, 20, 21, 22, 23, 24, 25, 26, or 27 nucleotides in length).

### Methods of Identifying a Modulator of HCV Infection

The current invention relates to a method of identifying a compound that modulates HCV infection. In some embodiments, the method of identifying of the invention identifies an HCV infection inhibitor compound that diminishes HCV infection in the HCV culture system of the invention. In other embodiments, the method of identifying of the invention identifies an HCV infection activator compound that increases HCV infection in the HCV culture system of the invention. In various embodiments, the level of HCV infection can be assessed by measuring the level of an HCV protein, or by measuring the level of an HCV nucleic acid, or a combination thereof. The invention further comprises compositions comprising the modulator of HCV infection, identified by the methods described herein.

In one embodiment, the invention comprises a method of identifying a test compound as a modulator of HCV infection. Generally, the method of identifying a test compound as a modulator of HCV infection includes comparing a parameter of HCV infection in the presence of a test compound with a parameter of HCV infection in the absence of the test compound. Thus, in some embodiments, the method includes the steps of: placing at least one HCV-permissive DHH in culture medium in a first container, contacting the at least one HCV-permissive DHH in the first container with an HCV in the absence of the test compound, determining the level of HCV in the culture medium in the first container in the absence of the test compound; and placing at least one HCV-permissive DHH in culture medium in a second container, contacting the at least one HCV-permissive DHH in the second container with an HCV in the presence of the test compound, determining the level of HCV in the culture medium in the second container in the presence of the test compound; and comparing the level of HCV in the presence of the test compound with the level of HCV in the absence of the test compound; and identifying the test compound as a modulator of HCV infection when the level of HCV in the presence of the test compound is different than level of HCV in the absence of the test compound. In one embodiment, when the level of HCV is higher in the presence of the test compound, the test compound is identified as an HCV infection activator. In another embodiment, when the level of HCV is lower in the presence of the test compound, the test compound is identified as an HCV infection inhibitor. In various embodiments, the level of HCV is determined by measuring the HCV titer, an HCV nucleic acid, an HCV polypeptide, and combinations thereof. Suitable test compounds include, but are not limited to, a chemical compound, a protein, a peptide, a peptidomemetic, an antibody, a nucleic acid, an antisense nucleic acid, an shRNA, a ribozyme, and a small molecule chemical compound.

In one embodiment, the HCV-permissive DHH is permissive for infection by HCVcc. In another embodiment, the HCV-permissive DHH is permissive for infection by HCVser. In a further embodiment, the HCV-permissive DHH is permissive for infection by both HCVcc and HCVser. In various embodiments, the HCV-permissive DHH is permissive for infection by at least one of the HCV genotypes selected from the group consisting of genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a and 6a. In a particular embodiment, the HCV-permissive DHH is permissive for infection by HCV that is resistant to at least one drug.

Other methods, as well as variation of the methods disclosed herein will be apparent from the description of this invention. In various embodiments, the test compound concentration in the screening assay can be fixed or varied. A single test compound, or a plurality of test compounds, can be tested at one time. Suitable test compounds that may be used include, but are not limited to, proteins, nucleic acids, antisense nucleic acids, small molecules, antibodies and peptides.

The invention relates to a method for screening test compounds to identify a modulator compound by its ability to modulate the level of HCV infection in the HCV culture system of the invention, by measuring HCV infection parameters in the presence and absence of the test compound.

The test compounds can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam et al., 1997, Anticancer Drug Des. 12:45).

Examples of methods for the synthesis of molecular libraries can be found in the art, for example, in: DeWitt et al., 1993, Proc. Natl. Acad. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (Ladner U.S. Pat. No. 5,223,409), spores (Ladner U.S. Pat. No. '409), plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; Felici, 1991, J. Mol. Biol. 222:301-310; and Ladner supra).

In situations where “high-throughput” modalities are preferred, it is typical that new chemical entities with useful properties are generated by identifying a chemical compound (called a “lead compound”) with some desirable property or activity, creating variants of the lead compound, and evaluating the property and activity of those variant compounds.

In one embodiment, high throughput screening methods involve providing a library containing a large number of test compounds potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

### Genetically-Modified HCV-Permissive and Non-Permissive Cells

The invention also includes genetically-modified HCV-permissive and non-permissive DHH. In one embodiment, the stem cell used to derive an HCV-permissive DHH, using the methods described elsewhere herein, is genetically modified. In another embodiment, the stem cell used to derive an HCV-non-permissive DHH, using the methods described elsewhere herein, is genetically modified.

In one embodiment, the HCV-non-permissive DHH is derived from a genetically-modified stem cell possessing a genetic modification rendering the stem cell, and its DHH progeny, resistant to infection by HCV. In various embodiments, the genetic modification reduces or eliminates a host cell component necessary to render the cell permissive to HCV infection. Non-limiting examples of host cell components that can be reduced or eliminated to render the cell non-permissive to HCV infection include receptors and co-receptors. In a one embodiment, the genetic modification results in the reduction or elimination of cyclophilin A in the DHH. In another embodiment, the genetic modification results in the reduction or elimination of PI4KIIIα in the DHH. In a particular embodiment, stem cells isolated from a subject are genetically-modified and used to derive HCV-non-permissive DHH that are transplanted into the same, or a different, subject, to populate the subject with a population of HCV-non-permissive DHH.

The stem cells may be genetically modified using any method known to the skilled artisan. See, for instance, Sambrook et al. (2001, Molecular Cloning: A Laboratory Manual, Cold Spring Harbor Laboratory Press, Cold Spring Harbor, N.Y.), and in Ausubel et al,. Eds, (1997, Current Protocols in Molecular Biology, John Wiley & Sons, New York, N.Y.). For example, a stem cell may be exposed to an expression vector comprising a nucleic acid including a transgene, such that the nucleic acid is introduced into the cell under conditions appropriate for the transgene to be expressed within the cell. The transgene generally is an expression cassette, including a polynucleotide operably linked to a suitable promoter. The polynucleotide can encode a protein, or it can encode biologically active RNA (e.g., antisense RNA, shRNA, or a ribozyme). Thus, for example, the polynucleotide can encode a gene conferring resistance HCV infection.

Within the expression cassette, the coding polynucleotide is operably linked to a suitable promoter. Examples of suitable promoters include prokaryotic promoters and viral promoters (e.g., retroviral LTRs, lentiviral LTRs, immediate early viral promoters (IEp), such as herpesvirus IEp (e.g., ICP4-IEp and ICP0-IEEp), cytomegalovirus (CMV) IEp, and other viral promoters, such as Rous Sarcoma Virus (RSV) promoters, and Murine Leukemia Virus (MLV) promoters). Other suitable promoters are eukaryotic promoters or enhancers (e.g., the rabbit (β-globin regulatory elements), constitutively active promoters (e.g., the (β-actin promoter, etc.), signal specific promoters (e.g., inducible promoters such as a promoter responsive to RU486, etc.), and tissue-specific promoters (e.g., liver-specific promoter). It is well within the skill of the art to select a promoter suitable for driving gene expression in a predefined cellular context. The expression cassette can include more than one coding polynucleotide, and it can include other elements (e.g., polyadenylation sequences, sequences encoding a membrane-insertion signal or a secretion leader, ribosome entry sequences, transcriptional regulatory elements (e.g., enhancers, silencers, etc.), and the like), as desired.

The expression cassette containing the transgene should be incorporated into a genetic vector suitable for delivering the transgene to the cells. Depending on the desired end application, any such vector can be so employed to genetically modify the cells (e.g., plasmids, naked DNA, viruses such as adenovirus, adeno-associated virus, herpesviruses, lentiviruses, papillomaviruses, retroviruses, etc.). Any method of constructing the desired expression cassette within such vectors can be employed, many of which are well known in the art (e.g., direct cloning, homologous recombination, etc.). The choice of vector will largely determine the method used to introduce the vector into the cells (e.g., by protoplast fusion, calcium-phosphate precipitation, gene gun, electroporation, DEAE dextran or lipid carrier mediated transfection, infection with viral vectors, etc.), which are generally known in the art.

Examples of techniques sufficient to direct persons of skill through in vitro amplification methods, including the polymerase chain reaction (PCR), the ligase chain reaction (LCR), and other DNA or RNA polymerase-mediated techniques are found in Sambrook et al., Molecular Cloning: A Laboratory Manual, volumes 1-3 (3rd ed., Cold Spring Harbor Press, NY 2001).

Once the nucleic acid for a protein is cloned, a skilled artisan may express the recombinant gene(s) in a variety of stem and liver cells. It is expected that those of skill in the art are knowledgeable in the numerous expression systems available for expressing the desired transgene.

### HCV Kits

The present invention also pertains to kits useful in the methods of the invention. Such kits comprise various combinations of components useful in any of the methods described elsewhere herein, including for example, hybridization probes or primers (e.g., labeled probes or primers), antibodies, reagents for detection of labeled molecules, materials for the amplification of nucleic acids, a stem cell, components for deriving an HCV-permissive DHH from a stem cell, an HCV-permissive DHH cell, materials for quantitatively analyzing an HCV polypeptide and/or an HCV nucleic acid, and instructional material. For example, in one embodiment, the kit comprises components useful for deriving an HCV-permissive DHH from a stem cell.

In one embodiment, the kit comprises the components of a diagnostic assay for diagnosing HCV infection in a subject in need thereof, containing instructional material and the components for determining the level of HCV in a biological sample obtained from the subject, the genotype of HCV in a biological sample obtained from a subject, and/or the drug resistance status of HCV in a biological sample obtained from a subject. In various embodiments, determining the level, genotype or drug resistance status of HCV in a biological sample obtained from the subject requires a comparison to at least one comparator control contained in the kit, such as a positive control, a negative control, a historical control, a historical norm, or the level of another reference molecule in the biological sample.

In a further embodiment, the kit comprises the components of an assay for monitoring the effectiveness of a treatment administered to a subject in need thereof, containing instructional material and the components for determining the level of HCV in a biological sample obtained from the subject, the genotype of HCV in a biological sample obtained from a subject, and/or the drug resistance status of HCV in a biological sample obtained from a subject. In various embodiments, determining the level, genotype or drug resistance status of HCV in a biological sample obtained from the subject requires a comparison to at least one comparator control contained in the kit, such as a positive control, a negative control, a historical control, a historical norm, or the level of another reference molecule in the biological sample.

### HBV

In another embodiment, the invention includes a DHH derived from a stem cell that is permissive for infection by HBVcc or HBVser. In various embodiments, the DHH derived from stem cells are permissive for infection by HBV of at least one genotype selected from the group consisting of genotype A, B, C, D, E, F, G and H. In some embodiments, the DHH derived from stem cells are permissive for infection by HBV that is resistant to at least one drug.

In one embodiment, the invention includes an HBV-permissive DHH. In another embodiment, the invention includes an HBV culture system comprising at least one HBV-permissive DHH. In various embodiments, the invention includes a method of using the HBV-permissive DHH or HBV culture system to conduct HBV life cycle analyses, to diagnose a subject as being infected with HBV, to genotype and characterize the HBV of a subject infected with HBV, to detect drug resistance of HBV obtained from a subject infected with HBV, to screen for and identify modulators of HBV infection, and to monitor the effect of a treatment of HBV in a subject.

### HBV-Permissive Cells and Methods of Making HBV-Permissive Cells

In one embodiment, the invention includes an HBV-permissive DHH. In various embodiments, the DHH is derived from stem cell. In various other embodiments, the stem cell is a pluripotent stem cell. In one embodiment, the stem cell is an embryonic stem cell (ESC). In another embodiment, the stem cell is a human pluripotent stem cell (hESC). In a further embodiment, the stem cell is an induce pluripotent stem cell (iPSC).

In one embodiment, the invention includes a method of making an HBV-permissive DHH from a stem cell. In various embodiments, the stem cell is a pluripotent stem cell. In one embodiment, the stem cell is an embryonic stem cell (ESC). In another embodiment, the stem cell is a human embryonic stem cell (hESC). In a further embodiment, the stem cell is an induce pluripotent stem cell (iPSC). Examples of stem cells useful in the methods of the invention include, but are not limited to, those described in Takahashi and Yamanaka, 2006, Cell 126:663-76; Zhou et al., 2009, Cell Stem Cell 4:381-384; Okita et al., 2007, Nature 448:313-317; Wernig et al., 2007, Nature 448:318-324; Yu et al., 2007, Science 318:1917-1920; Takahashi et al., 2007, Cell 131:861-872; Okita et al., 2008, Science 322:949-953; Thomson et al., 1998, Science 282:1145-1147; Andrews, 2005, Biochem Soc Trans 33:1526-30; Mountford, 2008, Transfus Med 18: 1112; Amit et al., 2000, Developmental Biology 227:271-278; Odorico et al., 2001, Stem Cells 19:193-204; and Human Embryonic Stem Cells, 2007, S. Sullivan, C. Cowan, K. Eggan (eds.), John Wiley & Sons, Ltd.

In one embodiment, the invention includes a method of making an HBV-permissive DHH, derived from a stem cell. In various embodiments, the method of making an HBV-permissive DHH, derived from a stem cell, comprises a multi-step method of exposing a stem cell to series of chemicals over about a 10-20 day period. In various embodiments, prior to differentiation to an HBV-permissive DHH, stem cells are cultured in a suitable stem cell culture medium. One non-limiting example of a suitable stem cell culture medium is Stempro® (Invitrogen, Carlsbad Calif.).

For Step 1 of the method of making an HBV-permissive DHH, derived from a stem cell, the media the stem cells are bathed in is changed to DMEM/F12 (Invitrogen, Carlsbad Calif.), comprising 0-20% Probumin (Millipore, Billerica, Mass.), 0-2% β-Mercaptoethanol, 0-5% L-Alanyl-L-glutamine, 0-5% hESC supplement, 1-1000 ng/ml activin-A, 0.1-50 ng/ml b-FGF (also known as FGF-2 and FGF-β, and 0.1-1000 ng/ml Wnt-3A) and the cells are incubated for about 18-36 hours. For Step 2, the media the cells are bathed in is changed to DMEM/F12 (Invitrogen, Carlsbad Calif.), comprising 0-20% Probumin (Millipore, Billerica, Mass.), 0-2% β-Mercaptoethanol, 0-5% L-Alanyl-L-glutamine, 0-5% hESC supplement, 1-1000 ng/ml activin-A and 0.1-50 ng/ml b-FGF (also known as FGF-2 and FGF-β) and the cells are incubated for about 2-4 days. For Step 3, the media the cells are bathed in is changed to DMEM/F12 (Invitrogen, Carlsbad Calif.), comprising 0-20% Probumin (Millipore, Billerica, Mass.), 0-2% β-Mercaptoethanol, 0-5% L-Alanyl-L-glutamine, 0-5% hESC supplement, and 0.1-500 ng/ml FGF-10 and the cells are incubated for about 2-4 days. For Step 4, the media the cells are bathed in is changed to DMEM/F12 (Invitrogen, Carlsbad Calif.), comprising 0-20% Probumin (Millipore, Billerica, Mass.), 0-2% β-Mercaptoethanol, 0-5% L-Alanyl-L-glutamine, 0-5% hESC supplement, 0.1-500 ng/ml FGF-10, 0.01-10 μM retinoic acid, and 0.1-100 μM SB431542 and the cells are incubated for about 2-4 days. For Step 5, media the cells are bathed in is changed to DMEM/F12 (Invitrogen, Carlsbad Calif.), comprising 0-20% Probumin (Millipore, Billerica, Mass.), 0-2% β-Mercaptoethanol, 0-5% L-Alanyl-L-glutamine, 0-5% hESC supplement, 0.1-100 ng/ml FGF-4, 0.1-1000 ng/ml EGF, and 0.1-1000 ng/ml HGF and the cells are incubated from about 1 day to about 10-15 days, replacing the media with fresh media every two or three days.

In a particular embodiment of the method of making an HBV-permissive DHH, prior to differentiation, stem cells are cultured in a suitable stem cell culture medium. One non-limiting example of a suitable stem cell culture medium is Stempro® (Invitrogen, Carlsbad Calif.). For Step 1 of the method of making an HBV-permissive DHH, the media the stem cells are bathed in is changed to DMEM/F12 (Invitrogen, Carlsbad Calif.), comprising 20% Probumin (Millipore, Billerica, Mass.), 2% 3-Mercaptoethanol, 5% L-Alanyl-L-glutamine, 5% hESC supplement, 100 ng/ml activin-A, 8 ng/ml b-FGF (also known as FGF-2 and FGF-β), and 25 ng/ml Wnt-3A) and the cells are incubated for about 24 hours. For Step 2, the media the cells are bathed in is changed to DMEM/F12 (Invitrogen, Carlsbad Calif.), comprising 20% Probumin (Millipore, Billerica, Mass.), 2% β-Mercaptoethanol, 5% L-Alanyl-L-glutamine, 5% hESC supplement, 100 ng/ml activin-A and 8 ng/ml b-FGF (also known as FGF-2 and FGF-β and the cells are incubated for about 3 days. For Step 3, the media the cells are bathed in is changed to DMEM/F12 (Invitrogen, Carlsbad Calif.), comprising 20% Probumin (Millipore, Billerica, Mass.), 2% β-Mercaptoethanol, 5% L-Alanyl-L-glutamine, 5% hESC supplement, and 50 ng/ml FGF-10 and the cells are incubated for about 3 days. For Step 4, the media the cells are bathed in is changed to DMEM/F12 (Invitrogen, Carlsbad Calif.), comprising 20% Probumin (Millipore, Billerica, Mass.), 2% β-Mercaptoethanol, 5% L-Alanyl-L-glutamine, 5% hESC supplement, 50 ng/ml FGF-10, 0.1 μM retinoic acid, and 1 μM SB431542 and the cells are incubated for about 3 days. For Step 5, media the cells are bathed in is changed to DMEM/F12 (Invitrogen, Carlsbad Calif.), comprising 20% Probumin (Millipore, Billerica, Mass.), 2% β-Mercaptoethanol, 5% L-Alanyl-L-glutamine, 5% hESC supplement, 30 ng/ml FGF-4, 50 ng/ml EGF, and 50 ng/ml HGF and the cells are incubated from about 1 day to about 10-15 days, replacing the media with fresh media every two or three days.

The skilled artisan will understand that many hESC supplements are known in the art that can be used in the media described herein. By way of one non-limiting example, a suitable hESC supplement for use with the methods described herein can be made as described here. In some embodiments, 1 mL of hESC supplement is made by mixing together: 50 μl trace elements A (e.g., Cat #: 99-182-CI, Cellgro), 50 μl trace elements B (e.g., Cat #: 99-176-CI, Cellgro), 50 μl trace elements C (e.g, Cat #: 99-175-CI, Cellgro), 500 μl non-essential amino acids (100×), 2.5 mg L-Ascorbic acid, 125 μl L-Glutamine (100×), 100 mg Probumin (e.g., Cat #: 810683, Millipore), 500 μg Bovine or Human Transferrin (e.g., Invitrogen), and DMEM/F12 (e.g., Cat #: 15-090-CM, Cellgro) up to a final volume of 1 mL By way of another non-limiting example, a suitable hESC supplement for use with the methods described herein can be obtained, for example, from the Stem Cell Core Facility at the University of Georgia operating under NIH Grant No. 5P01GM085354.

### HBV Culture System and Methods of Use

In one embodiment, the invention includes an HBV culture system comprising at least one HBV-permissive DHH. In various embodiments described elsewhere herein, the invention includes a method of using the HBV-permissive DHH in the HBV culture system of the invention to conduct HBV life cycle analyses, to diagnose a subject as being infected with HBV, to genotype and characterize the HBV of a subject infected with HBV, to detect drug resistance of HBV isolate obtained from a subject infected with HBV, to screen for and identify modulators of HBV infection, and to monitor the effect of a treatment of HBV in a subject.

In one embodiment, the HBV culture system of the invention comprises at least one HBV and at least one HBV-permissive DHH cultured in a suitable media. One non-limiting example of a suitable media is DMEM/F12 (Invitrogen, Carlsbad Calif.), comprising 0-20% Probumin (Millipore, Billerica, Mass.), 0-2% β-Mercaptoethanol, 0-5% L-Alanyl-L-glutamine, 0-5% hESC supplement, 0.1-100 ng/ml FGF-10, 0.01-10 μM retinoic acid, and 0.1-10 μM SB431542. Another non-limiting example of a suitable media is DMEM/F12 (Invitrogen, Carlsbad Calif.), comprising 0-20% Probumin (Millipore, Billerica, Mass.), 0-2% β-Mercaptoethanol, 0-5% L-Alanyl-L-glutamine, 0-5% hESC supplement, 0.1-100 ng/ml FGF-4, 0.1-500 ng/ml EGF, and 0.1-500 ng/ml HGF.

In various embodiments, the DHH of the HBV culture system of the invention were derived from a stem cell. In one embodiment, the stem cell is a pluripotent stem cell. In another embodiment, the stem cell is an embryonic stem cell (ESC). In yet another embodiment, the stem cell is a human pluripotent stem cell (hESC). In a further embodiment, the stem cell is an induce pluripotent stem cell (iPSC). Examples of stem cells useful in the methods of the invention include, but are not limited to, those described in Takahashi and Yamanaka, 2006, Cell 126:663-76; Zhou et al., 2009, Cell Stem Cell 4:381-384; Okita et al., 2007, Nature 448:313-317; Wernig et al., 2007, Nature 448:318-324; Yu et al., 2007, Science 318:1917-1920; Takahashi et al., 2007, Cell 131:861-872; Okita et al., 2008, Science 322:949-953; Thomson et al., 1998, Science 282:1145-1147; Andrews, 2005, Biochem Soc Trans 33:1526-30; Mountford, 2008, Transfus Med 18: 1112; Amit et al., 2000, Developmental Biology 227:271-278; Odorico et al., 2001, Stem Cells 19:193-204; and Human Embryonic Stem Cells, 2007, S. Sullivan, C. Cowan, K. Eggan (eds.), John Wiley & Sons, Ltd.

In various embodiments, the HBV of the HBV culture system of the invention includes of at least one genotype selected from the group consisting of genotype A, B, C, D, E, F, G and H. In some embodiments, the HBV of the HBV culture system is resistant to at least one drug.

The HBV culture system of the invention can include any kind of substrate, surface, scaffold or container known in the art useful for culturing cells or for culturing virus. Non-limiting examples of such containers include cell culture plates, dishes and flasks. Other suitable substrates, surfaces and containers are described in Culture of Animal Cells: a manual of basic techniques (3rd edition), 1994, R. I. Freshney (ed.), Wiley-Liss, Inc.; Cells: a laboratory manual (vol. 1), 1998, D. L. Spector, R. D. Goldman, L. A. Leinwand (eds.), Cold Spring Harbor Laboratory Press; Embryonic Stem Cells, 2007, J. R. Masters, B. O. Palsson and J. A. Thomson (eds.), Springer; Stem Cell Culture, 2008, J. P. Mather (ed.) Elsevier; and Animal Cells: culture and media, 1994, D. C. Darling, S. J. Morgan John Wiley and Sons, Ltd. In some embodiments, the HBV culture system comprises a two-dimensional scaffold. In other embodiments, the HBV culture system comprises a three-dimensional scaffold.

In one embodiment, the HBV culture system described herein is used to determine the genotype of the HBV obtained from an infected subject. Knowing the genotype of the HBV infecting a subject is useful for determining which treatment regimens are best suited for a particular subject, as different genotypes may be more or less susceptible to particular therapeutic regimens. The culture system described herein is useful for determining the genotype of HBV infecting a particular subject, because the DHH of the invention are permissive to infection by HBVser. Briefly, the serum from an HBV infected subject can be used to infect DHH in the HBV culture system of the infection, the HBV can be harvested from the cells or culture media of the HBV culture system of the invention, and the genotype of the HBV can be determined using methods known in the art.

In another embodiment, the HBV culture system described herein is used to determine whether the HBV obtained from an infected subject is resistant to a particular drug or class of drugs. Knowing whether the HBV infecting a subject is resistant to a particular drug or class of drugs is useful for determining which treatment regimens are best suited for that subject. In some embodiments, the drug resistance status of the HBV obtained from a subject is determined by culturing the HBV obtained from the subject in the HBV cell culture system of the invention, and determining whether the nucleic acid of the HBV obtained from the subject has a mutation known to be associated with resistance to a particular drug or class of drug. In other embodiments, the drug resistance status of the HBV obtained from a subject is determined by culturing the HBV obtained from the subject in the HBV cell culture system of the invention, in the presence and absence of an anti-HBV drug, and assessing whether the presence of the drug in the culture interferes with the HBV infection.

In another embodiment, the HBV culture system described herein is used to characterize the life cycle of HBV obtained from an infected subject. The HBV culture system of the inventions is useful for culturing HBVcc and HBVser of any HBV genotype. Having an in vitro HBV culture system as described herein, able to HBVcc and HBVser of any HBV genotype, provides unique opportunities for studying and characterizing the critical HBV components, host cell components, and HBV component-host cell component interactions that could not previously been studied. In various embodiments, the HBV culture system of the invention can be used in an assay to identify and characterize a receptor, a co-receptor, an HBV component, and a host cell component involved in the HBV life cycle.

### HBV Methods of Diagnosis, Prognosis, Therapy Selection and Therapy Evaluation

The present invention also provides methods of diagnosing a subject as being infected with HBV. Further, the invention provides methods of assessing the prognosis of a subject infected with HBV, as well as methods of monitoring the effectiveness of a treatment administered to a subject infected with HBV.

In one embodiment, the method of the invention comprises a diagnostic assay for diagnosing HBV infection in a subject in need thereof. In one non-limiting example, an HBV-permissive DHH is contacted with a biological sample obtained from the subject and cultured as described herein. To make a diagnosis of HBV infection, the presence or absence or the level of the HBV titer, an HBV protein, an HBV nucleic acid, or a combination thereof, is assessed and compared with the level of at least one comparator control, such as a positive control, a negative control, a historical control, or a historical norm. The presence or absence or the level of the HBV titer, an HBV protein, an HBV nucleic acid, or a combination thereof, is assessed 1 day, 2 days, 3 days, 4 days, 5 days, 6 days, 7 days, 8 days, 9 days, 10 days, or more days after the DHH was contacted with the biological sample obtained from the subject. The presence or absence or the level of the HBV titer, an HBV protein, an HBV nucleic acid, or a combination thereof, can be assessed in cell culture media, in cells, or in combinations there. In one embodiment, the biological sample is serum. In another embodiment, the biological sample is HBVser.

In a further embodiment, the method of the invention comprises an assay for monitoring the effectiveness of an HBV treatment administered to a subject in need thereof. The method includes determining whether the level of HBV in a biological sample obtained from the subject is modulated upon administration of the treatment. The assay can be performed before, during or after a treatment has been administered, or any combination thereof. In this method, an HBV-permissive DHH is contacted with a biological sample obtained from the subject and cultured as elsewhere described herein. The presence or absence or level of the HBV titer, an HBV protein, an HBV nucleic acid, or a combination thereof, is assessed and compared with the level of at least one comparator control, such as a pre-treatment sample, a prior post-treatment sample, a positive control, a negative control, a historical control, or a historical norm. The presence or absence or the level of the HBV titer, an HBV protein, an HBV nucleic acid, or a combination thereof, is assessed 1 day, 2 days, 3 days, 4 days, 5 days, 6 days, 7 days, 8 days, 9 days, 10 days, or more days after the DHH was contacted with the biological sample obtained from the subject. The presence or absence or the level of the HBV titer, an HBV protein, an HBV nucleic acid, or a combination thereof, can be assessed in cell culture media, in cells, or in combinations there. In one embodiment, the biological sample is serum. In another embodiment, the biological sample is HBVser. Comparing the presence or absence or the level of the HBV titer, an HBV protein, an HBV nucleic acid, or a combination thereof, before treatment and after treatment, indicates whether the administered treatment is modulating the infection. When the level of the HBV titer, an HBV protein, an HBV nucleic acid, or a combination thereof, in the biological sample is decreased after administration of the treatment, the treatment is having a therapeutic effect on the infection.

In various embodiments, the level of HBV is assessed by measuring at least a fragment of an HBV polypeptide or an HBV nucleic acid. The term, “fragment,” as used herein, indicates that the portion of the polypeptide, mRNA or cDNA is of a length that is sufficient to identify the fragment as a fragment of an HBV polypeptide or an HBV nucleic acid.

The biological sample obtained from the subject can be a sample from any source which potentially contains virus, such as a body fluid or a tissue, or a combination thereof. A biological sample can be obtained by appropriate methods, such as, by way of examples, blood draw, fluid draw, or biopsy. A biological sample can be used directly, or can be processed, and the processed biological sample can then be used as the test sample.

The culture media assessed for the level of HBV titer, HBV polypeptide, or HBV nucleic acid can be assessed directly, or can be processed to enhance access to the HBV polypeptide or HBV nucleic acid. Alternatively or in addition, if desired, an amplification method can be used to amplify nucleic acids comprising all or a fragment of a nucleic acid in a test sample.

In various embodiments of the invention, methods of measuring an HBV polypeptide level include, but are not limited to, an immunochromatography assay, an immunodot assay, a Luminex assay, an ELISA assay, an ELISPOT assay, a protein microarray assay, a ligand-receptor binding assay, displacement of a ligand from a receptor assay, an immunostaining assay, a Western blot assay, a mass spectrophotometry assay, a radioimmunoassay (RIA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, a FACS assay, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

In some embodiments, quantitative hybridization methods, such as Southern analysis, Northern analysis, or in situ hybridizations, can be used (see Current Protocols in Molecular Biology, Ausubel, F. et al., eds., John Wiley & Sons, including all supplements). A “nucleic acid probe,” as used herein, can be a DNA probe or an RNA probe. The probe can be, for example, a gene, a gene fragment (e.g., one or more exons), a vector comprising the gene, a probe or primer, etc. For representative examples of use of nucleic acid probes, see, for example, U.S. Pat. Nos. 5,288,611 and 4,851,330. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate target nucleic acid. The hybridization sample is maintained under conditions which are sufficient to allow specific hybridization of the nucleic acid probe to the nucleic acid target. Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe having a target nucleic acid in the test sample, the level of the target nucleic acid in the sample can be assessed. More than one nucleic acid probe can also be used concurrently in this method. Specific hybridization of any one of the nucleic acid probes is indicative of the presence of the target nucleic acid, as described herein.

Alternatively, a peptide nucleic acid (PNA) probe can be used instead of a nucleic acid probe in the quantitative hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a target nucleic acid sequence. Hybridization of the PNA probe to a nucleic acid sequence is used to determine the level of the target nucleic acid in the sample.

In another embodiment, arrays of oligonucleotide probes that are complementary to target nucleic acid sequences in the biological sample obtained from a subject can be used to determine the level of nucleic acid in the sample. The array of oligonucleotide probes can be used to determine the level of the target nucleic acid alone, or the level of the target nucleic acid in relation to the level of one or more other nucleic acids in the sample. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a nucleic acid of interest is hybridized with the array and its level is quantified. Hybridization and quantification are generally carried out by methods described herein and also in, e.g., published PCT Application Nos. WO 92/10092 and WO 95/11995, and U.S. Pat. No. 5,424,186. In brief, a target nucleic acid sequence is amplified by well-known amplification techniques, e.g., PCR. Typically, this involves the use of primer sequences that are complementary to the target nucleic acid. Asymmetric PCR techniques may also be used. Amplified target, generally incorporating a label, is then hybridized with the array under appropriate conditions. Upon completion of hybridization and washing of the array, the array is scanned to determine the quantity of hybridized nucleic acid. The hybridization data obtained from the scan is typically in the form of fluorescence intensities as a function of quantity, or relative quantity, of the target nucleic acid in the biological sample. The target nucleic acid can be hybridized to the array in combination with one or more comparator controls (e.g., positive control, negative control, quantity control, etc.) to improve quantification of the target nucleic acid in the sample.

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention includes carried out with radioactive elements or with nonradioactive molecules. Among the radioactive isotopes used, mention may be made of 32P, 33P, 35S or 3H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents.

Nucleic acids can be obtained from culture media or from cells using known techniques. Nucleic acid herein refers to RNA, including mRNA, and DNA, including cDNA. The nucleic acid can be double-stranded or single-stranded (i.e., a sense or an antisense single strand) and can be complementary to a nucleic acid encoding a polypeptide. The nucleic acid content may also be an RNA or DNA extraction performed on a sample, including a culture media sample, biological fluid and fresh or fixed tissue sample.

There are many methods known in the art for the detection and quantification of specific nucleic acid sequences and new methods are continually reported. A great majority of the known specific nucleic acid detection and quantification methods utilize nucleic acid probes in specific hybridization reactions. Preferably, the detection of hybridization to the duplex form is a Southern blot technique. In the Southern blot technique, a nucleic acid sample is separated in an agarose gel based on size (molecular weight) and affixed to a membrane, denatured, and exposed to (admixed with) the labeled nucleic acid probe under hybridizing conditions. If the labeled nucleic acid probe forms a hybrid with the nucleic acid on the blot, the label is bound to the membrane.

In the Southern blot, the nucleic acid probe is preferably labeled with a tag. That tag can be a radioactive isotope, a fluorescent dye or the other well-known materials. Another type of process for the specific detection of nucleic acids in a biological sample known in the art are the hybridization methods as exemplified by U.S. Pat. No. 6,159,693 and No. 6,270,974, and related patents. To briefly summarize one of those methods, a nucleic acid probe of at least 10 nucleotides, preferably at least 15 nucleotides, more preferably at least 25 nucleotides, having a sequence complementary to a nucleic acid of interest is hybridized in a sample, subjected to depolymerizing conditions, and the sample is treated with an ATP/luciferase system, which will luminesce if the nucleic sequence is present. In quantitative Southern blotting, the level of the nucleic acid of interest can be compared with the level of a second nucleic acid of interest, and/or to one or more comparator control nucleic acids (e.g., positive control, negative control, quantity control, etc.).

Many methods useful for the detection and quantification of nucleic acid takes advantage of the polymerase chain reaction (PCR). The PCR process is well known in the art (U.S. Pat. No. 4,683,195, No. 4,683,202, and No. 4,800,159). To briefly summarize PCR, nucleic acid primers, complementary to opposite strands of a nucleic acid amplification target sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as discussed elsewhere herein. Most preferably the detection of the duplex is done using at least one primer directed to the nucleic acid of interest. In yet another embodiment of PCR, the detection of the hybridized duplex comprises electrophoretic gel separation followed by dye-based visualization.

Typical hybridization and washing stringency conditions depend in part on the size (i.e., number of nucleotides in length) of the oligonucleotide probe, the base composition and monovalent and divalent cation concentrations (Ausubel et al., 1994, eds Current Protocols in Molecular Biology).

In a preferred embodiment, the process for determining the quantitative and qualitative profile of the nucleic acid of interest according to the present invention includes characterized in that the amplifications are real-time amplifications performed using a labeled probe, preferably a labeled hydrolysis-probe, capable of specifically hybridizing in stringent conditions with a segment of the nucleic acid of interest. The labeled probe is capable of emitting a detectable signal every time each amplification cycle occurs, allowing the signal obtained for each cycle to be measured.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market, and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present invention includes the Tm, which is in the range of about 65° C. to 75° C. Preferably, the Tm for any one of the hydrolysis-probes of the present invention includes in the range of about 67° C. to about 70° C. Most preferably, the Tm applied for any one of the hydrolysis-probes of the present invention is about 67° C.

In one aspect, the invention includes a primer that is complementary to a nucleic acid of interest, and more particularly the primer includes 12 or more contiguous nucleotides substantially complementary to the nucleic acid of interest. Preferably, a primer featured in the invention includes a nucleotide sequence sufficiently complementary to hybridize to a nucleic acid sequence of about 12 to 25 nucleotides. More preferably, the primer differs by no more than 1, 2, or 3 nucleotides from the target flanking nucleotide sequence In another aspect, the length of the primer can vary in length, preferably about 15 to 28 nucleotides in length (e.g., 16, 17, 18, 19, 20, 21, 22, 23, 24, 25, 26, or 27 nucleotides in length).

### Methods of Identifying a Modulator of HBV Infection

The current invention relates to a method of identifying a compound that modulates HBV infection. In some embodiments, the method of identifying of the invention identifies an HBV infection inhibitor compound that diminishes HBV infection in the HBV culture system of the invention. In other embodiments, the method of identifying of the invention identifies an HBV infection activator compound that increases HBV infection in the HBV culture system of the invention. In various embodiments, the level of HBV infection can be assessed by measuring the level of an HBV protein, or by measuring the level of an HBV nucleic acid, or a combination thereof. The invention further comprises compositions comprising the modulator of HBV infection, identified by the methods described herein.

In one embodiment, the invention comprises a method of identifying a test compound as a modulator of HBV infection. Generally, the method of identifying a test compound as a modulator of HBV infection includes comparing a parameter of HBV infection in the presence of a test compound with a parameter of HBV infection in the absence of the test compound. Thus, in some embodiments, the method includes the steps of: placing at least one HBV-permissive DHH in culture medium in a first container, contacting the at least one HBV-permissive DHH in the first container with an HBV in the absence of the test compound, determining the level of HBV in the culture medium in the first container in the absence of the test compound; and placing at least one HBV-permissive DHH in culture medium in a second container, contacting the at least one HBV-permissive DHH in the second container with an HBV in the presence of the test compound, determining the level of HBV in the culture medium in the second container in the presence of the test compound; and comparing the level of HBV in the presence of the test compound with the level of HBV in the absence of the test compound; and identifying the test compound as a modulator of HBV infection when the level of HBV in the presence of the test compound is different than level of HBV in the absence of the test compound. In one embodiment, when the level of HBV is higher in the presence of the test compound, the test compound is identified as an HBV infection activator. In another embodiment, when the level of HBV is lower in the presence of the test compound, the test compound is identified as an HBV infection inhibitor. In various embodiments, the level of HBV is determined by measuring the HBV titer, an HBV nucleic acid, an HBV polypeptide, and combinations thereof. Suitable test compounds include, but are not limited to, a chemical compound, a protein, a peptide, a peptidomemetic, an antibody, a nucleic acid, an antisense nucleic acid, an shRNA, a ribozyme, and a small molecule chemical compound.

In one embodiment, the HBV-permissive DHH is permissive for infection by HBVcc. In another embodiment, the HBV-permissive DHH is permissive for infection by HBVser. In a further embodiment, the HBV-permissive DHH is permissive for infection by both HBVcc and HBVser. In various embodiments, the HBV-permissive DHH is permissive for infection by at least one of the HBV genotypes selected from the group consisting of genotype A, B, C, D, E, F, G and H. In a particular embodiment, the HBV-permissive DHH is permissive for infection by HBV that is resistant to at least one drug.

Other methods, as well as variation of the methods disclosed herein will be apparent from the description of this invention. In various embodiments, the test compound concentration in the screening assay can be fixed or varied. A single test compound, or a plurality of test compounds, can be tested at one time. Suitable test compounds that may be used include, but are not limited to, proteins, nucleic acids, antisense nucleic acids, small molecules, antibodies and peptides.

The invention relates to a method for screening test compounds to identify a modulator compound by its ability to modulate the level of HBV infection in the HBV culture system of the invention, by measuring HBV infection parameters in the presence and absence of the test compound.

The test compounds can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam et al., 1997, Anticancer Drug Des. 12:45).

Examples of methods for the synthesis of molecular libraries can be found in the art, for example, in: DeWitt et al., 1993, Proc. Natl. Acad. USA 90:6909; Erb et al., 1994, Proc. Natl. Acad. Sci. USA 91:11422; Zuckermann et al., 1994, J. Med. Chem. 37:2678; Cho et al., 1993, Science 261:1303; Carrell et al., 1994, Angew. Chem. Int. Ed. Engl. 33:2059; Carell et al., 1994, Angew. Chem. Int. Ed. Engl. 33:2061; and Gallop et al., 1994, J. Med. Chem. 37:1233.

Libraries of compounds may be presented in solution (e.g., Houghten, 1992, Biotechniques 13:412-421), or on beads (Lam, 1991, Nature 354:82-84), chips (Fodor, 1993, Nature 364:555-556), bacteria (Ladner U.S. Pat. No. 5,223,409), spores (Ladner U.S. Pat. No. '409), plasmids (Cull et al., 1992, Proc. Natl. Acad. Sci. USA 89:1865-1869) or on phage (Scott and Smith, 1990, Science 249:386-390; Devlin, 1990, Science 249:404-406; Cwirla et al., 1990, Proc. Natl. Acad. Sci. USA 87:6378-6382; Felici, 1991, J. Mol. Biol. 222:301-310; and Ladner supra).

In situations where “high-throughput” modalities are preferred, it is typical that new chemical entities with useful properties are generated by identifying a chemical compound (called a “lead compound”) with some desirable property or activity, creating variants of the lead compound, and evaluating the property and activity of those variant compounds.

In one embodiment, high throughput screening methods involve providing a library containing a large number of test compounds potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

### Genetically-Modified HBV-Permissive and Non-Permissive Cells

The invention also includes genetically-modified HBV-permissive and non-permissive DHH. In one embodiment, the stem cell used to derive an HBV-permissive DHH, using the methods described elsewhere herein, is genetically modified. In another embodiment, the stem cell used to derive an HBV-non-permissive DHH, using the methods described elsewhere herein, is genetically modified.

In one embodiment, the HBV-non-permissive DHH is derived from a genetically-modified stem cell possessing a genetic modification rendering the stem cell, and its DHH progeny, resistant to infection by HBV. In various embodiments, the genetic modification reduces or eliminates a host cell component necessary to render the cell permissive to HBV infection. Non-limiting examples of host cell components that can be reduced or eliminated to render the cell non-permissive to HBV infection include receptors and co-receptors. In a one embodiment, the genetic modification results in the reduction or elimination of cyclophilin A in the DHH. In another embodiment, the genetic modification results in the reduction or elimination of PI4KIIIα in the DHH. In a particular embodiment, stem cells isolated from a subject are genetically-modified and used to derive HBV-non-permissive DHH that are transplanted into the same, or a different, subject, to populate the subject with a population of HBV-non-permissive DHH.

The stem cells may be genetically modified using any method known to the skilled artisan. See, for instance, Sambrook et al. (2001, Molecular Cloning: A Laboratory Manual, Cold Spring Harbor Laboratory Press, Cold Spring Harbor, N.Y.), and in Ausubel et al,. Eds, (1997, Current Protocols in Molecular Biology, John Wiley & Sons, New York, N.Y.). For example, a stem cell may be exposed to an expression vector comprising a nucleic acid including a transgene, such that the nucleic acid is introduced into the cell under conditions appropriate for the transgene to be expressed within the cell. The transgene generally is an expression cassette, including a polynucleotide operably linked to a suitable promoter. The polynucleotide can encode a protein, or it can encode biologically active RNA (e.g., antisense RNA, shRNA, or a ribozyme). Thus, for example, the polynucleotide can encode a gene conferring resistance HBV infection.

Within the expression cassette, the coding polynucleotide is operably linked to a suitable promoter. Examples of suitable promoters include prokaryotic promoters and viral promoters (e.g., retroviral LTRs, lentiviral LTRs, immediate early viral promoters (IEp), such as herpesvirus IEp (e.g., ICP4-IEp and ICP0-IEEp), cytomegalovirus (CMV) IEp, and other viral promoters, such as Rous Sarcoma Virus (RSV) promoters, and Murine Leukemia Virus (MLV) promoters). Other suitable promoters are eukaryotic promoters or enhancers (e.g., the rabbit (3-globin regulatory elements), constitutively active promoters (e.g., the (3-actin promoter, etc.), signal specific promoters (e.g., inducible promoters such as a promoter responsive to RU486, etc.), and tissue-specific promoters (e.g., liver-specific promoter). It is well within the skill of the art to select a promoter suitable for driving gene expression in a predefined cellular context. The expression cassette can include more than one coding polynucleotide, and it can include other elements (e.g., polyadenylation sequences, sequences encoding a membrane-insertion signal or a secretion leader, ribosome entry sequences, transcriptional regulatory elements (e.g., enhancers, silencers, etc.), and the like), as desired.

The expression cassette containing the transgene should be incorporated into a genetic vector suitable for delivering the transgene to the cells. Depending on the desired end application, any such vector can be so employed to genetically modify the cells (e.g., plasmids, naked DNA, viruses such as adenovirus, adeno-associated virus, herpesviruses, lentiviruses, papillomaviruses, retroviruses, etc.). Any method of constructing the desired expression cassette within such vectors can be employed, many of which are well known in the art (e.g., direct cloning, homologous recombination, etc.). The choice of vector will largely determine the method used to introduce the vector into the cells (e.g., by protoplast fusion, calcium-phosphate precipitation, gene gun, electroporation, DEAE dextran or lipid carrier mediated transfection, infection with viral vectors, etc.), which are generally known in the art.

Examples of techniques sufficient to direct persons of skill through in vitro amplification methods, including the polymerase chain reaction (PCR), the ligase chain reaction (LCR), and other DNA or RNA polymerase-mediated techniques are found in Sambrook et al., Molecular Cloning: A Laboratory Manual, volumes 1-3 (3rd ed., Cold Spring Harbor Press, NY 2001).

Once the nucleic acid for a protein is cloned, a skilled artisan may express the recombinant gene(s) in a variety of stem and liver cells. It is expected that those of skill in the art are knowledgeable in the numerous expression systems available for expressing the desired transgene.

### HBV Kits

The present invention also pertains to kits useful in the methods of the invention. Such kits comprise various combinations of components useful in any of the methods described elsewhere herein, including for example, hybridization probes or primers (e.g., labeled probes or primers), antibodies, reagents for detection of labeled molecules, materials for the amplification of nucleic acids, a stem cell, components for deriving an HBV-permissive DHH from a stem cell, an HBV-permissive DHH cell, materials for quantitatively analyzing an HBV polypeptide and/or an HBV nucleic acid, and instructional material. For example, in one embodiment, the kit comprises components useful for deriving an HBV-permissive DHH from a stem cell.

In one embodiment, the kit comprises the components of a diagnostic assay for diagnosing HBV infection in a subject in need thereof, containing instructional material and the components for determining the level of HBV in a biological sample obtained from the subject, the genotype of HBV in a biological sample obtained from a subject, and/or the drug resistance status of HBV in a biological sample obtained from a subject. In various embodiments, determining the level, genotype or drug resistance status of HBV in a biological sample obtained from the subject requires a comparison to at least one comparator control contained in the kit, such as a positive control, a negative control, a historical control, a historical norm, or the level of another reference molecule in the biological sample.

In a further embodiment, the kit comprises the components of an assay for monitoring the effectiveness of a treatment administered to a subject in need thereof, containing instructional material and the components for determining the level of HBV in a biological sample obtained from the subject, the genotype of HBV in a biological sample obtained from a subject, and/or the drug resistance status of HBV in a biological sample obtained from a subject. In various embodiments, determining the level, genotype or drug resistance status of HBV in a biological sample obtained from the subject requires a comparison to at least one comparator control contained in the kit, such as a positive control, a negative control, a historical control, a historical norm, or the level of another reference molecule in the biological sample.

### Other HV

The skilled artisan will understand that each of the compositions, methods and kits of the invention described herein as comprising HCV or HBV, can, in the alternative, comprise another HV, including HAV, HDV or HEV.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Productive Infection of Stem Cell-Derived Human Hepatocytes by Hepatitis C Virus Reveal Host Determinants of Viral Permissiveness

The data described herein demonstrate that hepatic cells derived by directed differentiation of stem cells, including iPSCs, can support HCV infection. Complete life cycles of HCV infection were completed starting with HCV entry and ending with secretion of infectious viral particles into culture media. Infection of DHHs was sensitive to replication inhibitors as well as entry blockers. Four different variants of JFH-1, including a J6/JFH hybrid (GLuc), were used to produce HCVcc used in the studies described herein. Both wt sequence (JFH-FLAG) and adaptive mutants (SAV and Mut4-6) were able to replicate in DHHs, indicating that the ability for DHHs to support HCV infection was not dependent on particular isoforms or mutations. In addition, infection with a genotype 1b clinical isolate was also achieved, demonstrating the feasibility of using DHHs to study this particular genotype that, though prevalent in patients, so far has largely resisted in vitro infection studies. Beyond the utility of being able to culture a variety of HCV genotypes, the direct infection of DHH using patient serum has broad significance for challenging research areas such as the characterization of drug resistance mechanisms and functional characterization of authentic HCV particles.

The DHH systems described herein represent an important finding in the field of in vitro model systems for HCV infection. In contrast to cell lines derived from tumor tissues, DHHs are non-cancerous and retain important functions of primary hepatocytes such as secretion of ALB, glycogen storage, LDL uptake, and cytochrome P450 function, as described herein. DHHs also offer advantages over PHHs as being more accessible, genetically malleable, and unlimited in supply.

As described herein, genetic modification of pluripotent stem cells prior to directed differentiation is an attractive approach to obtain specific cell types with a desired phenotype (e.g., resistance to HCV infection). In the context of HCV infection and liver disease, stem cell lines with essential cellular cofactors knocked out or knocked down can serve as a renewable source to produce HCV-resistant hepatocyte-like cells in vitro, which can in turn be used in transplant procedures and experiments.

RNA interference (RNAi) appears to function efficiently in all cell types and represents an alternative to gene knock-out approaches, especially when partial suppression of a cofactor is sufficient to reduce viral infection in a meaningful way. In studies described herein, lentiviral vector-mediated expression of shRNA is maintained in long-term differentiation cultures and CyPA knockdown in both hESCs and DHHs has no apparent adverse effects on either pluripotency or differentiation. As described herein, the CyPA knockdown DHHs were permissive to infection by a HCV mutant with reduced dependence, further indicating these modified cells retained hepatic features that encompass HCV's liver tropism.

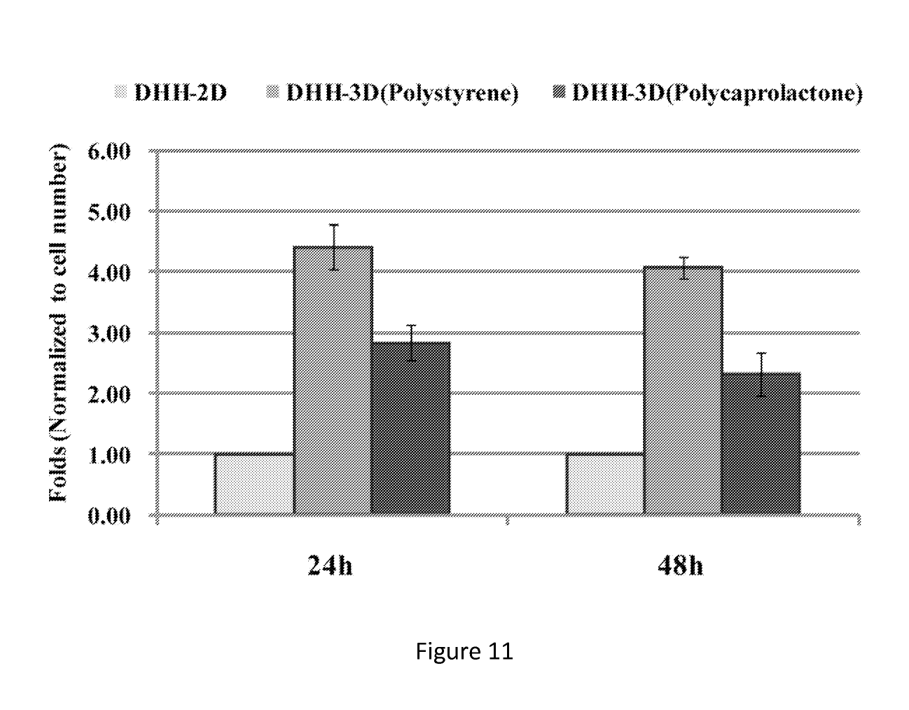

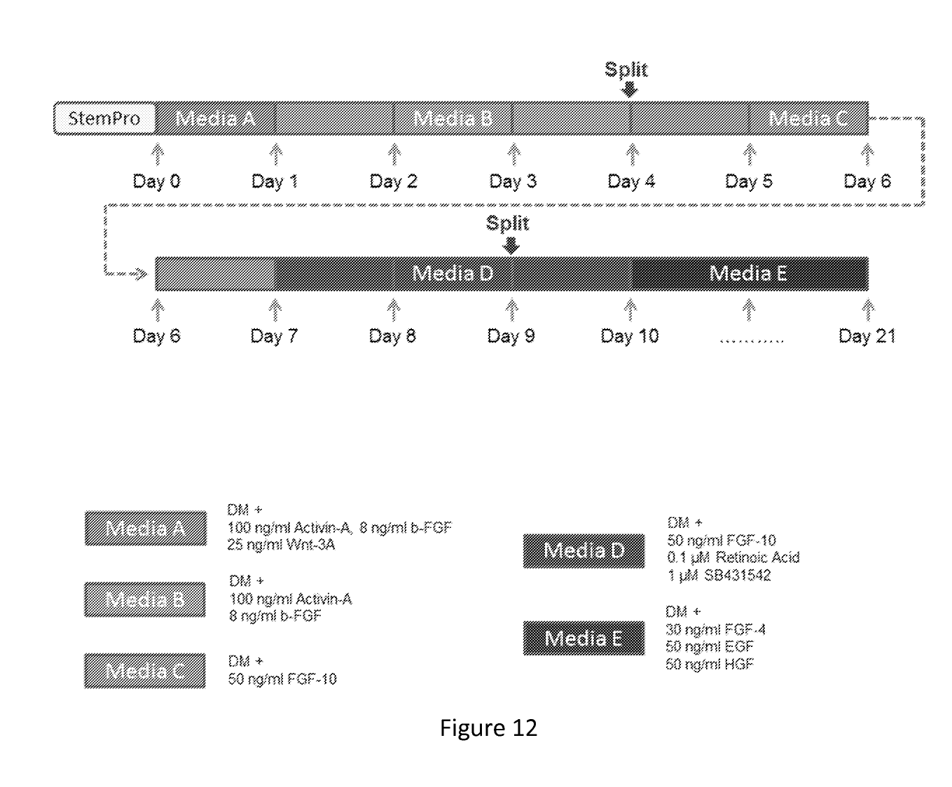

Preliminary experiments showed that DHH cultured in three-dimensional cell culture scaffolds (3-D Biotek, NJ) conferred higher infectivity to HCVcc (FIG. 11), suggesting the potential of improving DHH infection efficiency using tissue engineering methodology. Interestingly, as the data described herein demonstrate, the relative efficiencies for Huh-7.5 and DHHs to support infection by HCVcc versus that by HCVser were distinctly different. HCVcc infected DHHs less efficiently than Huh-7.5 cells, while HCVser specifically infected DHHs but not Huh-7.5, suggesting that DHHs represent a more physiologically relevant model for infection by clinical isolates of HCV.

Viral tropism for a specific cell type is typically associated with the expression of tissue-specific cofactors (e.g. receptors). As described herein, the induction of miR-122 expression correlated with hepatic specification and preceded the transition to HCV susceptibility, confirming the connection between this liver-specific microRNA and host-restriction to HCV infection in non-hepatic cells, as first reported by Joplin et al. (2005, Science. 309:1577-1581). Occurring with FGF-10 treatment, and in combination with the withdrawal of Activin A, miR-122 expression was induced by more than several hundred-fold. Consistent with the findings described herein, the connection between FGF-10 and miR-122 induction can be the hepatocyte nuclear factor 4 alpha (HNF4α), which has been reported to bind the miR-122 promoter and activate pri-miR-122 transcription (2011, Li et al., J Hepatol. 55:602-611).

In addition to miR-122, EGFR and EphA2, two RTKs that contribute to HCV entry process through their kinase function, were specifically regulated in the permissive cells described herein. Of note, the maturation medium that contains EGF, although not necessary to confer permissiveness, enhanced HCVcc infection of day-10 cells. The expression of both ephrin A1 (EFNA1), which is the ligand for EphA2, and EFNB2 also increased from day 7 to day 10. EFNB2 is known to be the membrane-bound ligand for the EphB and serves as a cellular receptor for Nipah virus, thus, EFNB2 may too play role in the HCV entry process.

The studies disclosed herein describe for the first time, a cell rendered permissive to HCV infection and replication by the treatment of defined chemical compounds. These important finding provide a multitude of opportunities to study HCV, HCV infection, and ways to interfere with HCV infection, including the identification and characterization of receptors, co-receptors, cellular components and signaling pathways involved in HCV

Also described herein is the genetic modification of pluripotent stem cells prior to differentiation to generate viral-resistant hepatocytes. In addition to the direct application in studies of cellular components involved in HCV infection, or other diseases with a genetic component, the concept illustrated in this study can be coupled with patient-specific iPSCs technology, to generate a multitude of cell types with desired phenotypes for cell therapy.

The materials and methods employed in these experiments are now described.

### Growth Factors, Chemicals and Antibodies

Basic FGF (b-FGF), Stem Pro hESC SFM, β-mercaptoethanol, and Geltrex were purchased from Invitrogen (Carlsbad, Calif.). FGF-10, FGF-4, EGF, and HGF from PeproTech (Rocky Hill, N.J.); SB 431542 and retinoic acid (RA) from Sigma Aldrich (St Luis, Mo.); Wnt-3A from Stemgent (San Diego, Calif.); Accutase from Innovative Cell Technologies (San Diego, Calif.); activin-A from R&D systems (Minneapolis, Minn.); and Probumin from Millipore (Billerica, Mass.).

TABLE 1
Antibodies used in the experiments described herein.

| Antigen | Provider | Catalog Number |
|---|---|---|
| HCV Core, NS3, | BioFront Technologies | 3D11/4F5/2H1/4F9, 2E3, |
| and NS5A | | 7B5 |
| Human Oct-4 | Santa Cruz Biotechnology | sc-5279 |
| Human CXCR4 | NIH AIDS Regents | MAB172 |
| | Program | |
| ALB, FLAG | Sigma Aldrich | SAB3300097, F7525 |
| SR-BI | Novus Biologicals | NB400-101 |
| Claudin-1, | Invitrogen | 374900, 18-0234 |
| Cytokeratin-7 | | |
| CD81 | BD Pharmingen | 555675 |
| Occludin | Abcam | ab31721 |
| Human | DAKO | ABIN370517 |
| α-fetoprotein | | |
| Human DDX-3 | Dr. Robin Reed (Harvard | n.a |
| | Medical School). | |
| EGFR, EphA2 | Thermo Scientific | MA5-15284, PA1-1110 |
| PI4KIIIα | Cell Signaling Technology | 4902 |
| Cyclophilin A | Biomol Enzo Life Sciences | BML-SA296-0100 |

hESC, iPSC, and Primary Human Hepatocytes

Human ESC line H9 and iPS line iPS.K3 cells were obtained from WiCell Research Institute and Stephen Duncan at Medical College of Wisconsin, respectively. Stem cells were maintained on Geltrex coated culture plates, in Stem Pro media (Invitrogen, Carlsbad, Calif.). Freshly isolated PHHs were purchased from Celsis In Vitro Technologies (Baltimore, Md.) and maintained according to provider's instructions. Lentiviral transduction to generate stable cells harboring shRNAs was performed as described (2008, Yang et al., J Virol. 82:5269-5278). All recombinant DNA procedures were performed according to NIH guidelines.

Differentiation of hESCs and iPSCs into Hepatic Cells

The base defined medium (DM) consisted of DMEM/F12 containing 10% Probumin, 0.2% β-Mercaptoethanol, 1% L-Alanyl-L-glutamine and 2% hESC supplement. Confluent cells were harvested using Accutase and then plated into culture dishes (Costar; Corning Life Sciences) precoated with Geltrex (1:200 dilution in DMEM/F-12) in StemPro media at a confluence level of 30-40%. The next day, media was changed to differentiation media A (DM+100 ng/ml activin-A+8 ng/ml b-FGF+25 ng/ml Wnt-3A) for 24 hours, followed by three days in differentiation media B (DM+100 ng/ml activin-A+8 ng/ml b-FGF). To induce hepatic differentiation, cells were then cultured in the presence of differentiation media C (DM+50 ng/ml FGF-10) for three days and then in the presence of differentiation media D (DM+50 ng/ml FGF-10+0.1 μM RA+1 μM SB431542) for three more days. The immature hepatocytes were then split 1:2 and grown in the differentiation media E (DM+30 ng/ml FGF-4+50 ng/ml EGF+50 ng/ml HGF) for 10 days with media changes of fresh media E every two or three days.

### HCVcc and HCVser Used in the Infections

All JFH-1 based HCVcc (Mut4-6, SAV, Jc1/GLuc2A, and DEYN-Jc1/GLuc2A) were produced in Huh-7.5 cells as previously described (2005, Lindenbach, et al., Science 309:623-626). Genotype 1b HCVser was obtained from a commercial supplier (Teragenix, Ft. Lauderdale, Fla.). All infections were done by incubating virus inoculum with cells for 6-9 hours before the cells were washed and changed into the appropriate medium for the specific cell type and differentiation stage. For the time course of DHHs permissiveness, infection of each time point was allowed to proceed for exactly 48 hours before cell harvesting and western blotting. Immunofluorescence analysis of HCV receptors and intracellular antigens. Cells were fixed in 4% paraformaldehyde in phosphate-buffered saline (PBS) at room temperature for 10 minutes and blocked with PBGB (PBS containing 10% normal goat serum, and 1% bovine serum albumin (BSA)) at room temperature for 2 hours. Cells were incubated with primary antibodies (anti-CD81, anti-SR-B1, anti-claudin 1 and anti-occludin, diluted in PBG at 1:200) at 4° C. overnight or 2 hours at room temperature. Isotype mouse or rabbit IgGs were used as negative controls. After four washes with PBSB (PBS with 0.1% BSA), FITC or TRITC-conjugated secondary antibody diluted at 1:500 was added and incubated at room temperature for 1 hour. Before being mounted with VECTASHIELD (H-1200, Vector Labs), cells were washed with PBSB three times and once with PBTG (PBS containing 0.1% Triton X-100, 10% normal goat serum, and 1% BSA). For intracellular staining, the cells were permeablized in PBST after fixing to allow access by primary antibody.

### HCV-Dependent Fluorescence Relocalization Assay

Lentiviral vectors expressing EGFP-IPS (TRIP-EGFP) or RFP-NLS-IPS (TRIP-NLS-RFP) were provided by Charles Rice and produced in 293-FT cells as described (2010, Jones et al., Nat Biotechnol. 28:167-171). Day-10 DHHs or Huh-7.5 cells, seeded on coverslips the day before, were transduced with the vectors for 24 hours before being infected by HCVcc or HCVser for 6 hours. The cells were cultured for 2-3 more days before the slides were fixed for fluorescence microscopy analysis.

### Microarray and RT-PCT Analysis

Complimentary DNA used for microarray hybridization was prepared as the following. Total RNAs from day-7 and day-10 cells were isolated using Qiagen RNeasy Mini kit and then converted the RNA into single-stranded cDNA with the Applied Biosystems High Capacity cDNA Reverse Transcription kit. The RNA/cDNA hybrids were denatured at 95° C. for 1 min and then treated with RNase A for 30 min at 37° C. The resulting cDNA was cleaned up with Qiagen PCR purification kit (catalog #28106) before used for fluorescent labeling. A Nimblegen 4 x 72K Expression Array was used for hybridization according to the manufacturer's instructions. Expression data and gene ontology analysis were done with ArrayStar (DNASTAR) and GOrilla. For RT-PCR, total RNA was isolated using TRIzol and then converted to first-strand cDNA using SuperScript III (Invitrogen) with oligo-dT serving as the RT primer. Resulting products served as templates for PCR analysis of HCV cofactors and receptors.

Real-Time RT-PCR Detection of Micro-RNA 122 (miR122)

To quantify miR-122 levels, TRIzol extracted RNA samples were reverse transcribed using TaqMan MicroRNA Reverse Transcription kit (Applied Biosystems) and the resulting cDNA served as templates for Real-Time PCR analysis using TaqMan MicroRNA Assay for miR-122 (Applied Biosystems). Albumin and HCV Core ELISA. Albumin ELISA was performed with a human Albumin ELISA kit (Bethyl Laboratories, Montgomery, Tex.) and HCV core ELISA with the HCV Antigen ELISA kit (Ortho-Clinical Diagnostics, Japan), according to manufacturer's instructions.

### Lentivirus-Mediated RNA Interference

Lentiviral vectors containing a shRNA directed at human CyPA has been described previously (2008, Yang et al., J Virol. 82:5269-5278). A shRNA directed at PI4KIIIα was constructed in a similar fashion. The shRNA target sequence of the PI4KIIIα mRNA is 5′-AAG CTA AGC CTC GGT TAC AGA-3′ (SEQ ID NO:1). Introduction of these vectors into stem cells was done via standard lentiviral transduction procedure (2004, Waninger et al., J Virol. 78:12829-12837) and selection of stable cells harboring shRNA was done by culturing the cells in Stem Pro medium supplemented with 600 ng/ml of puromycin.

The results of the experiments are now described.

In Vitro Differentiated Hepatocytes Derived from Either hESCs or iPSCs are Permissive to HCV Infection

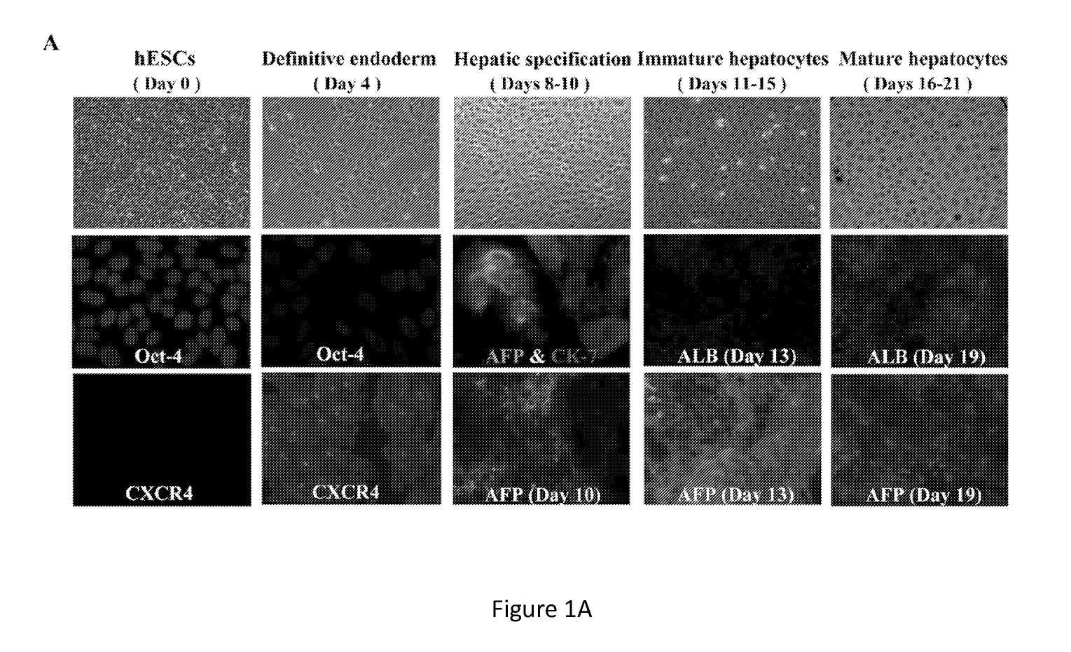

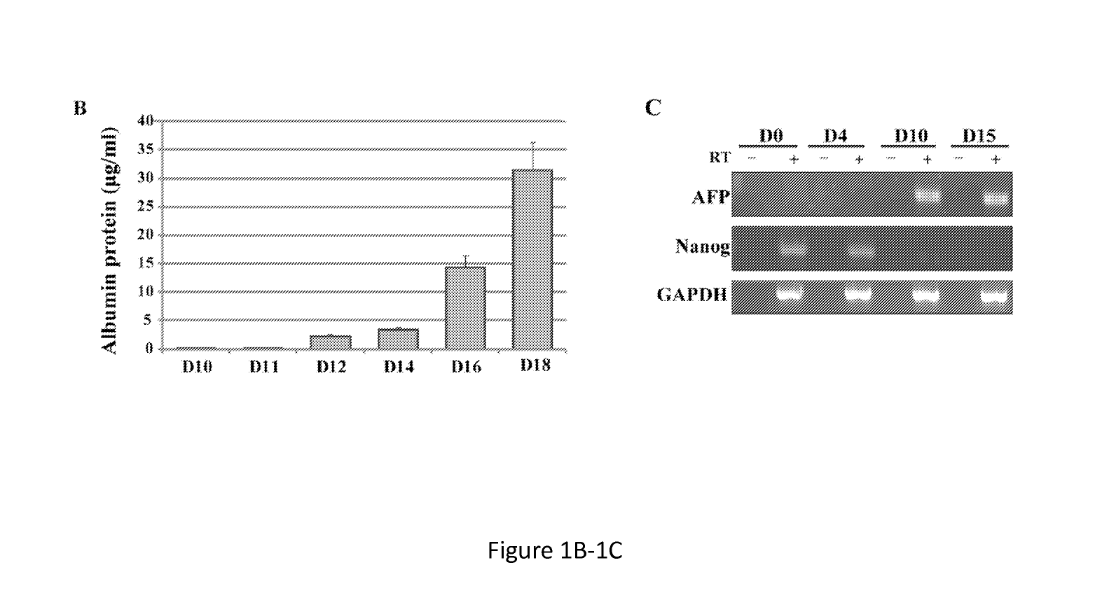

It was first determined whether DHHs derived from directed differentiation of hESCs or iPSCs were susceptible to infection by HCVcc. A serum-free protocol based on chemically defined culture media (2010, Touboul et al., Hepatology 51:1754-1765; 2007, McLean et al., Stem Cells 25:29-38) was used to differentiate the hESC line H9 (WA09) (1998, Thomson et al., Science. 282:1145-1147) or the iPSC line (iPS.K3) (2010, Si-Tayeb et al., Hepatology 51:297-305) into hepatic lineage cells that expressed various hepatic markers at different stages of differentiation (FIG. 1A). At day 10 post differentiation, the cells were positive for either alpha-fetoprotein (AFP) or cytokeratin-7 (CK-7) but not both, suggesting that they are of a composition similar to that of the bipotent hepatoblasts; AFP expression steadily increased in the next 5 days from 5% at day 10 to over 90% at day 16. The intensity of AFP stain then decreased when albumin (ALB) started to express towards the end of the differentiation protocol (FIG. 1A). Secretion of albumin into culture medium was evident starting from day 12 post-differentiation but was highest after 18 days post-differentiation (FIG. 1B). Reverse-transcriptase coupled PCR (RT-PCR) confirmed that AFP messenger RNA was induced at day 10 whereas the expression of the pluripotent marker Nanog extinguished (FIG. 1C).

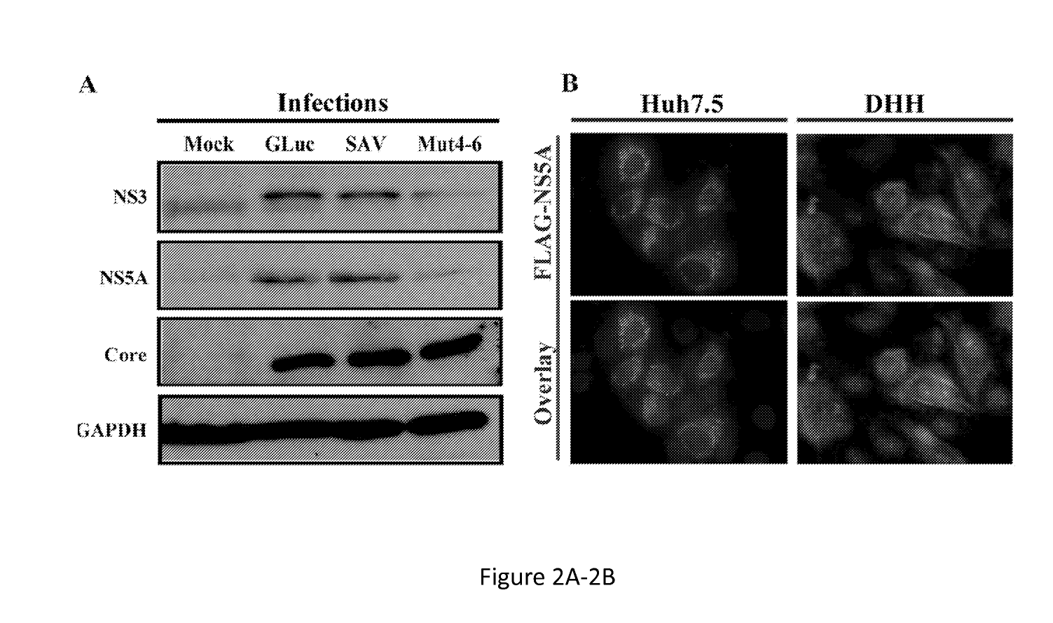

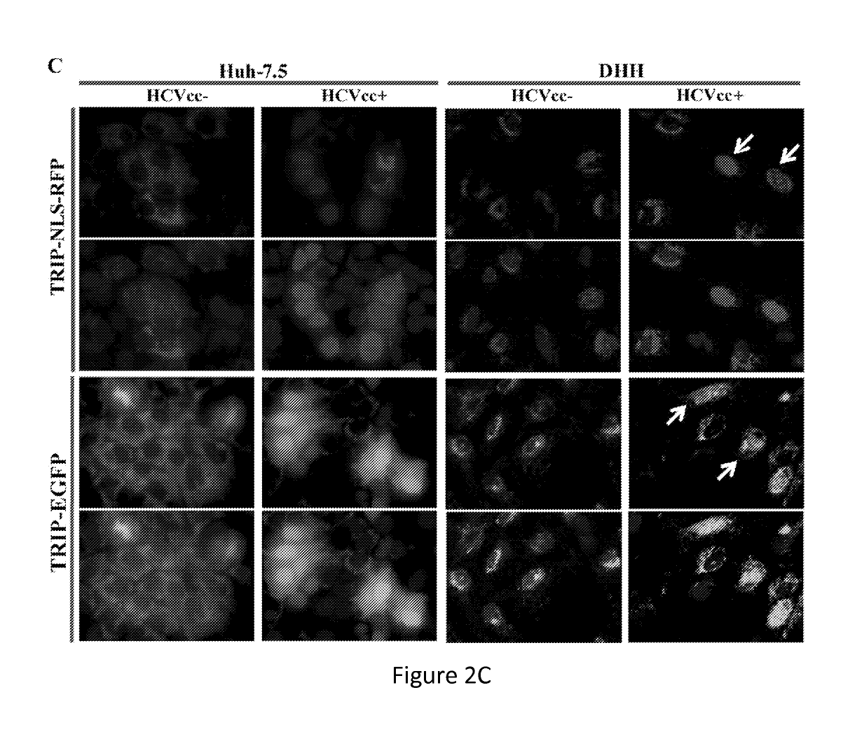

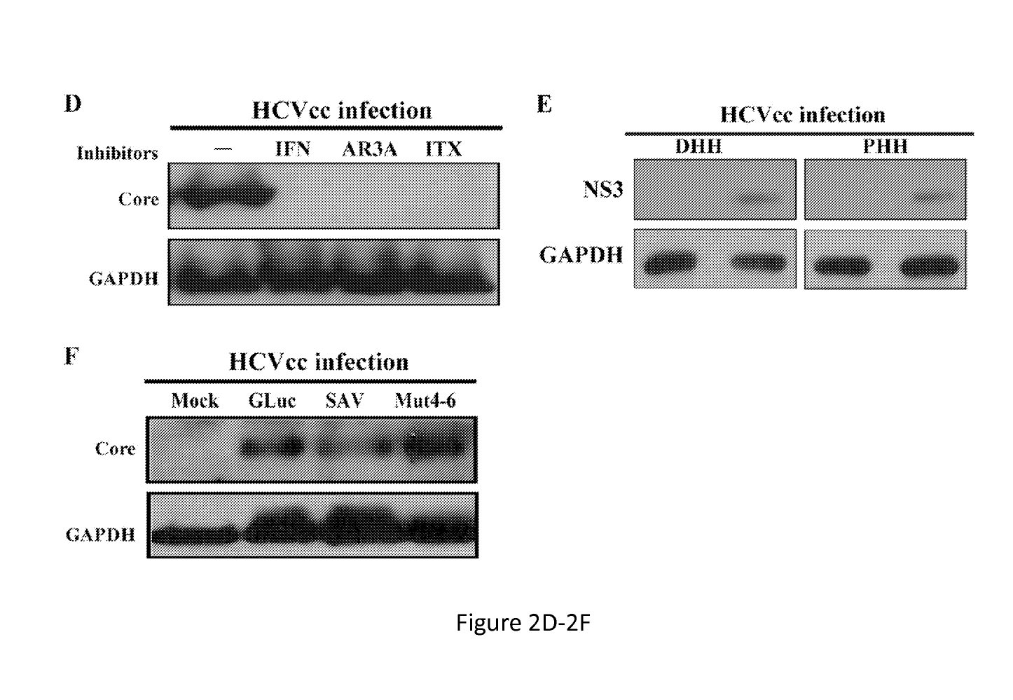

Three distinct variants of JFH-1 were used for the initial infection at day 13 and then collected cell lysates at the end of the differentiation period (day 21) for western blotting to detect HCV protein expression. Two of the JFH-1 genomes contained adaptive mutations that enhanced their infectious titers by at least 100 fold over the JFH-1 wt background. Mut4-6 has been reported previously (2007, Kaul et al., J Virol. 81:13168-13179) and the serially adapted virus (SAV) was obtained by repeated passage of JFH-1 HCVcc in Huh-7.5 cells. The third HCVcc variant is the J6/JFH chimera with a Gaussia luciferase (GLuc) reporter gene incorporated (2009, Phan et al., J Virol. 83:8379-8395). Expression of HCV proteins, core, NS3, and NS5A were readily detected by western blotting for all three HCVcc preparations (FIG. 2A). Intracellular expression of HCV antigen was also detectable by immunofluorescent staining following infection by a fourth JFH-1 variant that encoded a FLAG-tagged NS5A (FIG. 2B). In addition, introduction of a HCV-dependent fluorescence relocalization (HDFR) reporter construct (2010, Jones et al., Nat Biotechnol. 28:167-171) into the day-10 cells confirmed infection events in single cells by monitoring the nuclear translocation of a fluorescent protein upon cleavage of its mitochondria anchor by HCV NS3 (FIG. 2C). To determine if HCVcc infection of DHHs was dependent on viral glycoproteins and cell surface receptors, infection was performed in the presence of a neutralizing E2 antibody (2008, Law et al., Nat Med. 14:25-27) and a small molecule compound that inhibits the scavenger receptor class B type I (SR-BI) (2011, Syder et al., J Hepatol. 54: 48-55). Both efficiently blocked infection, as did the replication inhibitor IFN-α (FIG. 2D). A comparison of HCV expression levels in similarly infected DHHs (H9-derived) and PHHs (isolated from a patient) revealed that efficiency of infection in DHHs is comparable to that in PHHs (FIG. 2E). Finally, DHHs derived from an iPSC cell line (iPS.K3) also supported robust infection by all three derivatives of the JFH-1/HCVcc (FIG. 2F).

### DHHs Support Persistent Infection and Produce Infectious Particles

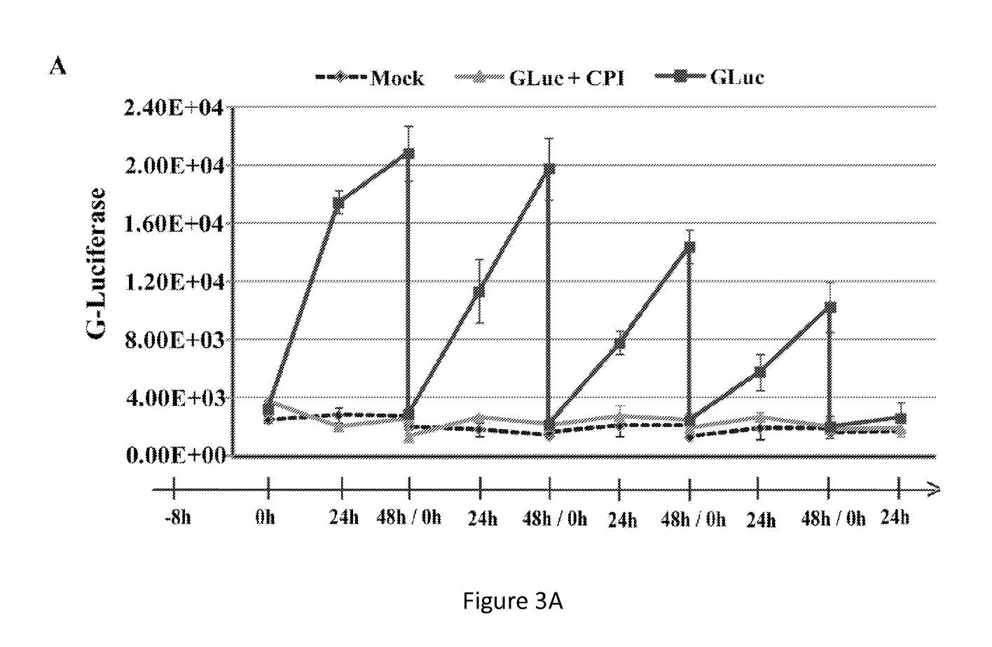

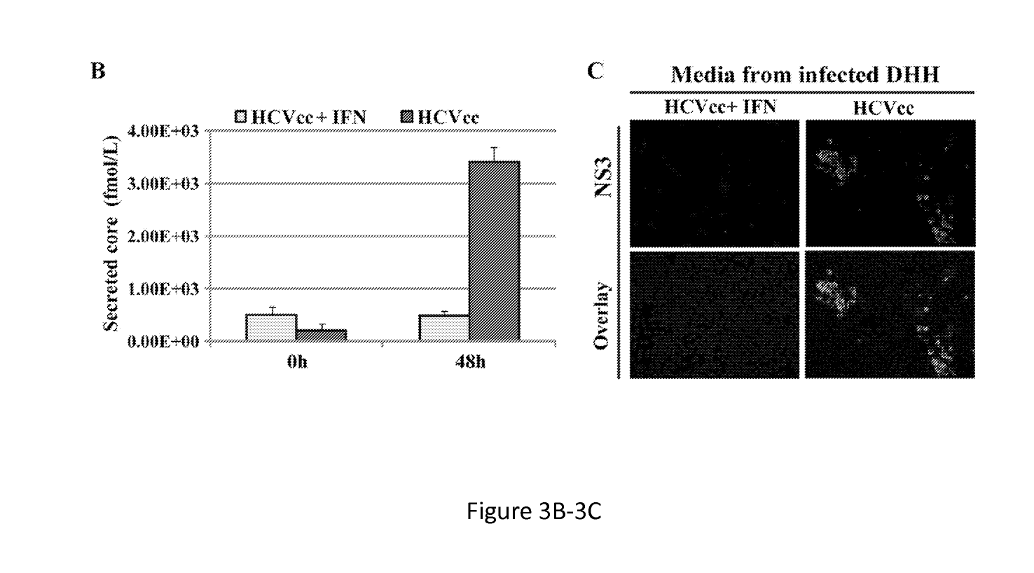

To verify continuous viral replication during the infection period, the secretion of Gaussia luciferase into the culture media by the DHHs infected with the GLuc reporter virus was monitored using a procedure previously used to monitor persistent HCV infection in microscale PHHs (2010, Floss et al., Proc Natl Acad Sci USA 107:3141-3145). After the initial infection, the viral inoculum was removed and replaced with fresh media, a fraction of which was then collected immediately (0 hour), 24 hours, and 48 hours after the virus removal. At the 48h time point, the cells were washed again and changed into fresh media which was then collected in a similar fashion. This process was repeated until day 21, when the DHHs became senescent and died off the plates. A gradual increase of the luciferase activity was detected in the culture media following each removal, whereas the signal increase was not observed in medium from either mock infected cells or from infected cells treated with cyclosporine A (CsA), an inhibitor of cyclophilins and HCV replication (2003, Watashi et al., Hepatology 38:1282-1288) (FIG. 3A). In addition to persistent replication, production of infectious viral particles was also achieved in DHHs infected with HCVcc. H9-derived DHHs was infected at day 11 post-differentiation and culture supernatants were collected 48 hours after infection. HCV core antigen was detected in the supernatant of the infected cells but not in that of the similarly infected but IFN-treated cells (FIG. 3B). To test if the core-positive culture supernatant contained infectious viral particles, these supernatants were used to reinfect Huh-7.5 cells. NS3-positive foci could be clearly detected in the reinfected cells (FIG. 3C), demonstrating that DHHs were capable of supporting infectious particle production.

Transition from Non-Permissive to Permissive Cells

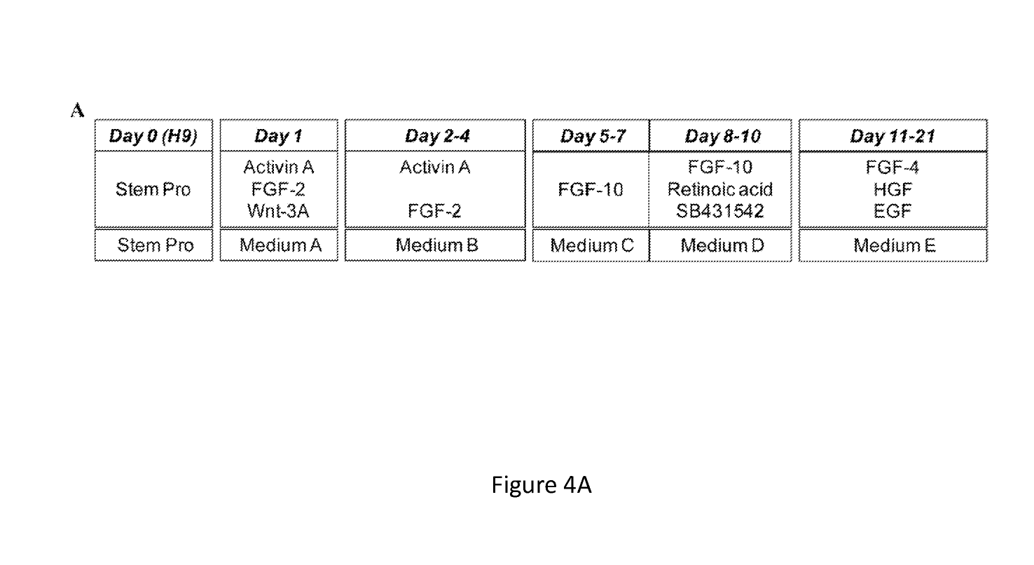

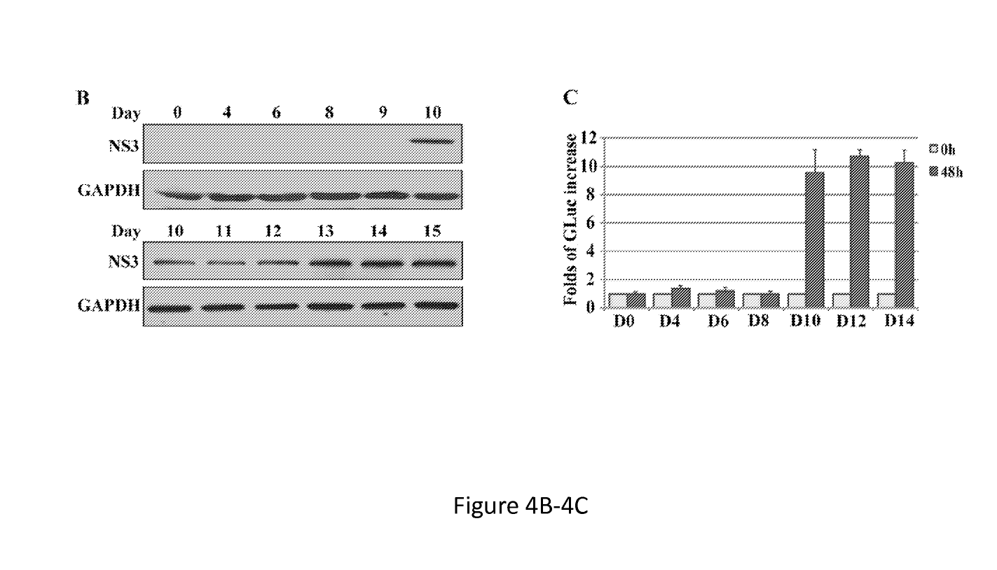

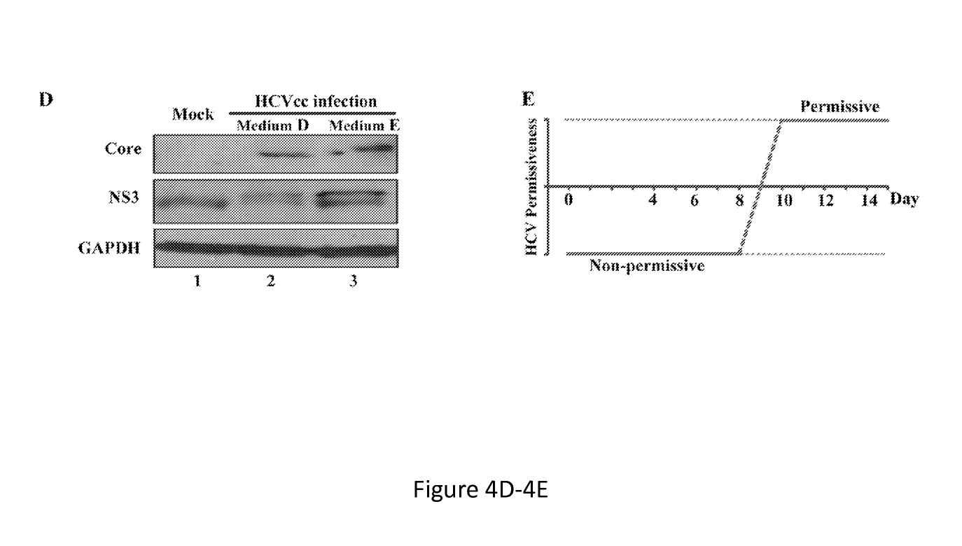

Next, the transition stage during differentiation that rendered the DHHs susceptible to HCV infection was determined. The hepatic differentiation protocol that was used involved five different medium compositions for the various stages of differentiation (FIG. 4A). A combination of Activin A, basic fibroblast growth factor (b-FGF), and Wnt-3A (Media A and B) was used to induce the differentiation of DE (days 1-4), which was cultured in a FGF-10-containing medium (Medium C) for three days (days 5-7) to initiate hepatic specification. After day 7, Medium C was supplemented with retinoic acid (RA) and a transforming growth factor-β (TGF-β) inhibitor, SB431542 and the cells were cultured for additional three days (days 8-10) in this medium (Medium D). Finally, the hepatocyte-like cells were allowed to mature in Medium E which contained hepatocyte growth factor (HGF), epidermal growth factor (EGF), and FGF-4 (days 11-21). The cells were infected at different time points with GLuc-based HCVcc for 6 hours, the inoculum was removed, and then infection was by measuring both intracellular NS3 expression and luciferase activity in the medium 48 hours post infection. A clear infection signal was detected in cells at day 10 and later post-differentiation, whereas the stem cells (H9), the DE, or cells up to day 9 post-differentiation could not be infected (FIGS. 4B and 4C). Because the day-10 cells normally were immediately changed into the Medium E following the removal of the viral input, it was evaluated whether the maturation media was required for the infection. To address this question, an experiment was performed where the infected day-10 cells were either kept in Medium D (FGF-10, RA, and SB) or changed into Medium E (HGF, EGF, and FGF-4). Both samples were collected at day 21 and subjected to immunoblotting for detection of HCV proteins. The maturation medium was not required for HCV-permissiveness as both cell populations became infected. However, the maturation process may further increase the infection efficiency (FIG. 4D, compare lanes 2 and 3). These results identify a discrete temporal switch during the hepatic differentiation process that marks the transition to permissiveness for HCV infection (FIG. 4E).

Cellular Changes Associated with HCV-Permissiveness

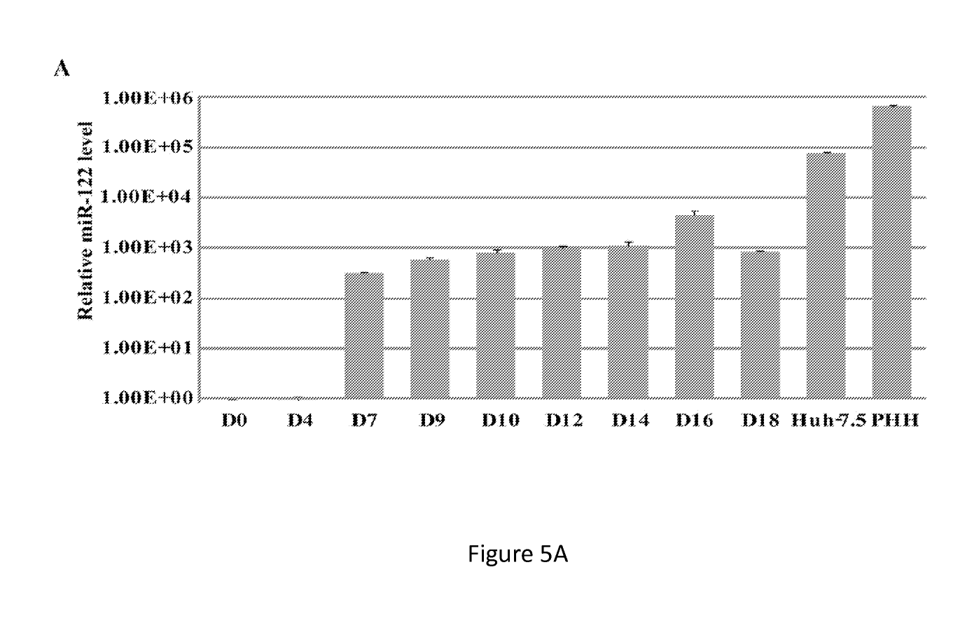

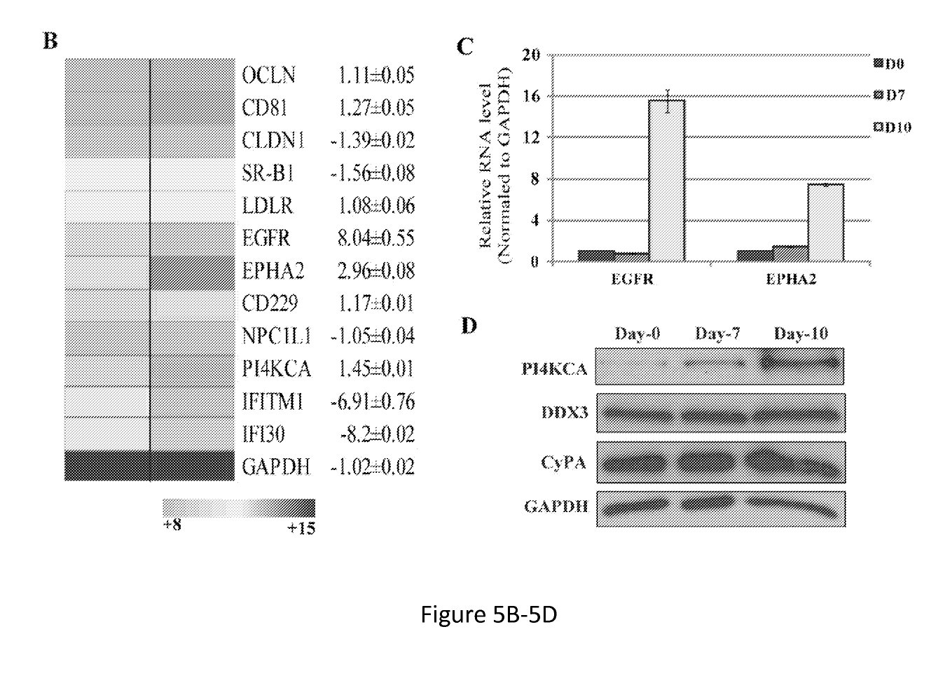

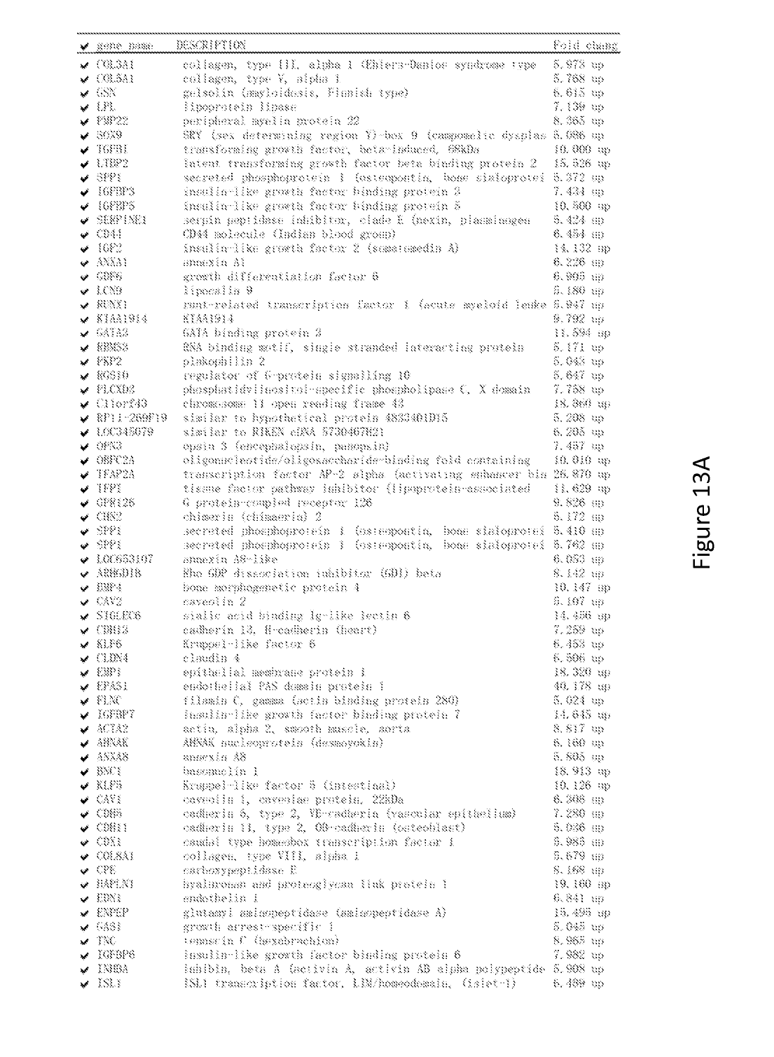

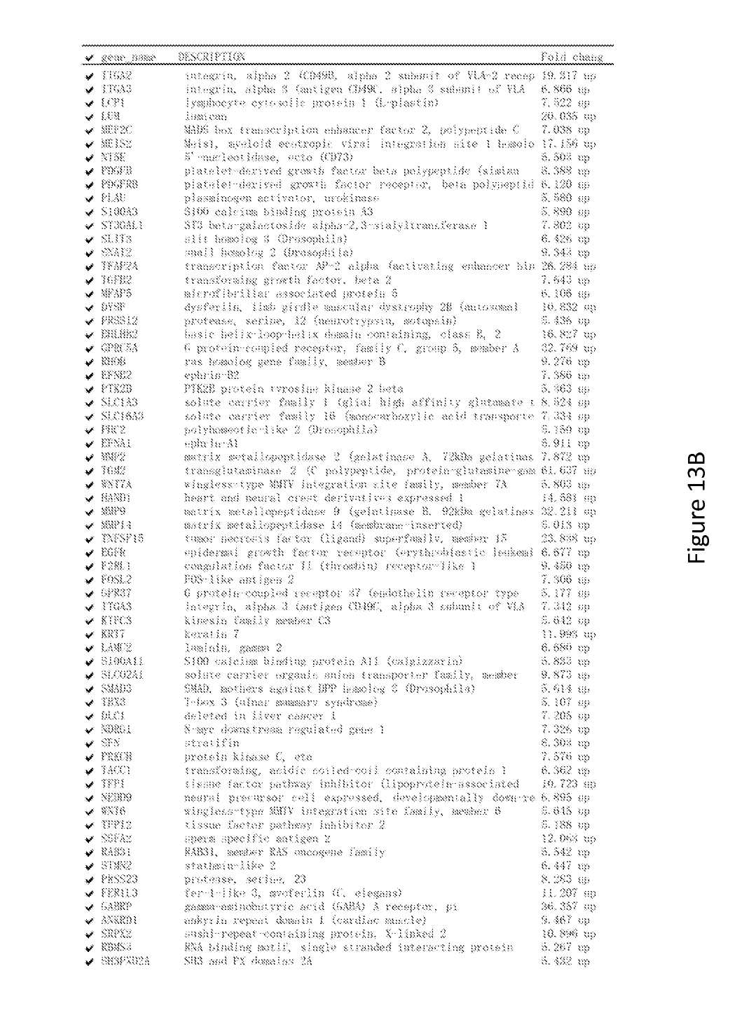

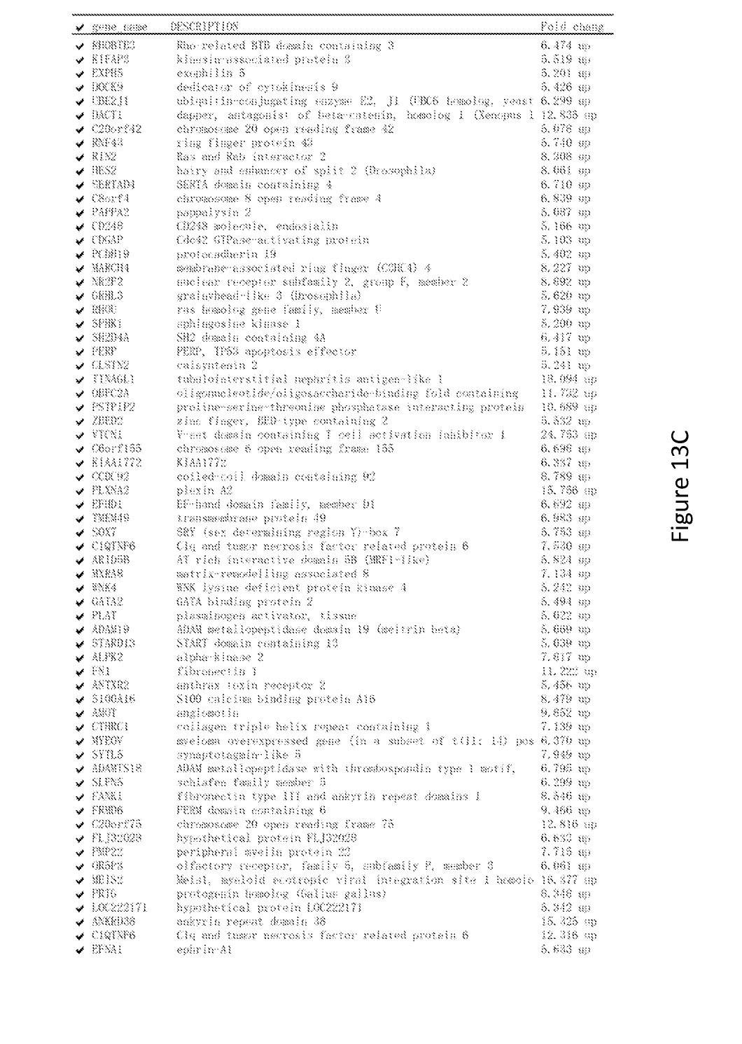

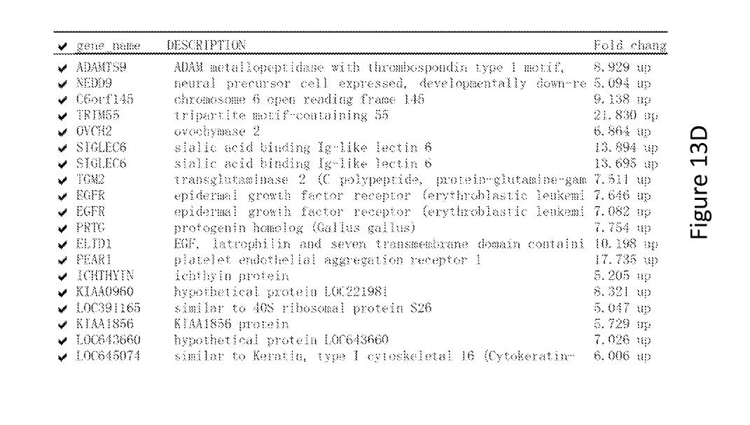

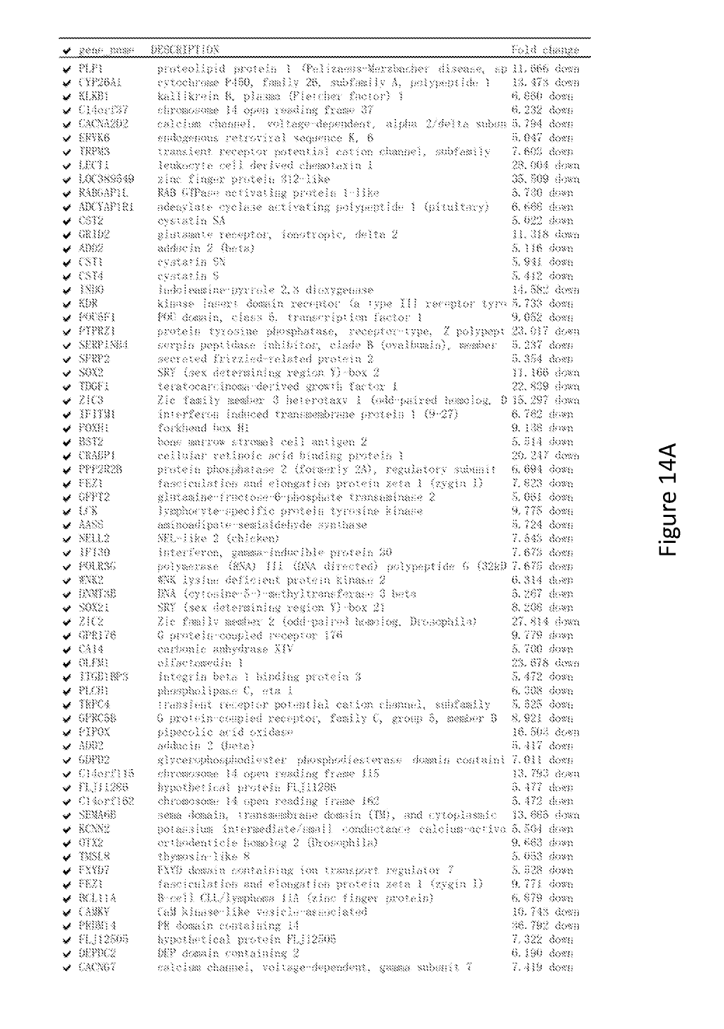

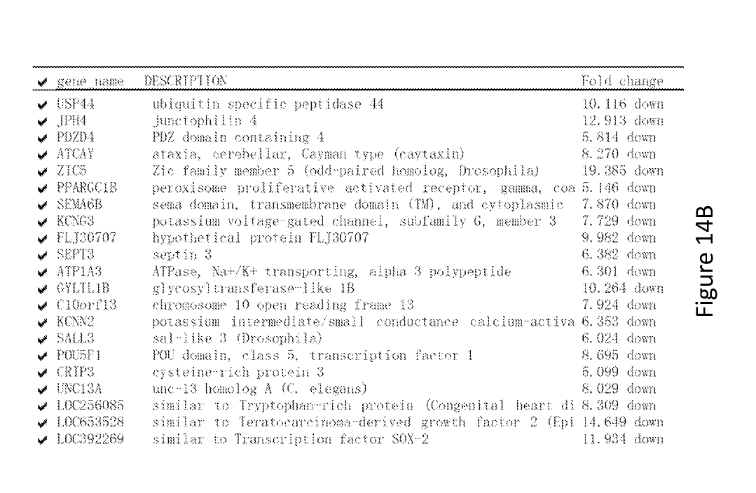

Next, the identity of the cellular determinants whose induction or repression by the hepatic specification process correlated with permissiveness to infection was assessed. Liver-specific genes that are also important for HCV infection are good candidates for such determinants. The microRNA miR-122 is such a cellular cofactor (2002, Lagos-Quintana et al., Curr Biol. 12:735-739; 2005, Jopling et al., Science 309:1577-1581; 2010, Lanford et al., Science 327:198-201). Expression of miR-122 was not detectable by real-time RT-PCR in day 0 or day 4 cells but was greatly induced at day 7 and then maintained throughout the differentiation process (FIG. 5A). These data suggested that the induction of miR-122 expression by hepatic specification conditions contributed to, but was not sufficient for, the transition of non-permissive cells to permissive cells. Next microarray analysis was performed to compare gene expression profiles of day-7 (non-permissive) and day-10 (permissive) cells. The addition of Medium D resulted in changes in expression levels of hundreds of genes, many of which are associated with cell signaling pathways or function of extracellular components (FIGS. 13 and 14)

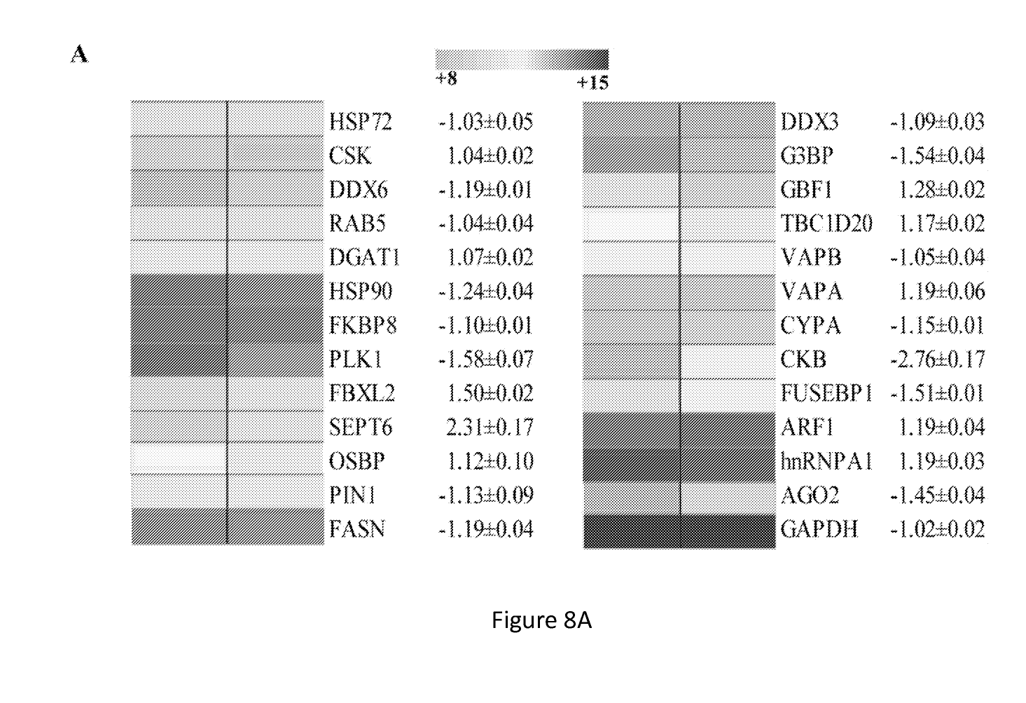

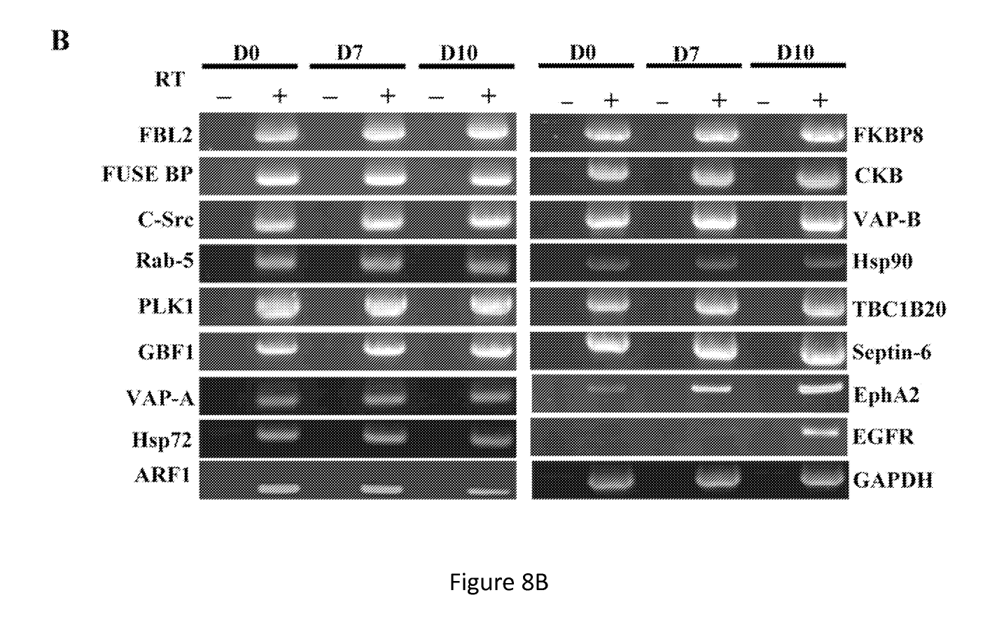

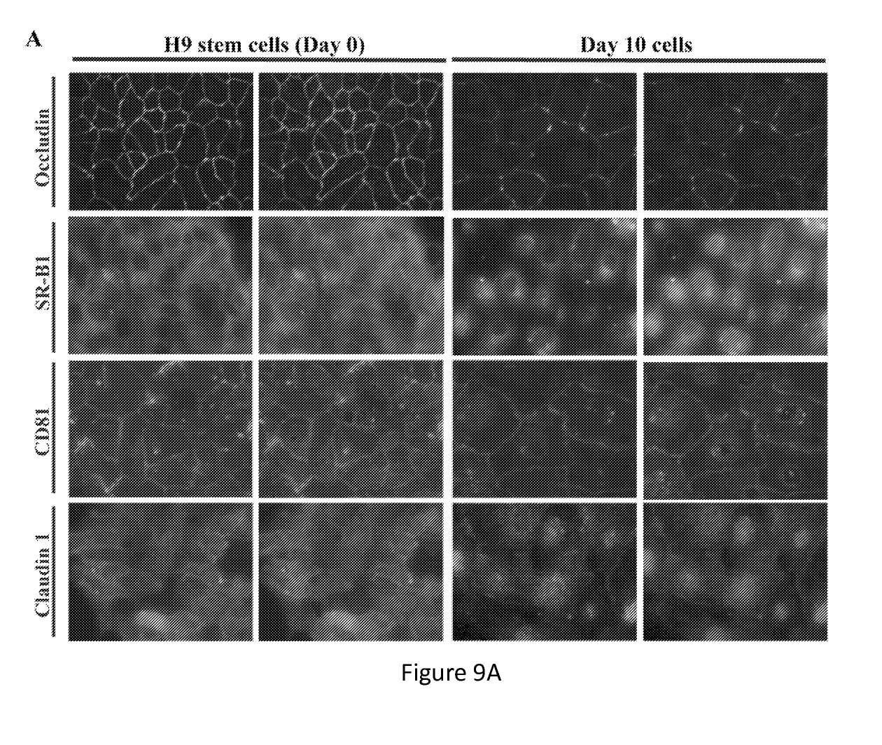

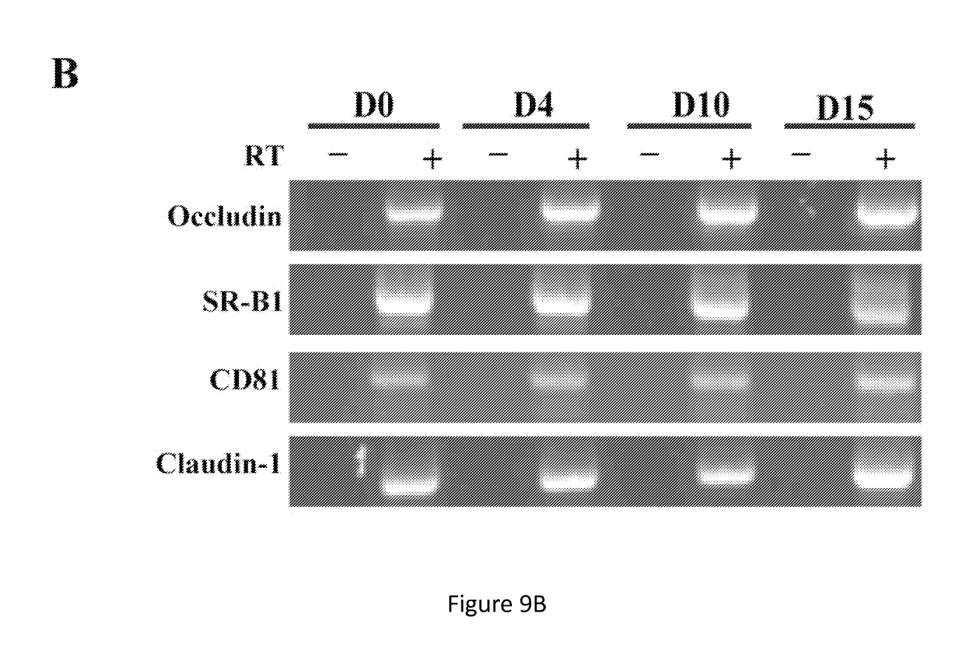

Genes that have been previously implicated in HCV infection were examined. Expression of four well-characterized receptors (i.e., CD81, SR-BI, claudin-1, and occluding) remained largely unchanged. The expression of the putative attachment factor, the low-density lipoprotein receptor (LDL-R), also remained largely unchanged (FIG. 5B). The expression of epidermal growth factor receptor (EGFR) and ephrin receptor A2 (EphA2), two receptor tyrosine kinases (RTKs) identified in a siRNA library screening for HCV entry factors (2011, Lupberger et al., Nat Med. 17:589-595), increased in day-10 (FIG. 5B). Quantitative RT-PCR confirmed the upregulation of these genes (FIG. 5C). In addition, phosphatidylinositol 4-kinase type III alpha (PI4KIIIα), another critical HCV cofactor (2009, Berger et al., Proc Natl Acad Sci USA 106:7577-7582; 2009, Tai et al., Cell Host Microbe. 5:298-307; 2009, Trotard et al., FASEB J. 23:3780-3789), was also induced in day-10 cells, especially at the protein level (FIG. 5D). In contrast, the expression of most other reported cellular cofactors of HCV remained unchanged (FIG. 8). Immunostaining of cell-surface receptors confirmed the RNA data from microarray and conventional RT-PCR (FIG. 9).

Finally, there were also many genes that were down-regulated in day-10 cells compared to day-7 cells. One of these encoded the interferon-induced transmembrane protein 1 (IFITM1), an interferon-stimulated gene (ISG) recently shown to repress HCV replication and down-regulation of which by siRNA enhanced HCVcc infection in Huh-7.5 cells (2011, Raychoudhuri et al., J Virol. 85:12881-12889). Taken together, these results suggest that transition to HCV-permissiveness during the in vitro differentiation process described herein may require the activation of one or more positive factors (miR122, EGFR/EphA2, PI4KIIIα etc.) and downregulation of one or more antiviral genes such as IFITM1.

### Genetic Modification to Generate HCV-Resistant DHHs

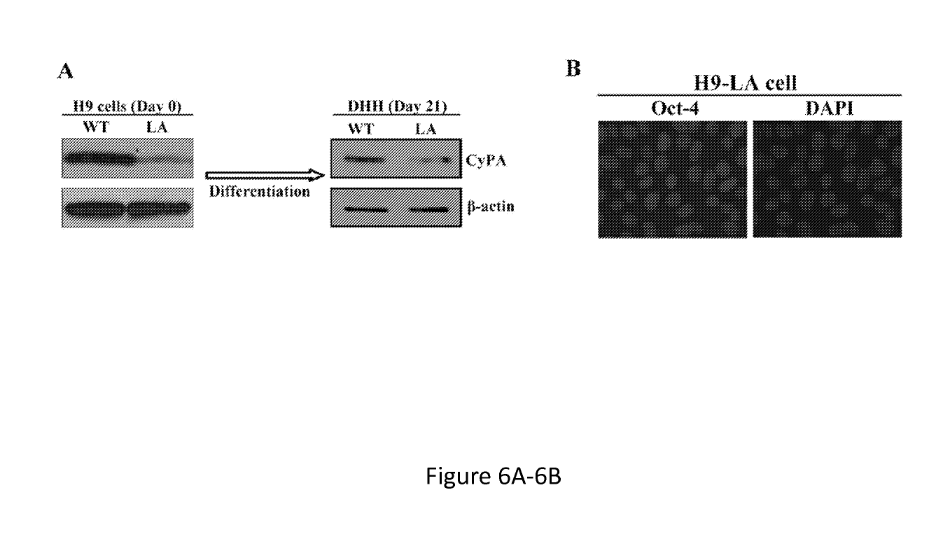

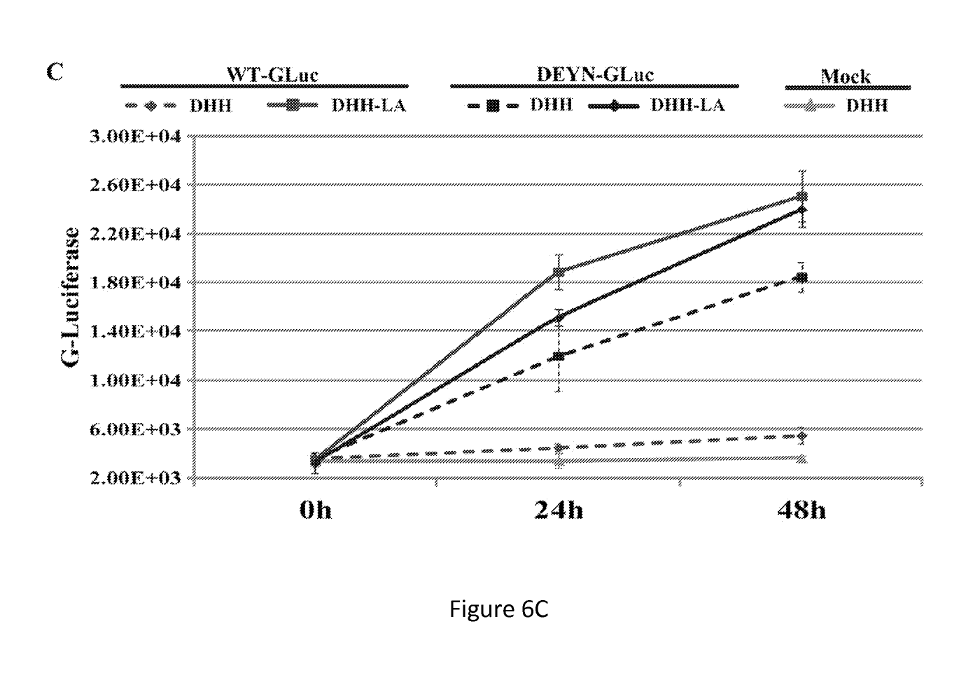

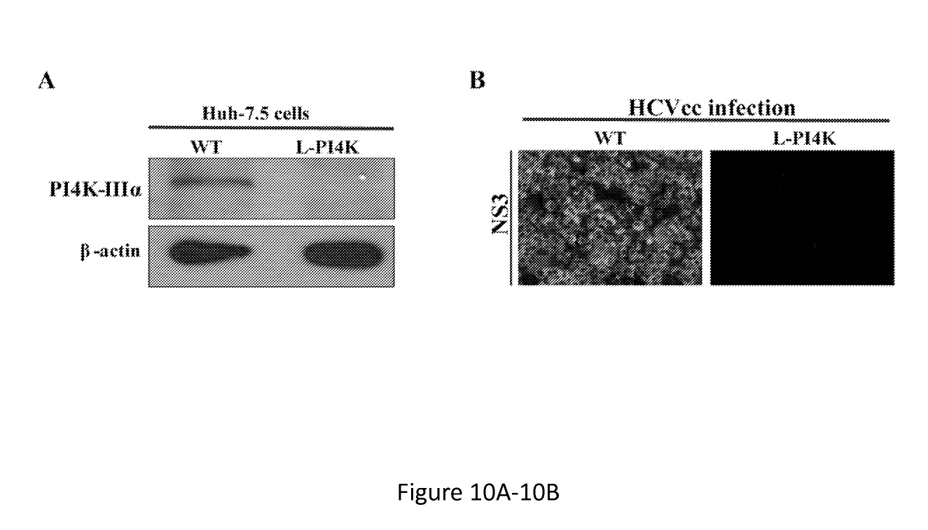

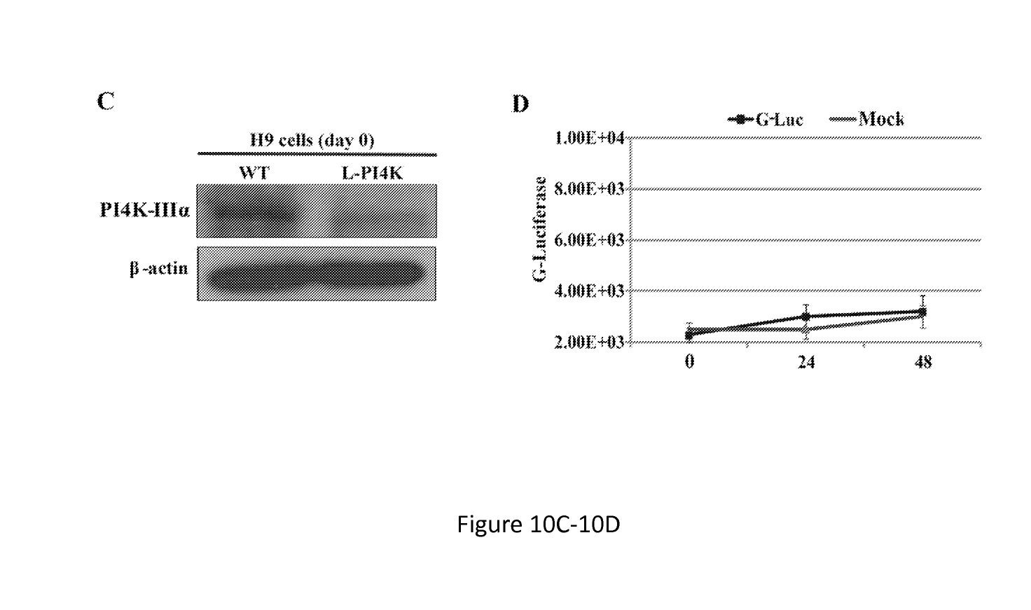

A distinct advantage of DHHs over PHHs is the potential to genetically modify the cells at the pluripotent stage and then produce DHHs with the desired phenotype. A small-hairpin RNA (shRNA) directed at cyclophilin A (CyPA) was introduced into H9 cells by lentiviral vector-mediated gene delivery. This shRNA, sh-A161, had previously been shown to block HCV infection in a human hepatoma cell line Huh-7.5 by knocking down expression of CyPA (2008, Yang et al., J Virol. 82:5269-5278). The importance of CyPA in HCV life cycle has been validated clinically with cyclophilin inhibitors in patient trials (2009, Flisiak et al., Hepatology 49:1460-1468). Suppression of CyPA expression in H9 cells was similarly achieved by stable expression of sh-A161 (FIG. 6A) and the resulting KD cell line (H9-LA) retained normal expression of the pluripotent marker Oct-4 (FIG. 6B). When H9-LA cells were subjected to the same differentiation procedure to produce DHH-LA, the knockdown of CyPA was maintained in the differentiated cells (FIG. 6A), indicating long-term suppression of gene expression by shRNA was not affected by the differentiation step as long as a house-keeping promoter is selected to drive the shRNA expression (e.g. a murine U6 promoter contained in the lentiviral construct used in this study). Infection by wildtype HCVcc, however, was reduced to the mock level in DHH-LA (FIG. 6C, dotted lines) cells. Importantly, these cells remained permissive to infection by a CyPA-independent mutant virus (GLuc-DEYN) (FIG. 6C), recently isolated using a genetic approach termed cofactor-independent mutant (CoFIM) selection (2010, Yang et al., PLoS Pathog. 6, e1001118). These data suggest that the block to HCV infection was due to CyPA knockdown, rather than to nonspecific effect of the shRNA expression (2009, Kenworthy et al., Nucleic Acids Res. 37:6587-6599). A second H9 line harboring a shRNA directed at PI4KIIIα also produced HCV-resistant DHHs upon differentiation (FIG. 10), lending further support to the broad utility of the modification/differentiation technology.

### Patient Serum-Derived HCV Infect DHHs but No Huh-7.5 Cells

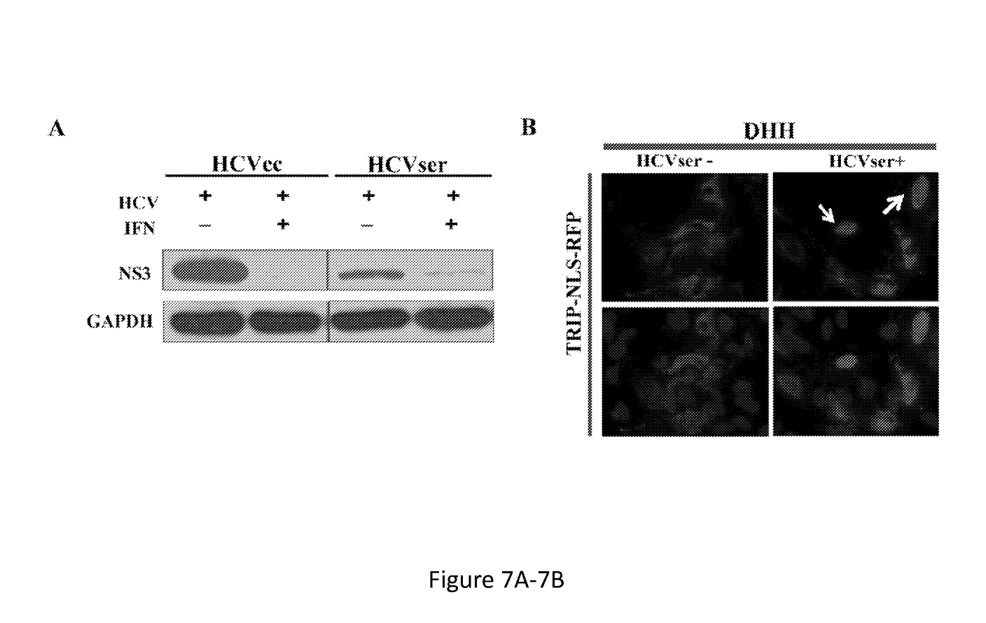

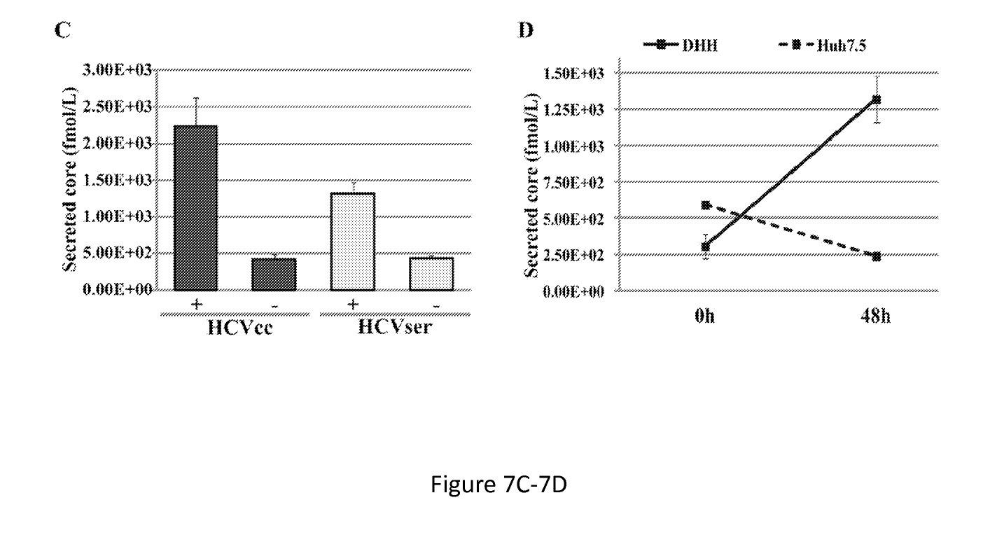

Although robust infection of PHHs by HCVcc has been reported (2010, Podevin et al., Gastroenterology 1355-1364; 2010, Ploss et al., Proc Natl Acad Sci USA 107:3141-3145; 2010, Banaudha et al., Hepatology 51:1922-1932), direct infection by HCV derived from patient serum (HCVser) remained inefficient (2010, Podevin et al., Gastroenterology. 139:1355-1364; 1998, Fournier et al., J Gen Virol. 79:2367-2374). Here, DHHs were infected with a genotype 1b patient serum that contained high-titer HCV RNA copy numbers (1.8×10⁶ copies/ml) for 48 hours before the cells were lysed for analysis of HCV protein expression. Infection was readily detectable by western blotting and sensitive to IFN inhibition (FIG. 7A), although the infection signal of HCVser was weaker than that of the HCVcc. HCVser infection was also detectable with the HDFR assay (FIG. 7B). In addition, secretion of HCV core antigen was detected in the supernatant of the DHHs infected by HCVser (FIG. 7C). In contrast, infection of Huh-7.5 cells with the same multiplicity of infection (m.o.i=0.5) of HCVser did not result in detectable intracellular expression of NS3 or any release of HCV core into the culture media (FIG. 7D). Given the high permissiveness of Huh-7.5 cells to HCVcc infection, these results strongly suggest that HCVser preferentially infects the non-cancerous DHHs.

### Example 2

### Infection of Stem Cell-Derived Human Hepatocytes by Hepatitis B Virus

Infection of the DHHs was performed at day 10, 11, 12, 13, 14, 15, 16, 17, and 18 of the DHH differentiation protocol. Infection was conducted in the presence or in the absence of 4% PEG-6000 and 2% DMSO for 16 hours. In some experiments where infection was performed at day 10, 11, 12, 13, and 14, a second round infection at 48 hours after the first infection is also conducted. The culture supernatants were collected every 2 days and cell lysates were collected at day 21. All the supernatant and lysate samples were subjected to ELISA and western blotting to detect of HBV core antigen.

Two types of HBV particle preparation are used for the DHH infection experiments. First, HBV particles were produced in cell culture with a titer of 3e10/ml. Using the first preparation, the infection was conducted using a multiplicity of infection (M.O.I) of at least 100. Second, HBV particles were obtained from a serum sample of a transgenic mouse that expresses the HBV genome and proteins. The second preparation has a titer of 10e8/ml genomes.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of making a Hepatitis C Virus (HCV)-permissive Differentiated Human Hepatocyte-Like Cell (DHH), the method comprising the steps of:
a. contacting at least one stem cell with a first cell culture medium comprising, activin-A, b-FGF and Wnt-3A and incubating the at least one stem cell for about 24 hours; then
b. contacting at least one cell from step (a) with a second cell culture medium comprising, activin-A and b-FGF and incubating the at least one cell from step (a) for about 3 days; then
c. contacting at least one cell from step (b) with a third cell culture medium comprising FGF-10 and incubating the at least one cell from step (b) for about 3 days; then
d. contacting at least one cell from step (c) with a fourth cell culture medium comprising, FGF-10, retinoic acid, and SB431542 and incubating the at least one cell from step (c) for about 3 days.

2. The method of claim 1, wherein the stem cell is a pluripotent stem cell.

3. The method of claim 2, wherein the pluripotent stem cell is an embryonic stem cell (ESC) or an induced pluripotent stem cell (iPSC).

4. The method of claim 1, wherein at least one of the first, second, third, and fourth cell culture medium further comprises at least one of 0-20% Probumin®, 0-2% β-Mercaptoethanol, 0-5% L-Alanyl-L-glutamine, and 0-5% hESC supplement.

5. The method of claim 1, wherein the first cell culture medium comprises 1-1000 ng/ml activin-A, 0.1-50 ng/ml b-FGF, and 0.1-1000 ng/ml Wnt-3A.

6. The method of claim 1, wherein the second cell culture medium comprises 1-1000 ng/ml activin-A and 0.1-50 ng/ml b-FGF.

7. The method of claim 1, wherein the third cell culture medium comprises 0.1-500 ng/ml FGF-10.

8. The method of claim 1, wherein the fourth cell culture medium comprises 0.1-500 ng/ml FGF-10, 0.01-10 μM retinoic acid, and 0.1-100 μM SB431542.

9. The method of claim 1, wherein the HCV-permissive DHH is permissive for infection by HCVser.

10. The method of claim 1, wherein the HCV-permissive DHH is permissive for infection by at least one of the HCV genotypes selected from the group consisting of genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a and 6a.

11. The method of claim 1, comprising the additional step of contacting at least one cell that was contacted with the first, second, third, and fourth cell culture mediums, with a fifth cell culture medium comprising, FGF-4, EGF, and HGF and incubating the at least one cell from about 1 day to about 10 days.

12. The method of claim 11, wherein the fifth culture medium comprises 0.1-100 ng/ml FGF-4, 0.1-1000 ng/ml EGF, and 0.1-1000 ng/ml HGF.

13. A composition comprising an HCV-permissive DHH.

14. The composition of claim 13, wherein the HCV-permissive DHH is permissive for infection by HCVser.

15. The composition of claim 13, wherein the HCV-permissive DHH is permissive for infection by at least one of the HCV genotypes selected from the group consisting of genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a and 6a.

16. A composition comprising an HCV-permissive DHH made according to the method of claim 1.

17. A HCV culture system comprising an HCV-permissive DHH and an HCV.

18. The HCV culture system of claim 17, wherein the HCV-permissive DHH is permissive for infection by HCVser.

19. The HCV culture system of claim 17, wherein the HCV-permissive DHH is permissive for infection by at least one of the HCV genotypes selected from the group consisting of genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a and 6a.

20. The HCV culture system of claim 17, wherein the HCV is resistant to at least one drug.

21. A method of identifying a test compound as a modulator of HCV infection, the method comprising:
a. placing at least one HCV-permissive DHH in culture medium in a first container,
b. contacting the at least one HCV-permissive DHH in the first container with an HCV in the absence of the test compound,
c. determining the level of HCV in the culture medium in the first container in the absence of the test compound,
d. placing at least one HCV-permissive DHH in culture medium in a second container,
e. contacting the at least one HCV-permissive DHH in the second container with an HCV in the presence of the test compound,
f. determining the level of HCV in the culture medium in the second container in the presence of the test compound,
g. comparing the level of HCV in the presence of the test compound with the level of HCV in the absence of the test compound,
h. identifying the test compound as a modulator of HCV infection when the level of HCV in the presence of the test compound is different than level of HCV in the absence of the test compound.

22. The method of claim 21, wherein when the level of HCV is higher in the presence of the test compound, the test compound is identified as an HCV infection activator.

23. The method of claim 21, wherein when the level of HCV is lower in the presence of the test compound, the test compound is identified as an HCV infection inhibitor.

24. The method of claim 21, wherein the level of HCV is determined by measuring the HCV titer.

25. The method of claim 21, wherein the level of HCV is determined by measuring the level of an HCV nucleic acid.

26. The method of claim 21, wherein the level of HCV is determined by measuring the level of an HCV polypeptide.

27. The method of claim 21, wherein the test compound is at least one selected from the group consisting of: a chemical compound, a protein, a peptide, a peptidomemetic, an antibody, a nucleic acid, an antisense nucleic acid, an shRNA, a ribozyme, and a small molecule chemical compound.

28. The method of claim 21, wherein the HCV-permissive DHH is permissive for infection by HCVser.

29. The method of claim 21, wherein the HCV-permissive DHH is permissive for infection by at least one of the HCV genotypes selected from the group consisting of genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a and 6a.

30. The method of claim 21, wherein the HCV is resistant to at least one drug.

31. A container comprising an HCV-permissive DHH.

32. The container of claim 31, wherein the HCV-permissive DHH is permissive for infection by HCVser.

33. The container of claim 31, wherein the HCV-permissive DHH is permissive for infection by at least one of the HCV genotypes selected from the group consisting of genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a and 6a.

34. A kit comprising an HCV-permissive DHH and instructional material.

35. The kit of claim 34, wherein the HCV-permissive DHH is permissive for infection by HCVser.

36. The kit of claim 34, wherein the HCV-permissive DHH is permissive for infection by at least one of the HCV genotypes selected from the group consisting of genotype 1a, 1b, 2a, 2b, 2c, 2d, 3a, 3b, 3c, 3d, 3e, 3f, 4a, 4b, 4c, 4d, 4e, 4f, 4g, 4h, 4i, 4j, 5a and 6a.

37. A method of making a Hepatitis B Virus (HBV)-permissive Differentiated Human Hepatocyte-Like Cell (DHH), the method comprising the steps of:
a. contacting at least one stem cell with a first cell culture medium comprising, activin-A, b-FGF and Wnt-3A and incubating the at least one cell for about 24 hours; then
b. contacting at least one cell from step (a) with a second cell culture medium comprising, activin-A and b-FGF and incubating the at least one cell from step (a) for about 3 days; then
c. contacting at least one cell from step (b) with a third cell culture medium comprising FGF-10 and incubating the at least one cell from step (b) for about 3 days; then
d. contacting the at least one cell from step (c) with a fourth cell culture medium comprising, FGF-10, retinoic acid, and SB431542 and incubating the at least one cell from step (c) for about 3 days.

38. The method of claim 37, wherein the stem cell is a pluripotent stem cell.

39. The method of claim 38, wherein the pluripotent stem cell is an embryonic stem cell (ESC) or an induced pluripotent stem cell (iPSC).

40. The method of claim 37, wherein at least one of the first, second, third, and fourth cell culture medium further comprises at least one of 0-20% Probumin®, 0-2% β-Mercaptoethanol, 0-5% L-Alanyl-L-glutamine, and 0-5% hESC supplement.

41. The method of claim 37, wherein the first cell culture medium comprises 1-1000 ng/ml activin-A, 0.1-50 ng/ml b-FGF, and 0.1-1000 ng/ml Wnt-3A.

42. The method of claim 37, wherein the second cell culture medium comprises 1-1000 ng/ml activin-A and 0.1-50 ng/ml b-FGF.

43. The method of claim 37, wherein the third cell culture medium comprises 0.1-500 ng/ml FGF-10.

44. The method of claim 37, wherein the fourth cell culture medium comprises 0.1-500 ng/ml FGF-10, 0.01-10 μM retinoic acid, and 0.1-100 μM SB431542.

45. The method of claim 37, wherein the HBV-permissive DHH is permissive for infection by HBVser.

46. The method of claim 37, wherein the HBV-permissive DHH is permissive for infection by at least one of the HBV genotypes selected from the group consisting of genotype A, B, C, D, E, F, G and H.

47. The method of claim 37, comprising the additional step of contacting at least one cell that was contacted with the first, second, third, and fourth cell culture mediums, with a fifth cell culture medium comprising, FGF-4, EGF, and HGF and incubating the at least one cell from about 1 day to about 10 days.

48. The method of claim 47, wherein the fifth culture medium comprises 0.1-100 ng/ml FGF-4, 0.1-1000 ng/ml EGF, and 0.1-1000 ng/ml HGF.

49. A composition comprising an HBV-permissive DHH.

50. The composition of claim 49, wherein the HBV-permissive DHH is permissive for infection by HBVser.

51. The composition of claim 49, wherein the HBV-permissive DHH is permissive for infection by at least one of the HBV genotypes selected from the group consisting of genotype A, B, C, D, E, F, G and H.

52. A composition comprising an HBV-permissive DHH made according to the method of claim 1.

53. A HBV culture system comprising an HBV-permissive DHH and an HBV.

54. The HBV culture system of claim 53, wherein the HBV-permissive DHH is permissive for infection by HBVser.

55. The HBV culture system of claim 53, wherein the HBV-permissive DHH is permissive for infection by at least one of the HBV genotypes selected from the group consisting of genotype A, B, C, D, E, F, G and H.

56. The HBV culture system of claim 53, wherein the HBV is resistant to at least one drug.

57. A method of identifying a test compound as a modulator of HBV infection, the method comprising:
a. placing at least one HBV-permissive DHH in culture medium in a first container,
b. contacting the at least one HBV-permissive DHH in the first container with an HBV in the absence of the test compound,
c. determining the level of HBV in the culture medium in the first container in the absence of the test compound,
d. placing at least one HBV-permissive DHH in culture medium in a second container,
e. contacting the at least one HBV-permissive DHH in the second container with an HBV in the presence of the test compound,
f. determining the level of HBV in the culture medium in the second container in the presence of the test compound,
g. comparing the level of HBV in the presence of the test compound with the level of HBV in the absence of the test compound,
h. identifying the test compound as a modulator of HBV infection when the level of HBV in the presence of the test compound is different than level of HBV in the absence of the test compound.

58. The method of claim 57, wherein when the level of HBV is higher in the presence of the test compound, the test compound is identified as an HBV infection activator.

59. The method of claim 57, wherein when the level of HBV is lower in the presence of the test compound, the test compound is identified as an HBV infection inhibitor.

60. The method of claim 57, wherein the level of HBV is determined by measuring the HBV titer.

61. The method of claim 57, wherein the level of HBV is determined by measuring the level of an HBV nucleic acid.

62. The method of claim 57, wherein the level of HBV is determined by measuring the level of an HBV polypeptide.

63. The method of claim 57, wherein the test compound is at least one selected from the group consisting of: a chemical compound, a protein, a peptide, a peptidomemetic, an antibody, a nucleic acid, an antisense nucleic acid, an shRNA, a ribozyme, and a small molecule chemical compound.

64. The method of claim 57, wherein the HBV-permissive DHH is permissive for infection by HBVser.

65. The method of claim 57, wherein the HBV-permissive DHH is permissive for infection by at least one of the HBV genotypes selected from the group consisting of genotype A, B, C, D, E, F, G and H.

66. The method of claim 57, wherein the HBV is resistant to at least one drug.

67. A container comprising an HBV-permissive DHH.

68. The container of claim 67, wherein the HBV-permissive DHH is permissive for infection by HBVser.

69. The container of claim 67, wherein the HBV-permissive DHH is permissive for infection by at least one of the HBV genotypes selected from the group consisting of genotype A, B, C, D, E, F, G and H.

70. A kit comprising an HBV-permissive DHH and instructional material.

71. The kit of claim 70, wherein the HBV-permissive DHH is permissive for infection by HBVser.

72. The kit of claim 70, wherein the HBV-permissive DHH is permissive for infection by at least one of the HBV genotypes selected from the group consisting of genotype A, B, C, D, E, F, G and H.
